(19) United States
(12) Patent Application Publication
(10) Pub. No.: US 2014/0004105 A1
(43) Pub. Date: Jan. 2, 2014

(54) AGE-RELATED MACULAR DEGENERATION DIAGNOSTICS

(71) Applicant: SEQUENOM, INC., (US)

(72) Inventor: Lorah Terese PERLEE, Wilton, CT (US)

(73) Assignee: SEQUENOM, INC., San Diego, CA (US)

(21) Appl. No.: 13/781,257

(22) Filed: Feb. 28, 2013

Related U.S. Application Data
(60) Provisional application No. 61/666,756, filed on Jun. 29, 2012.
(60) Provisional application No. 61/666,753, filed on Jun. 29, 2012.
(60) Provisional application No. 61/675,269, filed on Jul. 24, 2012.

(51) Int. Cl. C12Q 1/68

(52) U.S. Cl. CPC C12Q 1/6883
USPC 424/133.1; 506/2

(57) ABSTRACT

The technology relates in part to methods for diagnosing and treating age-related macular degeneration.

### RELATED APPLICATIONS

This patent application claims the benefit of U.S. Provisional Patent Application No. 61/675,269 filed on Jul. 24, 2012, entitled AGE-RELATED MACULAR DEGENERATION DIAGNOSTICS, naming Lorah Terese Perlee as inventor, and designated by Attorney Docket No. SEQ-6047-PV2; U.S. Provisional Patent Application No. 61/666,756 filed on Jun. 29, 2012, entitled AGE-RELATED MACULAR DEGENERATION DIAGNOSTICS, naming Lorah Terese Perlee as inventor, and designated by Attorney Docket No. SEQ-6047-PV; and U.S. Provisional Patent Application No. 61/666,753 filed on Jun. 29, 2012, entitled AGE-RELATED MACULAR DEGENERATION DIAGNOSTICS, naming Lorah Terese Perlee as inventor, and designated by Attorney Docket No. SEQ-6046-PV. The entire content of the foregoing applications is incorporated herein by reference, including all text, tables and drawings.

### FIELD

The technology relates in part to methods for diagnosing and treating age-related macular degeneration.

### BACKGROUND

Age-related macular degeneration (AMD) is the leading cause of irreversible blindness in developed countries. AMD is defined as an abnormality of the retinal pigment epithelium (RPE) that leads to overlying photoreceptor degeneration of the macula and consequent loss of central vision. Early AMD is characterized by drusen (greater than 63 um) and hyper-pigmentation or hypo-pigmentation of the RPE. Intermediate AMD is characterized by the accumulation of focal or diffuse drusen (greater than 120 um) and hyper-pigmentation or hypo-pigmentation of the RPE. Advanced AMD is associated with vision loss due to either geographic atrophy of the RPE and photoreceptors (dry AMD) or choroidal neovascularization (CNV), i.e., neovascular choriocapillary invasion across Bruch's membrane into the RPE and photoreceptor layers (wet AMD). AMD often leads to a loss of central visual acuity, and can progress in a manner that results in severe visual impairment and blindness. Visual loss in wet AMD is more sudden and may be more severe than in dry AMD. The clinical presentation and natural course of AMD are highly variable. The disease may present as early as the fifth decade of life or as late as the ninth decade. The clinical symptoms of AMD range from no visual disturbances in early disease to profound loss of central vision in the advanced late stages of the disease.

Besides age, genetic background often is the most significant non-modifiable risk factor for all stages of AMD, while smoking often is the most significant modifiable risk factor. In some instances, certain loci on chromosome 1 and chromosome 10 (e.g., the complement factor H (CFH) and the age-related maculopathy susceptibility protein 2 (ARMS2)/high temperature requirement factor A1 (HTRA1) genes, respectively) are significantly associated with AMD risk and protection in populations of various ethnicities. In some instances, disregulation of the complement cascade may be a critical early predisposing step in the development of AMD. In some instances, CFH variants are associated with AMD risk. For example, associations are observed between AMD and risk/protective variants in various complement pathway-associated genes, including complement component 2 (C2), complement factor B (CFB), complement component 3 (C3), complement factor H-related 1 and 3 (CFHR1 and CFHR3) and complement factor I (CFI).

### SUMMARY

Provided herein, in some aspects, are methods for predicting a therapeutic effect for treating a disorder, comprising (a) determining a genotype at multiple polymorphic markers for nucleic acid from a subject; (b) predicting a therapeutic effect for treating the disorder based on a composite of the markers, which composite factors in (i) the genotype at each of the markers, and (ii) a coefficient associated with predicting the therapeutic effect for treating the disorder for each of the markers.

Also provided, in some aspects, are methods for predicting a phenotypic subtype of a disorder, comprising (a) determining a genotype at multiple polymorphic markers for nucleic acid from a subject; (b) predicting a phenotypic subtype of the disorder based on a composite of the markers, which composite factors in (i) the genotype at each of the markers, and (ii) a coefficient associated with predicting the phenotypic subtype of the disorder for each of the markers.

Also provided, in some aspects, are methods for determining risk of developing a disorder, comprising (a) determining the genotype at multiple polymorphic markers for nucleic acid from a subject; and (b) determining the risk of developing the disorder based on a composite of the markers, which composite factors in the genotype at each of the sites and a coefficient associated with the risk of developing the disorder for each of the sites.

In some embodiments, the composite also factors an associated risk value for each marker. In some embodiments, the associated risk value is an adjusted log-odds ratio.

In some embodiments, a method comprises multiplying the coefficient by the associated risk value, thereby generating a product for each marker. In some embodiments, a method comprises generating a sum of the products.

In some embodiments, predicting a therapeutic effect for treating a disorder and/or predicting a phenotypic subtype of a disorder comprises determining a risk score that factors in the adjusted log-odds ratio for each marker. In some embodiments, predicting a therapeutic effect for treating a disorder and/or predicting a phenotypic subtype of a disorder comprises determining a risk score that factors in an individual's genotype, adjusted log-odds ratio and residual risk value. In some embodiments, the risk score Sj is calculated according to Equation A:

Sj=intercept+Σ(i to n)βi*Xi Equation A

where Sj is the risk score for subject j, βi is the adjusted log-odds ratio for Xi, the additively coded genotype at marker i, and n is the total number of markers. In some embodiments, predicting a therapeutic effect for treating a disorder and/or predicting a phenotypic subtype of a disorder comprises determining a mean risk score. In some embodiments, predicting a therapeutic effect for treating a disorder and/or predicting a phenotypic subtype of a disorder comprises determining the probability pj according to Equation B:

pj=exp(Sj)/[1+exp(Sj)] Equation B.

In some embodiments, risk score or probability is adjusted by one or more non-genetic factors. The one or more non-genetic factors sometimes comprise one or more of BMI, education status and smoking. In some embodiments, risk score or probability is not adjusted by one or more non-genetic factors.

In some embodiments, one or more of the markers are single nucleotide polymorphic markers. In some embodiments, one or more of the single nucleotide polymorphic markers are in one or more genes chosen from age-related maculopathy susceptibility protein 2 (ARMS2), complement factor H (CFH), complement component 2 (C2), complement component 3 (C3), coagulation factor XIII B subunit (F13B), complement factor H-related 4 (CFHR4), complement factor H-related 5 (CFHR5), and complement factor B (CFB). In some embodiments, one or more of the single nucleotide polymorphic markers are in one or more genes chosen from age-related maculopathy susceptibility protein 2 (ARMS2), complement factor H (CFH), and complement factor H-related 5 (CFHR5). In some embodiments, one or more of the single nucleotide polymorphic markers are in one or more genes chosen from age-related maculopathy susceptibility protein 2 (ARMS2) and complement factor B (CFB).

In some embodiments, one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924, rs2230199, rs11200638, rs1061147, rs1329422, rs2300430, rs10801553, rs1329421, rs10801554, rs7529589, rs1329424, rs572515, rs10922152, rs203674, rs393955, rs381974, rs395544, rs3800390, rs3748557, rs12755054, rs1759016, and rs4151667. In some embodiments, one, two, three, four, five, six, seven, eight, nine, ten, eleven, twelve, thirteen or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

In some embodiments, the markers are rs1061170, rs2274700, rs403846, rs12144939, rs1409153, rs1750311, rs10922153, rs698859, rs2990510, rs9332739, rs641153, rs10490924 and rs2230199. In some embodiments, the markers comprise rs1061170, rs403846, rs1750311, rs10922153, rs10490924. In some embodiments, the markers comprise rs10490924 and rs641153.

In some embodiments, the disorder is late stage acute macular degeneration (AMD). In some embodiments, the late stage AMD is choroidal neovascular (CNV) disease. In some embodiments, the therapeutic giving rise to the therapeutic effect comprises an anti-vascular endothelial growth factor (anti-VEGF) therapeutic. In some embodiments, the therapeutic comprises Ranibizumab. In some embodiments, the phenotypic subtype is bilateral CNV. In some embodiments, the phenotypic subtype is retinal pigment epithelial detachment (RPED) CNV.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a calculation of risk score for two case study patients using 13 risk variants (SNPs) within eight genes associated with AMD.

FIG. 2 shows probability of risk versus risk score.

FIG. 3 shows a ROC (receiver operating characteristic) curve for validation. The sensitivity and specificity of predictions were calculated for an independent dataset using the test panels presented in FIG. 9.

FIG. 4 shows probability of choroidal neovascular (CNV) disease, calculated for the validation dataset (presented in FIG. 6). Shaded bars (i.e., left bar for each pair; marked with a “*”) represent controls and black bars represent patients with CNV disease.

FIG. 5 presents a table showing number of cases (CNV disease) and controls in individual cohorts.

FIG. 6 presents a table showing single nucleotide polymorphisms employed in first stage AMD.

FIG. 7 presents a table showing homogeneity of variance.

FIG. 8 presents a table showing univariate association between demographic factors, genetic factors and risk of choroidal neovascular (CNV) disease.

FIG. 9 presents a table showing a calculation of choroidal neovascular disease risk score: S=intercept (i.e., residual risk)+Σ(i=1 to 13) βi*Xi, where β (regression coefficient; associated risk value) and X (genotype coefficient) are as presented in the table.

FIG. 10 presents a table showing area under the curve for training, tenfold cross-validation and independent validation on a 13-SNP model. SNP, single nucleotide polymorphism; CNV, choroidal neovascular; ROC, receiver operating characteristic

FIG. 11 presents a table showing a comparison of a 13-SNP model with and without demographic factors. There was no significant difference between the two models.

FIG. 12 shows a classification table.

FIG. 13 presents a table showing a comparison of models containing different numbers of single nucleotide polymorphisms (SNPs).

FIG. 14 shows logistic regression results.

### DETAILED DESCRIPTION

Provided herein are methods for predicting therapeutic effects for treating age-related macular degeneration (AMD); methods for predicting phenotypic subtypes related to age-related macular degeneration and methods for estimating the risk of developing late-stage neovascular age-related macular degeneration (AMD). Similar to predictive tests for estimating the risk of developing (AMD), predicting therapeutic effects and phenotypic subtypes can be subject to unique challenges. For example, AMD prevalence increases with age, clinical phenotypes are heterogeneous and control collections are prone to high false-negative rates, as many control subjects are likely to develop disease with advancing age. Risk prediction tests, for example, typically include use of genetic markers in combination with a range of self-reported non-genetic variables such as, for example, body mass index (BMI) and smoking history. Provided herein are predictive methods based largely on genetic markers, which are static through life and not subject to misreporting. Such methods can include an assessment of a panel of single nucleotide polymorphisms (SNPs) as a test for predicting a therapeutic effect for treating certain types of AMD and/or predicting phenotypic subtypes of AMD. In some embodiments, a predictive model based solely on genetic markers is used. In some embodiments, self-reported variables (e.g., smoking history) or non-static factors (BMI, education status) are included. In some embodiments, self-reported variables (e.g., smoking history) or non-static factors (BMI, education status) are not included.

### Predicting a Therapeutic Effect

In some embodiments, methods for predicting a therapeutic effect for treating a disorder are provided. In some embodiments, methods for predicting a phenotypic subtype for a disorder are provided, which are described in further detail below. Predicting a therapeutic effect for treating a disorder refers to predicting a likely outcome of a particular therapeutic with respect to disorder progression and/or symptoms. A therapeutic effect may include, for example, (i) preventing a disorder from occurring (e.g. prophylaxis); (ii) inhibiting the disorder or arresting its development; and/or (iii) relieving, ameliorating, alleviating, lessening, diminishing and/or removing symptoms of a disorder. Predicting a therapeutic effect may occur prior to the initiation of a treatment regimen and/or during an existing treatment regimen. Predicting a therapeutic effect may allow a physician or medical care provider to assign an appropriate treatment regimen to a patient prior to or at the onset of a disorder and/or alter an existing treatment regimen.

In some embodiments, an analysis for predicting a therapeutic effect for a therapy is conducted for a subject, and in instances where there is a prediction of a therapeutic effect for the subject, the therapy is administered to the subject. A prediction sometimes comprises a call or score. A prediction can be determined and/or provided with a particular measure of certainty (e.g., confidence and the like), as described herein. A therapy sometimes comprises administering a therapeutic agent to a subject, and sometimes the therapeutic agent is useful for treating AMD, a late stage of AMD, or a particular type of AMD or late stage AMD (e.g., choroidal neovascular (CNV) disease). AMD and CNV disease therapies are known in the art and non-limiting examples are provided herein. In some instances, an analysis for predicting a therapeutic effect for a therapy is conducted for a subject, and a therapeutic effect for the subject is not predicted (i.e., the therapy is predicted to not elicit a therapeutic effect for the subject). In the latter instances, the therapy often is not administered to the subject.

In some embodiments, predicting a therapeutic effect and/or predicting a phenotypic subtype is based on a composite of genetic markers. A composite typically factors in the genotype at each of one or more genetic marker sites and a coefficient for each genotype (sometimes referred to as genotype coefficient) associated with predicting the therapeutic effect and/or predicting a phenotypic subtype. For a given polymorphic marker, having alleles G and T, for example, one of the alleles is associated with therapeutic effect A and/or phenotypic subtype X, while the other allele is either neutral or protective or associated with a different therapeutic effect and/or phenotypic subtype compared to the average population. Thus, an individual's composite for predicting a therapeutic effect and/or predicting a phenotypic subtype depends on whether they inherited 0, 1 or 2 copies of a particular allele.

A composite sometimes factors in an associated risk value for each genetic marker. An associate risk value sometimes refers to an adjusted log-odds ratio (OR) or regression coefficient. An adjusted log-odds ratio is a value assigned to a polymorphic marker that reflects its weight as a predictor of given outcome (e.g., therapeutic effect, phenotypic subtype). Adjusted log-odds ratios may be calculated for a given polymorphic marker using a logistic regression method such as the method described in Example 1. Adjusted log-odds ratio values may be positive or negative, depending on the strength of association with a particular outcome. In some embodiments, methods for predicting a therapeutic effect and/or predicting a phenotypic subtype include determining a risk score. In some embodiments, risk score is a representation of an individual's genetic burden associated with a predicted outcome. In some embodiments, risk score factors in an individual's genotype and/or composite of genetic markers. In some embodiments, risk score factors in one or more adjusted log-odds ratios (ORs). In some embodiments, risk score factors in a residual risk value, sometimes referred to as an intercept, which is a component associated with an outcome that is independent of the polymorphic markers. In some embodiments, risk score factors in an individual's genotype (e.g., genotype coefficient), adjusted log-odds ratio and residual risk value for one or more genetic markers. Risk score (Sj) may be calculated using the following formula, for example:

Sj=intercept+Σ(i to n)βi*Xi Equation A

where Sj is the risk score for subject j, βi is the adjusted log-odds ratio for Xi, the additively coded genotype at marker i, and n is the total number of markers. The term “Σ (i to n)” in the above equation refers to the summation of values (e.g., βi*Xi) for markers i to n. For example, in a calculation of risk score using thirteen markers i=1 and n=13, and a summation of values is calculated for all thirteen markers (see e.g., Hageman et al. (2011) Human Genomics 5:420-440, which is incorporated by reference in its entirety).

In some embodiments, a mean risk score is determined for a group of individuals. A mean risk score can sometimes be used to generate a threshold or cutoff value for predicting a particular outcome. In some instances, a mean risk score is used to identify particular phenotypic subtypes and/or therapeutic treatment categories that can be predicted using a method provided herein.

In some embodiments, predicting a therapeutic effect for treating a disorder and/or predicting a phenotypic subtype comprises determining the probability pj according to Equation B:

pj=exp(Sj)/[1+exp(Sj)] Equation B

Methods for calculating risk score and probability, and using risk score for predicting a therapeutic effect and/or predicting a phenotypic subtype are presented in Example 1 and Example 2.

In some embodiments, the disorder is macular degeneration (e.g., age-related macular degeneration (AMD), (ARMD)). AMD also may be referred to as acute macular degeneration, and includes early, middle or late stage AMD. AMD is characterized by damage to the retina, resulting in a loss of vision in the center of the visual field (i.e., macula), and is a major cause of blindness and visual impairment in older adults (e.g., older than 50 years). AMD may occur in “dry” and “wet” forms. In the dry (nonexudative) form, cellular debris often referred to as drusen accumulates between the retina and the choroid, and the retina can become detached. In the wet (exudative) form, which often is more severe, blood vessels grow up from the choroid behind the retina, a process referred to as choroidal neovascularization (CNV), and the retina also can become detached. Wet AMD sometimes is referred to as choroidal neovascular (CNV) age-related macular degeneration (AMD) or CNV. CNV can occur rapidly in individuals with defects in Bruch's membrane, the innermost layer of the choroid. CNV typically is associated with excessive amounts of vascular endothelial growth factor (VEGF). In some embodiments, the disorder is CNV. A therapeutic effect can refer to reducing or stopping the growth of blood vessels, such as for patients with CNV.

CNV may be treated with laser coagulation and/or with medication that can stop and/or reverse the growth of blood vessels. Thus, in some instances, a therapeutic effect can refer to reducing or stopping the growth of blood vessels (e.g., for patients with CNV). For example, CNV may be treated with a therapeutic that comprises an anti-vascular endothelial growth factor (anti-VEGF) medication. Non-limiting examples of anti-VEGF medications include antibody derivatives such as ranibizumab (LUCENTIS); monoclonal antibodies such as bevacizumab (AVASTIN); small molecules that inhibit the tyrosine kinases stimulated by VEGF such as lapatinib (TYKERB), sunitinib (SUTENT), sorafenib (NEXAVAR), axitinib, and pazopanib; and VEGF inhibitors such as THC, Cannabidiol and thiazolidinediones.

In some embodiments, predicting a therapeutic effect includes assigning a patient to a treatment category. Treatment categories may include responsive, sensitive, dependent and/or non-responsive groups. Treatment categories also may include partially responsive, partially sensitive, partially dependent and/or partially non-responsive groups. For example, CNV patients treated with VEGF may be assigned to an anti-VEGF sensitive group, an anti-VEGF dependent group, or an anti-VEGF non-responsive group. An anti-VEGF sensitive group may comprise patients that substantially respond to anti-VEGF therapeutics, with continued effects after withdrawal of the medication. An anti-VEGF dependent group may comprise patients that substantially respond to anti-VEGF therapeutics, however do not experience continued effects after withdrawal of the medication (i.e., are dependent on continued administration of the medication). An anti-VEGF non-responsive group may include patients that do not have a substantial response to anti-VEGF therapeutics.

### Predicting a Phenotypic Subtype

In some embodiments, methods for predicting a phenotypic subtype of a disorder are provided. Methods for predicting a phenotypic subtype may include one or more components of a method for predicting a therapeutic effect, as described above. In some instances, predicting a phenotypic subtype may allow a physician or healthcare provider to predict a therapeutic effect for treating a disorder and/or tailor a treatment regimen to particular symptoms based on the phenotypic subtype prediction. For example, a patient may require higher or lower medication dosing, more frequent or less frequent medication administration, and/or combination therapy. In some cases, certain phenotypic subtypes may be more responsive to therapeutic treatment. In some cases, certain phenotypic subtypes may be less responsive to therapeutic treatment. Phenotypic subtypes typically refer to sub-categories of a disorder characterized by one or more particular symptoms and/or other physical, measurable, observed or perceived manifestations of a disorder. Phenotypic subtypes can be disorder-specific and may vary among individuals afflicted with the same disorder. For example, patients with CNV may be assigned to one or more phenotypic subtypes such as, for example, bilateral, unilateral, classic, RPED (retinal pigment epithelial detachment), polyps, PPP (Peripapillary neovascularization), arteriolarization, and occult (characterized by a slower leak compared to classic CNV). For example, a method provided herein may be used to predict whether a patient will develop bilateral or unilateral forms of CNV.

Methods for predicting a therapeutic effect for treating a disorder and/or predicting a phenotypic subtype of a disorder that involve determining a composite for a set of genetic markers, such as the methods described herein, may be applied to diseases, conditions and/or disorders other than AMD. For example, methods described herein may be applied to asthma, MPGN II, various forms of arthritis such as rheumatoid arthritis, lupus erythematosus, autoimmune heart disease, Celiac disease, diabetes mellitus type 1 and type 2, Sjögren's syndrome, inflammatory bowel disease, ischemia-reperfusion injuries, multiple sclerosis, neurodegenerative conditions such as Alzheimer's disease, glomerulonephritis, Barraquer-Simons Syndrome, ovarian hyperstimulation syndrome, kidney disease, cardiovascular disease, myocardial infarction, and various types of cancer.

### Providing a Prediction

A prediction (e.g., for developing a medical disorder; for a therapeutic effect; for a phenotypic subtype) can be provided with a particular measure of certainty (e.g., confidence and the like). In some embodiments, a prediction is provided with an associated level of accuracy, precision and or confidence. A level of accuracy, precision and/or confidence sometimes is a call rate (e.g., about 90% to about 100% correct call rate), a coefficient of variance (CV), an uncertainty value, a confidence level (e.g., a confidence level of about 95% to about 99%)), the like or combination thereof.

A prediction sometimes is expressed as a risk or probability (e.g., of developing a medical disorder; of a therapeutic effect for treating a disorder; and/or of a phenotypic subtype of a disorder). A prediction sometimes comprises one or more numerical values generated using a method described herein in the context of one or more considerations of probability. A consideration of risk or probability can include, but is not limited to: an uncertainty value, a measure of variability, confidence level, sensitivity, specificity, standard deviation, coefficient of variation (CV) and/or confidence level, Z-scores, Chi values, Phi values, the like or combinations thereof. A consideration of probability can facilitate determining whether a subject is at risk of having, or has, a medical disorder and/or subtype of a disorder; and/or is likely to respond to a particular therapeutic treatment for a disorder, for example.

A prediction sometimes includes a null result. A null result sometimes is a data point between two clusters, or sometimes is a numerical value with a standard deviation that encompasses values for both the presence and absence of an outcome. In some embodiments, a determination indicative of a null result still is useful, and the null result can indicate the need for additional information, a repeat of data generation and/or analysis for rendering a determination.

A prediction can be expressed in any suitable form, and sometimes is expressed as a probability (e.g., odds ratio, p-value), likelihood, value in or out of a cluster, value over or under a threshold value, value within a range (e.g., a threshold range), value with a measure of variance or confidence, or risk factor, associated with the presence or absence of a genetic variation for a subject or sample. In certain embodiments, comparison between samples allows confirmation of sample identity (e.g., allows identification of repeated samples and/or samples that have been mixed up (e.g., mislabeled, combined, and the like)).

In some embodiments, a prediction comprises a value above or below a predetermined threshold or cutoff value (e.g., greater than 1, less than 1), and an uncertainty or confidence level associated with the value. A prediction also can describe an assumption used in data processing. In certain embodiments, a prediction comprises a value that falls within or outside a predetermined range of values (e.g., a threshold range) and the associated uncertainty or confidence level for that value being inside or outside the range. In some embodiments, a prediction comprises a value that is equal to a predetermined value (e.g., equal to 1, equal to zero), or is equal to a value within a predetermined value range, and its associated uncertainty or confidence level for that value being equal or within or outside a range. A prediction sometimes is graphically represented as a plot (e.g., profile plot).

Different methods for generating a prediction sometimes can produce different types of results. A prediction can lead to four types of scores or calls: true positive, false positive, true negative and false negative. Thus, a prediction can be characterized as a true positive, true negative, false positive or false negative in some embodiments. The term “true positive” as used herein refers to a correctly rendered positive prediction for a subject. The term “false positive” as used herein refers to an incorrectly rendered positive prediction for a subject. The term “true negative” as used herein refers to a correctly rendered negative prediction for a subject. The term “false negative” as used herein refers to an incorrectly rendered negative prediction for a subject. Two measures of performance for any given method can be calculated based on ratios of these occurrences: (i) a sensitivity value, which generally is the fraction of predicted positives that are correctly identified as being positives; and (ii) a specificity value, which generally is the fraction of predicted negatives correctly identified as being negative.

The term “sensitivity” as used herein refers to the number of true positives divided by the number of true positives plus the number of false negatives, where sensitivity (sens) may be within the range of 0≦sens≦1. Ideally, the number of false negatives equal zero or close to zero, such that an incorrect negative prediction is not provided or minimized. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where specificity (spec) may be within the range of 0≦spec≦1. Ideally, the number of false positives equal zero or close to zero, such that an incorrect positive prediction is not provided or is minimized.

In certain embodiments, one or more of sensitivity, specificity and/or confidence level are expressed as a percentage. In some embodiments, the percentage, independently for each variable, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome is not due to chance) in certain embodiments is expressed as a Z-score, a p-value, or the results of a t-test. In some embodiments, a measured variance, confidence interval, sensitivity, specificity and the like (e.g., referred to collectively as confidence parameters) is generated for a prediction.

A method (e.g., a method using a particular set of markers) that has sensitivity and specificity equaling one, or 100%, or near one (e.g., between about 90% to about 99%) sometimes is selected for rendering a prediction. In some embodiments, a method having a sensitivity equaling 1, or 100% is selected, and in certain embodiments, a method having a sensitivity near 1 is selected (e.g., a sensitivity of about 90%, a sensitivity of about 91%, a sensitivity of about 92%, a sensitivity of about 93%, a sensitivity of about 94%, a sensitivity of about 95%, a sensitivity of about 96%, a sensitivity of about 97%, a sensitivity of about 98%, or a sensitivity of about 99%). In some embodiments, a method having a specificity equaling 1, or 100% is selected, and in certain embodiments, a method having a specificity near 1 is selected (e.g., a specificity of about 90%, a specificity of about 91%, a specificity of about 92%, a specificity of about 93%, a specificity of about 94%, a specificity of about 95%, a specificity of about 96%, a specificity of about 97%, a specificity of about 98%, or a specificity of about 99%).

A process described herein for rendering a prediction can be transformative. For example, an individual's genotype at one or more markers can be transformed by a method provided herein into a representation of the likelihood of developing a disorder and/or subtype of a disorder, and/or responding to a particular therapeutic treatment. Such a transformed representation often is specifically utilized as part of making a prediction described herein.

### Genetic Markers

In some embodiments, predicting a therapeutic effect and/or predicting a phenotypic subtype includes assessment of one or more genetic markers, as described above. A genetic marker is a nucleic acid sequence or gene, often having a known location on a chromosome, which can be assessed by genotyping nucleic acid from an individual. A genetic marker may comprise a relatively short nucleic acid sequence, such as a sequence surrounding a single base-pair change (single nucleotide polymorphism (SNP)) or microsatellite, or a relatively long nucleic acid sequence, such as a minisatellite.

Genetic markers herein typically include one or more polymorphisms, and thus sometimes are referred to as polymorphic markers. A polymorphism refers to the occurrence of two or more genetically determined alternative sequences or alleles in a population. Each divergent sequence is termed an allele, and can be part of a gene or located within an intergenic or non-gene sequence. Diploid organisms can contain two alleles and may be homozygous or heterozygous for allelic forms. The first identified allelic form often is arbitrarily designated the reference form or allele; other allelic forms are designated as alternative or variant alleles. The most frequently occurring allelic form in a selected population typically is referred to as the wild-type form.

A polymorphic site refers to the position or locus at which sequence divergence occurs at the nucleic acid level and is sometimes reflected at the amino acid level. The polymorphic region or polymorphic site refers to a region of the nucleic acid where the nucleotide difference that distinguishes the variants occurs, or, for amino acid sequences, a region of the amino acid sequence where the amino acid difference that distinguishes the protein variants occurs. A polymorphic site can be as small as one base pair, often termed a “single nucleotide polymorphism” (SNP). The SNPs can be any SNPs in or proximal to loci identified herein, including intragenic SNPs in exons, introns, or upstream or downstream regions of a gene, as well as SNPs that are located outside of gene sequences. Examples of such SNPs include, but are not limited to, those provided herein.

In some embodiments, one or more genotypes are assessed for one or more genetic markers. Genotype refers to one or more polymorphisms of interest found in an individual, for example, within a gene of interest. Diploid individuals have a genotype that comprises two different sequences (heterozygous) or one sequence (homozygous) at a polymorphic site. Methods for assessing genotypes are known in the art, some of which are described herein.

Genetic markers sometimes can be part of a cluster of markers located at adjacent or nearby loci (e.g., haplotype block or haplogroup). Haplotype refers to a nucleotide sequence comprising one or more polymorphisms of interest contained on a subregion of a single chromosome of an individual. A haplotype can refer to a set of polymorphisms in a single gene, an intergenic sequence, or in larger sequences including both gene and intergenic sequences, e.g., a collection of genes, or of genes and intergenic sequences. For example, a haplotype can refer to a set of polymorphisms on a given chromosome within certain genes and/or within intergenic sequences (i.e., intervening intergenic sequences, upstream sequences, and downstream sequences that are in linkage disequilibrium with polymorphisms in the genic region). Haplotype sometimes refers to a set of single nucleotide polymorphisms (SNPs) found to be statistically associated on a single chromosome. A haplotype also can refer to a combination of polymorphisms (e.g., SNPs) and other genetic markers (e.g., a deletion) found to be statistically associated on a single chromosome. A haplotype can be a set of maternally inherited alleles, or a set of paternally inherited alleles, at any locus.

In some embodiments, one or more genetic markers are assessed. In some embodiments, a genetic marker panel is assessed. A genetic marker panel may comprise two or more SNPs. For example, a genetic marker panel can comprise 2, 3, 4, 5, 6, 7, 8, 9, 10, 11, 12, 13, 14, 15, 16, 17, 18, 19, 20 or more SNPs. In some embodiments, a genetic marker panel may comprise 2 SNPs. In some embodiments, a genetic marker panel may comprise 5 SNPs. In some embodiments, a genetic marker panel may comprise 13 SNPs. In some embodiments, SNPs comprise one or more variants found in regulators of a complement activation (RCA) locus spanning, for example, complement factor H (CFH), complement factor H-related 4 (CFHR4), complement factor H-related 5 (CFHR5) and coagulation factor XIII B subunit (F13B) genes. In some embodiments, SNPs comprise one or more variants found in complement component 2 (C2), complement factor B (CFB), complement component 3 (C3) and age-related maculopathy susceptibility protein 2 (ARMS2) genes, for example. In some embodiments, SNPs comprise one or more of rs1061170, rs2274700, rs403846, rs12144939, rs1409153, rs1750311, rs10922153, rs698859, rs2990510, rs9332739, rs641153, rs10490924, rs2230199, rs11200638, rs1061147, rs1329422, rs2300430, rs10801553, rs1329421, rs10801554, rs7529589, rs1329424, rs572515, rs10922152, rs203674, rs393955, rs381974, rs395544, rs3800390, rs3748557, rs12755054, rs1759016, and rs4151667. In some embodiments, SNPs comprise one or more of rs1061170, rs2274700, rs403846, rs12144939, rs1409153, rs1750311, rs10922153, rs698859, rs2990510, rs9332739, rs641153, rs10490924, and rs2230199. In some embodiments, SNPs comprise one or more of rs10490924, rs1061170, rs10922153, rs403846, and rs1750311. In some embodiments, SNPs comprise one or more of rs10490924 and rs641153. In some embodiments, a SNP is rs10490924. In some embodiments, a SNP is rs641153. The following table presents examples of SNPs that can be utilized and their corresponding genes.

| Gene | SNPs |
|---|---|
| ARMS2 | rs10490924 |
| | rs11200638 |
| CFH | rs403846 |
| | rs12144939 |
| | rs1061170 |
| | rs2274700 |
| | rs1061147 |
| | rs1329422 |
| | rs2300430 |
| | rs10801553 |
| | rs1329421 |
| | rs10801554 |
| | rs7529589 |
| | rs1329424 |
| | rs572515 |
| | rs203674 |
| | rs393955 |
| | rs381974 |
| | rs395544 |
| | rs3800390 |
| C3 | rs2230199 |
| F13B | rs698859 |
| | rs2990510 |
| CFHR4 | rs1409153 |
| CFHR5 | rs1750311 |
| | rs10922153 |
| | rs3748557 |
| | rs12755054 |
| | rs1759016 |
| | rs10922152 |
| CFB | rs641153 |
| | rs4151667 |
| C2 | rs9332739 |

### Non-Genetic Factors

In some embodiments, methods for predicting a therapeutic effect and/or predicting a disease subtype include an assessment of one or more non-genetic factors. Non-genetic factors may include environmental, lifestyle and/or behavioral factors. For example, non-genetic factors may include stress, physical and mental abuse, diet, exposure to toxins, pathogens, radiation and chemicals, oxidative stress, alcohol use, prescription drug use, recreational drug use, smoking, physical activity, sleep, weight, BMI, healthcare, blood pressure, cholesterol level, diabetes, and/or sun exposure. In some instances, a non-genetic factor includes smoking status (e.g., never smoked, smoked in the past, currently smoke). Smoking status sometimes may depend on the amount of tobacco smoked in a given time period. Other non-genetic factors may include age, race and/or menopausal state. In some embodiments, methods for predicting a therapeutic effect and/or predicting a disease subtype do not include an assessment of one or more non-genetic factors.

### Nucleic Acids

A genotype or other genetic assessment can be obtained by analyzing particular polynucleotides within a nucleic acid. Target or sample nucleic acid may be derived from one or more samples or sources. “Sample nucleic acid” as used herein refers to a nucleic acid from a sample. “Target nucleic acid” and “template nucleic acid” are used interchangeably throughout the document and refer to a nucleic acid of interest. Target nucleic acid may comprise one or more genetic markers, in some embodiments, such as polymorphic loci (e.g., SNPs). The terms “total nucleic acid” or “nucleic acid composition” as used herein, refer to the entire population of nucleic acid species from or in a sample or source. Non-limiting examples of nucleic acid compositions containing “total nucleic acids” include, host and non-host nucleic acid, maternal and fetal nucleic acid, genomic and acellular nucleic acid, or mixed-population nucleic acids isolated from environmental sources. As used herein, “nucleic acid” refers to polynucleotides such as deoxyribonucleic acid (DNA) and ribonucleic acid (RNA), and refers to derivatives, variants and analogs of RNA or DNA made from nucleotide analogs, single (sense or antisense) and double-stranded polynucleotides. The term “nucleic acid” does not refer to or infer a specific length of the polynucleotide chain, thus nucleotides, polynucleotides, and oligonucleotides are also included within “nucleic acid.”

A sample containing nucleic acids may be collected from an organism, mineral or geological site (e.g., soil, rock, mineral deposit, combat theater), forensic site (e.g., crime scene, contraband or suspected contraband), or a paleontological or archeological site (e.g., fossil, or bone), for example. A sample may be a “biological sample,” which refers to any material obtained from a living source or formerly-living source, for example, an animal such as a human or other mammal, a plant, a bacterium, a fungus, a protist or a virus. Template or sample nucleic acid utilized in methods and kits described herein often is obtained and isolated from a subject. A subject can be any living or non-living source, including but not limited to a human, an animal, a plant, a bacterium, a fungus, a protist. Any human or animal can be selected, including but not limited to, non-human, mammal, reptile, cattle, cat, dog, goat, swine, pig, monkey, ape, gorilla, bull, cow, bear, horse, sheep, poultry, mouse, rat, fish, dolphin, whale, and shark, or any animal or organism that may have a detectable genetic abnormality. The sample may be heterogeneous, by which is meant that more than one type of nucleic acid species is present in the sample. A sample may be heterogeneous because more than one cell type is present, such as a fetal cell and a maternal cell or a cancer and non-cancer cell.

The biological or subject sample can be in any form, including without limitation umbilical cord blood, chorionic villi, amniotic fluid, cerebrospinal fluid, spinal fluid, lavage fluid (e.g., bronchioalveolar, gastric, peritoneal, ductal, ear, arthroscopic), exudate from a region of infection or inflammation, or a mouth wash containing buccal cells, biopsy sample (e.g., from pre-implantation embryo), celocentesis sample, fetal nucleated cells or fetal cellular remnants, washings of female reproductive tract, urine, feces, sputum, saliva, nasal mucous, prostate fluid, lavage, semen, lymphatic fluid, bile, tears, sweat, breast milk, breast fluid, embryonic cells and fetal cells, solid material such as tissue, cells, a cell pellet, a cell extract, or a biopsy, or a biological fluid such as urine, blood, saliva, amniotic fluid, urine, cerebral spinal fluid and synovial fluid and organs. In some embodiments, a biological sample may be blood.

As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to further preparation in such embodiments. A fluid or tissue sample from which template nucleic acid is extracted may be acellular. In some embodiments, a fluid or tissue sample may contain cellular elements or cellular remnants.

In some embodiments, the nucleic acid composition containing the target nucleic acid or nucleic acids may be collected from a cell free or substantially cell free biological composition, blood plasma, blood serum or urine for example. The term “substantially cell free” as used herein, refers to biologically derived preparations or compositions that contain a substantially small number of cells, or no cells. A preparation intended to be completely cell free, but containing cells or cell debris can be considered substantially cell free. That is, substantially cell free biological preparations can include up to about 50 cells or fewer per milliliter of preparation (e.g., up to about 50 cells per milliliter or less, 45 cells per milliliter or less, 40 cells per milliliter or less, 35 cells per milliliter or less, 30 cells per milliliter or less, 25 cells per milliliter or less, 20 cells per milliliter or less, 15 cells per milliliter or less, 10 cells per milliliter or less, 5 cells per milliliter or less, or up to about 1 cell per milliliter or less).

Nucleic acid may be derived from one or more sources (e.g., cells, soil, etc.) by methods known in the art. Cell lysis procedures and reagents are commonly known in the art and may generally be performed by chemical, physical, or electrolytic lysis methods. For example, chemical methods generally employ lysing agents to disrupt the cells and extract the nucleic acids from the cells, followed by treatment with chaotropic salts. Physical methods such as freeze/thaw followed by grinding, the use of cell presses and the like are also useful. High salt lysis procedures are also commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, solution 1 can contain 15 mM Tris, pH 8.0; 10 mM EDTA and 100 ug/ml Rnase A; solution 2 can contain 0.2N NaOH and 1% SDS; and solution 3 can contain 3M KOAc, pH 5.5. These procedures can be found in Current Protocols in Molecular Biology, John Wiley & Sons, N.Y., 6.3.1-6.3.6 (1989), incorporated herein in its entirety.

A sample also may be isolated at a different time point as compared to another sample, where each of the samples may be from the same or a different source. A sample nucleic acid may be from a nucleic acid library, such as a cDNA or RNA library, for example. A sample nucleic acid may be a result of nucleic acid purification or isolation and/or amplification of nucleic acid molecules from the sample. Sample nucleic acid provided for sequence analysis processes described herein may contain nucleic acid from one sample or from two or more samples (e.g., from 1, 2, 3, 4, 5, 6, 7, 8, 9, 10, 11, 12, 13, 14, 15, 16, 17, 18, 19 or 20 or more samples).

Sample nucleic acid may comprise or consist essentially of any type of nucleic acid suitable for use with processes of the invention, such as sample nucleic acid that can hybridize to solid phase nucleic acid (described hereafter), for example. A sample nucleic in certain embodiments can comprise or consist essentially of DNA (e.g., complementary DNA (cDNA), genomic DNA (gDNA) and the like), RNA (e.g., message RNA (mRNA), short inhibitory RNA (siRNA), microRNA, ribosomal RNA (rRNA), tRNA and the like), and/or DNA or RNA analogs (e.g., containing base analogs, sugar analogs and/or a non-native backbone and the like). A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, a cell, a cell nucleus or cytoplasm of a cell in certain embodiments. A sample nucleic acid in some embodiments is from a single chromosome (e.g., a nucleic acid sample may be from one chromosome of a sample obtained from a diploid organism). Deoxyribonucleotides include deoxyadenosine, deoxycytidine, deoxyguanosine and deoxythymidine. For RNA, the uracil base is uridine. A source or sample containing sample nucleic acid(s) may contain one or a plurality of sample nucleic acids. A plurality of sample nucleic acids as described herein refers to at least 2 sample nucleic acids and includes nucleic acid sequences that may be identical or different. That is, the sample nucleic acids may all be representative of the same nucleic acid sequence, or may be representative of two or more different nucleic acid sequences (e.g., from 1, 2, 3, 4, 5, 6, 7, 8, 9, 10, 11, 12, 13, 14, 15, 16, 17, 18, 19, 20, 50, 100, 1000 or more sequences).

Sample or template nucleic acid can include different nucleic acid species, including extracellular nucleic acid, and therefore is referred to herein as “heterogeneous” in certain embodiments. For example, blood serum or plasma from a person having cancer can include nucleic acid from cancer cells and nucleic acid from non-cancer cells. The term “extracellular template or sample nucleic acid” as used herein refers to nucleic acid isolated from a source having substantially no cells (e.g., no detectable cells, or fewer than 50 cells per milliliter or less as described above, or may contain cellular elements or cellular remnants). Examples of acellular sources for extracellular nucleic acid are blood plasma, blood serum and urine. Without being limited by theory, extracellular nucleic acid may be a product of cell apoptosis and cell breakdown, which provides basis for extracellular nucleic acid often having a series of lengths across a large spectrum (e.g., a “ladder”). In some embodiments, the nucleic acids can be cell free nucleic acid.

The term “nucleotides”, as used herein, in reference to the length of nucleic acid chain, refers to a single stranded nucleic acid chain. The term “base pairs”, as used herein, in reference to the length of nucleic acid chain, refers to a double stranded nucleic acid chain.

Sample nucleic acid may be provided for conducting methods described herein without processing of the sample(s) containing the nucleic acid in certain embodiments. In some embodiments, sample nucleic acid is provided for conducting methods described herein after processing of the sample(s) containing the nucleic acid. For example, a sample nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to sample nucleic acid provided that contains fewer nucleic acid species than in the sample source from which the sample nucleic acid is derived. A composition comprising sample nucleic acid may be substantially purified (e.g., about 90%, 91%, 92%, 93%, 94%, 95%, 96%, 97%, 98%, 99% or greater than 99% free of other nucleic acid species). The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

Sample nucleic acid also may be processed by subjecting nucleic acid to a method that generates nucleic acid fragments, in certain embodiments, before providing sample nucleic acid for a process described herein. In some embodiments, sample nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 base pairs. Fragments can be generated by any suitable method known in the art, and the average, mean or nominal length of nucleic acid fragments can be controlled by selecting an appropriate fragment-generating procedure. In certain embodiments, sample nucleic acid of a relatively shorter length can be utilized to analyze sequences that contain little sequence variation and/or contain relatively large amounts of known nucleotide sequence information. In some embodiments, sample nucleic acid of a relatively longer length can be utilized to analyze sequences that contain greater sequence variation and/or contain relatively small amounts of unknown nucleotide sequence information.

Sample nucleic acid fragments can contain overlapping nucleotide sequences, and such overlapping sequences can facilitate construction of a nucleotide sequence of the previously non-fragmented sample nucleic acid, or a portion thereof. For example, one fragment may have subsequences x and y and another fragment may have subsequences y and z, where x, y and z are nucleotide sequences that can be 5 nucleotides in length or greater. Overlap sequence y can be utilized to facilitate construction of the x-y-z nucleotide sequence in nucleic acid from a sample in certain embodiments. Sample nucleic acid may be partially fragmented (e.g., from an incomplete or terminated specific cleavage reaction) or fully fragmented in certain embodiments.

Sample nucleic acid can be fragmented by various methods known in the art, which include without limitation, physical, chemical and enzymatic processes. Examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected to generate non-specifically cleaved fragments or specifically cleaved fragments. Examples of processes that can generate non-specifically cleaved fragment sample nucleic acid include, without limitation, contacting sample nucleic acid with apparatus that expose nucleic acid to shearing force (e.g., passing nucleic acid through a syringe needle; use of a French press); exposing sample nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

Sample nucleic acid may be specifically cleaved by contacting the nucleic acid with one or more specific cleavage agents. The term “specific cleavage agent” as used herein refers to an agent, sometimes a chemical or an enzyme that can cleave a nucleic acid at one or more specific sites. Specific cleavage agents often will cleave specifically according to a particular nucleotide sequence at a particular site.

Examples of enzymic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, Cla I, Dde I, Dpn I, Dra I, EcIX I, EcoR I, EcoR I, EcoR II, EcoR V, Hae II, Hae II, Hind III, Hind III, Hpa I, Hpa II, Kpn I, Ksp I, Mlu I, MluN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I.); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

As used herein, the term “complementary cleavage reactions” refers to cleavage reactions that are carried out on the same sample nucleic acid using different cleavage reagents or by altering the cleavage specificity of the same cleavage reagent such that alternate cleavage patterns of the same target or reference nucleic acid or protein are generated. In certain embodiments, sample nucleic acid may be treated with one or more specific cleavage agents (e.g., 1, 2, 3, 4, 5, 6, 7, 8, 9, 10 or more specific cleavage agents) in one or more reaction vessels (e.g., sample nucleic acid is treated with each specific cleavage agent in a separate vessel).

Sample nucleic acid also may be exposed to a process that modifies certain nucleotides in the nucleic acid before providing sample nucleic acid for a method described herein. A process that selectively modifies nucleic acid based upon the methylation state of nucleotides therein can be applied to sample nucleic acid, for example. The term “methylation state” as used herein refers to whether a particular nucleotide in a polynucleotide sequence is methylated or not methylated. Methods for modifying a target nucleic acid molecule in a manner that reflects the methylation pattern of the target nucleic acid molecule are known in the art, as exemplified in U.S. Pat. No. 5,786,146 and U.S. patent publications 20030180779 and 20030082600. For example, non-methylated cytosine nucleotides in a nucleic acid can be converted to uracil by bisulfite treatment, which does not modify methylated cytosine. Non-limiting examples of agents that can modify a nucleotide sequence of a nucleic acid include methylmethane sulfonate, ethylmethane sulfonate, diethylsulfate, nitrosoguanidine (N-methyl-N′-nitro-N-nitrosoguanidine), nitrous acid, di-(2-chloroethyl)sulfide, di-(2-chloroethyl)methylamine, 2-aminopurine, t-bromouracil, hydroxylamine, sodium bisulfite, hydrazine, formic acid, sodium nitrite, and 5-methylcytosine DNA glycosylase. In addition, conditions such as high temperature, ultraviolet radiation, x-radiation, can induce changes in the sequence of a nucleic acid molecule.

Sample nucleic acid may be provided in any form useful for conducting a method described herein, such as solid or liquid form, for example. In certain embodiments, sample nucleic acid may be provided in a liquid form optionally comprising one or more other components, including without limitation one or more buffers or salts selected.

### Nucleic Acid Analysis

Genetic markers (e.g., single nucleotide polymorphisms (SNPs)) and genotypes at such markers can be identified through analysis of the nucleic acid sequence present at one or more of the polymorphic sites using methods known in the art. Such methods can include hybridization, for example with a probe (e.g., probe-based methods). Some methods, for example, can involve amplification of nucleic acid (e.g., amplification-based methods). In some embodiments, methods can include both hybridization and amplification. In some embodiments, genetic markers can be identified by querying a database comprising all or part of an individual's genome for one or more specific markers (e.g., SNPs) in the genetic profile.

Amplification

In some embodiments, one or more nucleic acids are amplified using a suitable amplification process. It may be desirable to amplify a nucleic acid particularly if one or more of the nucleic acid exists at low copy number. In some embodiments amplification of sequences or regions of interest may aid in detection of polymorphisms. An amplification product (amplicon) of a particular nucleic acid is referred to herein as an “amplified nucleic acid.”

Nucleic acid amplification often involves enzymatic synthesis of nucleic acid amplicons (copies), which contain a sequence complementary to a nucleic acid being amplified. Amplifying nucleic acid and detecting the amplicons synthesized, can improve the sensitivity of an assay, since fewer target sequences are needed at the beginning of the assay, and can improve detection of a nucleic acid.

Any suitable amplification technique can be utilized. Amplification of polynucleotides include, but are not limited to, polymerase chain reaction (PCR); ligation amplification (or ligase chain reaction (LCR)); amplification methods based on the use of Q-beta replicase or template-dependent polymerase (see US Patent Publication Number US20050287592); helicase-dependant isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR (i.e., amplification primers and/or conditions selected that generate a product when a polymorphism of interest is present), Alu-PCR, Asymmetric PCR, Colony PCR, digital PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, combinations thereof, and the like. Reagents and hardware for conducting PCR are commercially available.

The terms “amplify”, “amplification”, “amplification reaction”, or “amplifying” refers to any in vitro processes for multiplying the copies of a target sequence of nucleic acid. Amplification sometimes refers to an “exponential” increase in target nucleic acid. However, “amplifying” as used herein can also refer to linear increases in the numbers of a select target sequence of nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions, and also may reduce amplification biases due to nucleotide sequence or species abundance of the target. In some embodiments a one-time primer extension may be used may be performed as a prelude to linear or exponential amplification.

A generalized description of an amplification process is presented herein. Primers and target nucleic acid are contacted, and complementary sequences anneal to one another, for example. Primers can anneal to a target nucleic acid, at or near (e.g., adjacent to, abutting, and the like) a sequence of interest. A reaction mixture, containing components necessary for enzymatic functionality, is added to the primer-target nucleic acid hybrid, and amplification can occur under suitable conditions. Components of an amplification reaction may include, but are not limited to, e.g., primers (e.g., individual primers, primer pairs, primer sets and the like) a polynucleotide template (e.g., target nucleic acid), polymerase, nucleotides, dNTPs and the like. In some embodiments, non-naturally occurring nucleotides or nucleotide analogs, such as analogs containing a detectable label (e.g., fluorescent or colorimetric label), may be used for example. Polymerases can be selected and include polymerases for thermocycle amplification (e.g., Taq DNA Polymerase; Q-Bio™ Taq DNA Polymerase (recombinant truncated form of Taq DNA Polymerase lacking 5′-3′ exo activity); SurePrime™ Polymerase (chemically modified Taq DNA polymerase for “hot start” PCR); Arrow™ Taq DNA Polymerase (high sensitivity and long template amplification)) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

The terms “near” or “adjacent to” when referring to a nucleotide sequence of interest refers to a distance or region between the end of the primer and the nucleotide or nucleotides of interest. As used herein adjacent is in the range of about 5 nucleotides to about 500 nucleotides (e.g., about 5 nucleotides away from nucleotide of interest, about 10, about 20, about 30, about 40, about 50, about 60, about 70, about 80, about 90, about 100, about 150, about 200, about 250, about 300, abut 350, about 400, about 450 or about 500 nucleotides from a nucleotide of interest). In some embodiments the primers in a set hybridize within about 10 to 30 nucleotides from a nucleic acid sequence of interest and produce amplified products.

Each amplified nucleic acid independently is about 10 to about 500 base pairs in length in some embodiments. In certain embodiments, an amplified nucleic acid is about 20 to about 250 base pairs in length, sometimes is about 50 to about 150 base pairs in length and sometimes is about 100 base pairs in length. Thus, in some embodiments, the length of each of the amplified nucleic acid products independently is about 10, 15, 20, 25, 30, 35, 40, 45, 50, 55, 60, 65, 70, 75, 80, 82, 84, 86, 88, 90, 92, 94, 96, 98, 100, 102, 104, 106, 108, 110, 112, 114, 116, 118, 120, 125, 130, 135, 140, 145, 150, 175, 200, 250, 300, 350, 400, 450, or 500 base pairs (bp) in length.

An amplification product may include naturally occurring nucleotides, non-naturally occurring nucleotides, nucleotide analogs and the like and combinations of the foregoing. An amplification product often has a nucleotide sequence that is identical to or substantially identical to a sample nucleic acid nucleotide sequence or complement thereof. A “substantially identical” nucleotide sequence in an amplification product will generally have a high degree of sequence identity to the nucleic acid being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of infidelity of the polymerase used for extension and/or amplification, or additional nucleotide sequence(s) added to the primers used for amplification.

PCR conditions can be dependent upon primer sequences, target abundance, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds, 1990. Digital PCR is also known to those of skill in the art; see, e.g., US Patent Application Publication Number 20070202525, filed Feb. 2, 2007, which is hereby incorporated by reference). PCR often is carried out as an automated process with a thermostable enzyme. In this process, the temperature of the reaction mixture is cycled through a denaturing region, a primer-annealing region, and an extension reaction region automatically. Machines specifically adapted for this purpose are commercially available. A non-limiting example of a PCR protocol that may be suitable for embodiments described herein is, treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. Suitable isothermal amplification processes known and selected also may be applied, in certain embodiments.

In some embodiments, multiplex amplification processes may be used to amplify target nucleic acids, such that multiple amplicons are simultaneously amplified in a single, homogenous reaction. As used herein “multiplex amplification” refers to a variant of PCR where simultaneous amplification of many targets of interest in one reaction vessel may be accomplished by using more than one pair of primers (e.g., more than one primer set). Multiplex amplification may be useful for analysis of deletions, mutations, and polymorphisms, or quantitative assays, in some embodiments. In certain embodiments multiplex amplification may be used for detecting paralog sequence imbalance, genotyping applications where simultaneous analysis of multiple markers is required, detection of pathogens or genetically modified organisms, or for microsatellite analyses. In some embodiments multiplex amplification may be combined with another amplification (e.g., PCR) method (e.g., digital PCR, nested PCR or hot start PCR, for example) to increase amplification specificity and reproducibility. In other embodiments multiplex amplification may be done in replicates, for example, to reduce the variance introduced by said amplification.

In certain embodiments, nucleic acid amplification can generate additional nucleic acid species of different or substantially similar nucleic acid sequence. In certain embodiments described herein, contaminating or additional nucleic acid species, which may contain sequences substantially complementary to, or may be substantially identical to, the sequence of interest, can be useful for sequence quantification, with the proviso that the level of contaminating or additional sequences remains constant and therefore can be a reliable marker whose level can be substantially reproduced. Additional considerations that may affect sequence amplification reproducibility are: PCR conditions (number of cycles, volume of reactions, melting temperature difference between primers pairs, and the like), concentration of target nucleic acid in sample, the number of chromosomes on which the nucleotide species of interest resides, variations in quality of prepared sample, and the like. The terms “substantially reproduced” or “substantially reproducible” as used herein refer to a result (e.g., quantifiable amount of nucleic acid) that under substantially similar conditions would occur in substantially the same way about 75% of the time or greater, about 80%, about 85%, about 90%, about 95%, or about 99% of the time or greater.

In some embodiments where a target nucleic acid is RNA, prior to the amplification step, a DNA copy (cDNA) of the RNA transcript of interest may be synthesized. A cDNA can be synthesized by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, Minn., 1993; Egger et al., J. Clin. Microbiol. 33:1442-1447, 1995; and U.S. Pat. No. 5,075,212. Branched-DNA technology may be used to amplify the signal of RNA markers in maternal blood. For a review of branched-DNA (bDNA) signal amplification for direct quantification of nucleic acid sequences in clinical samples, see Nolte, Adv. Clin. Chem. 33:201-235, 1998.

Amplification also can be accomplished using digital PCR, in certain embodiments (e.g., Kalinina and colleagues (Kalinina et al., “Nanoliter scale PCR with TaqMan detection.” Nucleic Acids Research. 25; 1999-2004, (1997); Vogelstein and Kinzler (Digital PCR. Proc Natl Acad Sci USA. 96; 9236-41, (1999); PCT Patent Publication No. WO05023091A2; US Patent Publication No. US 20070202525). Digital PCR takes advantage of nucleic acid (DNA, cDNA or RNA) amplification on a single molecule level, and offers a highly sensitive method for quantifying low copy number nucleic acid. Systems for digital amplification and analysis of nucleic acids are available (e.g., Fluidigm® Corporation). Digital PCR is useful for studying variations in gene sequences (e.g., copy number variants, point mutations, and the like). In general, samples being analyzed by digital PCR are partitioned (e.g., captured, isolated) into reaction vessels or chambers such that a single nucleic acid is contained in each reaction, in some embodiments. Samples can be partitioned using any method known in the art, non-limiting examples of which include the use of micro well plates (e.g., microtiter plates) capillaries, the dispersed phase of an emulsion, microfluidic devices, solid supports, the like or combinations of the foregoing. Partitioning of the sample allows estimation of the number of molecules according to Poisson distribution. Generally, each reaction vessel will contain 0 or 1 starting nucleic acid molecules from which amplification occurs. Reactions with 0 nucleic acid molecules do no generate an amplified product, whereas reactions with 1 nucleic acid generate an amplified product. After amplification, nucleic acids may be quantified by counting the reactions that generate a PCR product. Digital PCR generally does not rely on the number of amplification cycles performed to determine the number of copies of a nucleic acid of interest in a sample. Thus, digital PCR reduces or eliminates reliance on data from procedures that use exponential amplification, which sometimes can introduce amplification artifacts. Digital PCR generally provides a more robust method of quantification than conventional PCR.

In some embodiments, digital PCR is performed with primer sets that include one or more primers that anneal to nucleic acid sequences located within a multiplied region (e.g., a multiplied CFH allele or CFHR allele). In certain embodiments, digital PCR is performed with primer sets that include one or more primers that anneal to nucleic acid sequences located within a multiplied region and/or one or more primers that anneal to nucleic acid sequences located outside of a multiplied region. In some embodiments, a primer set includes one or more primers that amplify a control region, which control region does not include a multiplied region. In some embodiments, one or more primers utilized in a digital PCR assay described herein includes a polymorphic nucleotide position, and in certain embodiments, the polymorphic nucleotide position is determinative of the presence or absence of a haplotype associated with a disease condition. In some embodiments, a haplotype is associated with a polymorphic nucleotide, a multiplied region or a polymorphic nucleotide and a multiplied region. In some embodiments, the disease condition is AMD.

Use of a primer extension reaction also can be applied in methods of the technology. A primer extension reaction operates, for example, by discriminating nucleic acid sequences at a single nucleotide mismatch, in some embodiments. The mismatch is detected by the incorporation of one or more deoxynucleotides and/or dideoxynucleotides to an extension oligonucleotide, which hybridizes to a region adjacent to the mismatch site. The extension oligonucleotide generally is extended with a polymerase. In some embodiments, a detectable tag or detectable label is incorporated into the extension oligonucleotide or into the nucleotides added on to the extension oligonucleotide (e.g., biotin or streptavidin). The extended oligonucleotide can be detected by any known suitable detection process (e.g., mass spectrometry; sequencing processes). In some embodiments, the mismatch site is extended only by one or two complementary deoxynucleotides or dideoxynucleotides that are tagged by a specific label or generate a primer extension product with a specific mass, and the mismatch can be discriminated and quantified.

In some embodiments, amplification may be performed on a solid support. In some embodiments, primers may be associated with a solid support. In certain embodiments, target nucleic acid (e.g., template nucleic acid) may be associated with a solid support. A nucleic acid (primer or target) in association with a solid support often is referred to as a solid phase nucleic acid.

In some embodiments, nucleic acid molecules provided for amplification and in a “microreactor”. As used herein, the term “microreactor” refers to a partitioned space in which a nucleic acid molecule can hybridize to a solid support nucleic acid molecule. Examples of microreactors include, without limitation, an emulsion globule (described hereafter) and a void in a substrate. A void in a substrate can be a pit, a pore or a well (e.g., microwell, nanowell, picowell, micropore, or nanopore) in a substrate constructed from a solid material useful for containing fluids (e.g., plastic (e.g., polypropylene, polyethylene, polystyrene) or silicon) in certain embodiments. Emulsion globules are partitioned by an immiscible phase as described in greater detail hereafter. In some embodiments, the microreactor volume is large enough to accommodate one solid support (e.g., bead) in the microreactor and small enough to exclude the presence of two or more solid supports in the microreactor.

The term “emulsion” as used herein refers to a mixture of two immiscible and unblendable substances, in which one substance (the dispersed phase) often is dispersed in the other substance (the continuous phase). The dispersed phase can be an aqueous solution (i.e., a solution comprising water) in certain embodiments. In some embodiments, the dispersed phase is composed predominantly of water (e.g., greater than 70%, greater than 75%, greater than 80%, greater than 85%, greater than 90%, greater than 95%, greater than 97%, greater than 98% and greater than 99% water (by weight)). Each discrete portion of a dispersed phase, such as an aqueous dispersed phase, is referred to herein as a “globule” or “microreactor.” A globule sometimes may be spheroidal, substantially spheroidal or semi-spheroidal in shape, in certain embodiments.

The terms “emulsion apparatus” and “emulsion component(s)” as used herein refer to apparatus and components that can be used to prepare an emulsion. Non-limiting examples of emulsion apparatus include without limitation counter-flow, cross-current, rotating drum and membrane apparatus suitable for use to prepare an emulsion. An emulsion component forms the continuous phase of an emulsion in certain embodiments, and includes without limitation a substance immiscible with water, such as a component comprising or consisting essentially of an oil (e.g., a heat-stable, biocompatible oil (e.g., light mineral oil)). A biocompatible emulsion stabilizer can be utilized as an emulsion component. Emulsion stabilizers include without limitation Atlox 4912, Span 80 and other biocompatible surfactants.

In some embodiments, components useful for biological reactions can be included in the dispersed phase. Globules of the emulsion can include (i) a solid support unit (e.g., one bead or one particle); (ii) sample nucleic acid molecule; and (iii) a sufficient amount of extension agents to elongate solid phase nucleic acid and amplify the elongated solid phase nucleic acid (e.g., extension nucleotides, polymerase, primer). Inactive globules in the emulsion may include a subset of these components (e.g., solid support and extension reagents and no sample nucleic acid) and some can be empty (i.e., some globules will include no solid support, no sample nucleic acid and no extension agents).

Emulsions may be prepared using known suitable methods (e.g., Nakano et al. “Single-molecule PCR using water-in-oil emulsion;” Journal of Biotechnology 102 (2003) 117-124). Emulsification methods include without limitation adjuvant methods, counter-flow methods, cross-current methods, rotating drum methods, membrane methods, and the like. In certain embodiments, an aqueous reaction mixture containing a solid support (hereafter the “reaction mixture”) is prepared and then added to a biocompatible oil. In certain embodiments, the reaction mixture may be added dropwise into a spinning mixture of biocompatible oil (e.g., light mineral oil (Sigma)) and allowed to emulsify. In some embodiments, the reaction mixture may be added dropwise into a cross-flow of biocompatible oil. The size of aqueous globules in the emulsion can be adjusted, such as by varying the flow rate and speed at which the components are added to one another, for example.

The size of emulsion globules can be selected in certain embodiments based on two competing factors: (i) globules are sufficiently large to encompass one solid support molecule, one sample nucleic acid molecule, and sufficient extension agents for the degree of elongation and amplification required; and (ii) globules are sufficiently small so that a population of globules can be amplified by conventional laboratory equipment (e.g., thermocycling equipment, test tubes, incubators and the like). Globules in the emulsion can have a nominal, mean or average diameter of about 5 microns to about 500 microns, about 10 microns to about 350 microns, about 50 to 250 microns, about 100 microns to about 200 microns, or about 5, 10, 15, 20, 25, 30, 35, 40, 45, 50, 55, 60, 65, 70, 75, 80, 85, 90, 95, 100, 200, 300, 400 or 500 microns in certain embodiments.

Primers

Primers useful for detection, quantification, amplification, sequencing and analysis of nucleic acid are provided. In some embodiments primers are used in sets, where a set contains at least a pair. In some embodiments a set of primers may include a third or a fourth nucleic acid (e.g., two pairs of primers or nested sets of primers, for example). A plurality of primer pairs may constitute a primer set in certain embodiments (e.g., about 2, 3, 4, 5, 6, 7, 8, 9, 10, 15, 20, 25, 30, 35, 40, 45, 50, 55, 60, 65, 70, 75, 80, 85, 90, 95 or 100 pairs). In some embodiments a plurality of primer sets, each set comprising pair(s) of primers, may be used. The term “primer” as used herein refers to a nucleic acid that comprises a nucleotide sequence capable of hybridizing or annealing to a target nucleic acid, at or near (e.g., adjacent to) a specific region of interest (e.g., polymorphism). Primers can allow for specific determination of a target nucleic acid nucleotide sequence or detection of the target nucleic acid (e.g., presence or absence of a sequence or copy number of a sequence), or feature thereof, such as a polymorphism, for example. A primer may be naturally occurring or synthetic. The term “specific” or “specificity”, as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “primer”, “oligo”, or “oligonucleotide” may be used interchangeably throughout the document, when referring to primers.

A primer nucleic acid can be designed and synthesized using suitable processes, and may be of any length suitable for hybridizing to a nucleotide sequence of interest (e.g., where the nucleic acid is in liquid phase or bound to a solid support) and performing analysis processes described herein. Primers may be designed based upon a target nucleotide sequence. A primer in some embodiments may be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer may be composed of naturally occurring and/or non-naturally occurring nucleotides (e.g., labeled nucleotides), or a mixture thereof. Primers suitable for use with embodiments described herein, may be synthesized and labeled using known techniques. Oligonucleotides (e.g., primers) may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides can be effected by native acrylamide gel electrophoresis or by anion-exchange high-performance liquid chromatography (HPLC), for example, as described in Pearson and Regnier, J. Chrom., 255:137-149, 1983.

All or a portion of a primer nucleic acid sequence (naturally occurring or synthetic) may be substantially complementary to a target nucleic acid, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are regions of counterpart, target and capture nucleotide sequences 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Primers that are substantially complimentary to a target nucleic acid sequence are also substantially identical to the compliment of the target nucleic acid sequence. That is, primers are substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

Primer sequences and length may affect hybridization to target nucleic acid sequences. Depending on the degree of mismatch between the primer and target nucleic acid, low, medium or high stringency conditions may be used to effect primer/target annealing. As used herein, the term “stringent conditions” refers to conditions for hybridization and washing. Methods for hybridization reaction temperature condition optimization are known to those of skill in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, to a nucleic acid molecule having a sequence complementary to the primer compared to hybridization to a nucleic acid molecule not having a complementary sequence. For example, specific hybridization includes the hybridization of a primer to a target nucleic acid sequence that is complementary to the primer.

In some embodiments primers can include a nucleotide subsequence that may be complementary to a solid phase nucleic acid primer hybridization sequence or substantially complementary to a solid phase nucleic acid primer hybridization sequence (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% identical to the primer hybridization sequence complement when aligned). A primer may contain a nucleotide subsequence not complementary to or not substantially complementary to a solid phase nucleic acid primer hybridization sequence (e.g., at the 3′ or 5′ end of the nucleotide subsequence in the primer complementary to or substantially complementary to the solid phase primer hybridization sequence).

A primer, in certain embodiments, may contain a modification such as inosines, abasic sites, locked nucleic acids, minor groove binders, duplex stabilizers (e.g., acridine, spermidine), Tm modifiers or any modifier that changes the binding properties of the primers or probes.

A primer, in certain embodiments, may contain a detectable molecule or entity (e.g., a fluorophore, radioisotope, colorimetric agent, particle, enzyme and the like). When desired, the nucleic acid can be modified to include a detectable label using any method known to one of skill in the art. The label may be incorporated as part of the synthesis, or added on prior to using the primer in any of the processes described herein. Incorporation of label may be performed either in liquid phase or on solid phase. In some embodiments the detectable label may be useful for detection of targets. Any detectable label suitable for detection of an interaction or biological activity in a system can be appropriately selected and utilized by the artisan. Examples of detectable labels are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., 125I, 131I, 35S, 31P, 32P, 33P, 14C, 3H, 7Be, 28Mg, 57Co, 65Zn, 67Cu, 68Ge, 82Sr, 83Rb, 95Tc, 96Tc, 103Pd, 109Cd, and 127Xe); light scattering labels (e.g., U.S. Pat. No. 6,214,560, and commercially available from Genicon Sciences Corporation, CA); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and other cofactors or biomolecules such as digoxigenin, strepavidin, biotin (e.g., members of a binding pair such as biotin and avidin for example), affinity capture moieties and the like. In some embodiments a primer may be labeled with an affinity capture moiety. Also included in detectable labels are those labels useful for mass modification for detection with mass spectrometry (e.g., matrix-assisted laser desorption ionization (MALDI) mass spectrometry and electrospray (ES) mass spectrometry).

A primer also may refer to a polynucleotide sequence that hybridizes to a subsequence of a target nucleic acid or another primer and facilitates the detection of a primer, a target nucleic acid or both, as with molecular beacons, for example. The term “molecular beacon” as used herein refers to detectable molecule, where the detectable property of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

In some embodiments a molecular beacon can be a single-stranded oligonucleotide capable of forming a stem-loop structure, where the loop sequence may be complementary to a target nucleic acid sequence of interest and is flanked by short complementary arms that can form a stem. The oligonucleotide may be labeled at one end with a fluorophore and at the other end with a quencher molecule. In the stem-loop conformation, energy from the excited fluorophore is transferred to the quencher, through long-range dipole-dipole coupling similar to that seen in fluorescence resonance energy transfer, or FRET, and released as heat instead of light. When the loop sequence is hybridized to a specific target sequence, the two ends of the molecule are separated and the energy from the excited fluorophore is emitted as light, generating a detectable signal. Molecular beacons offer the added advantage that removal of excess probe is unnecessary due to the self-quenching nature of the unhybridized probe. In some embodiments molecular beacon probes can be designed to either discriminate or tolerate mismatches between the loop and target sequences by modulating the relative strengths of the loop-target hybridization and stem formation. As referred to herein, the term “mismatched nucleotide” or a “mismatch” refers to a nucleotide that is not complementary to the target sequence at that position or positions. A probe may have at least one mismatch, but can also have 2, 3, 4, 5, 6 or 7 or more mismatched nucleotides.

Detection

Nucleic acid (e.g., target nucleic acid), or amplified nucleic acid, or detectable products prepared from the foregoing, can be detected by a suitable detection process. Non-limiting examples of methods of detection include mass detection of mass modified amplicons (e.g., matrix-assisted laser desorption ionization (MALDI) mass spectrometry and electrospray (ES) mass spectrometry), a primer extension method (e.g., iPLEX®; Sequenom, Inc.), direct DNA sequencing, Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, sequencing-by-synthesis analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization using at least one probe, hybridization using at least one fluorescently labeled probe, in situ hybridization techniques (e.g., fluorescence in situ hybridization (FISH), including fiber FISH), cloning and sequencing, electrophoresis, the use of hybridization probes and quantitative real time polymerase chain reaction (QRT-PCR), digital PCR, nanopore sequencing, chips and combinations thereof. The detection genetic markers can be carried out using the “closed-tube” methods described in U.S. patent application Ser. No. 11/950,395, which was filed Dec. 4, 2007.

A target nucleic acid can be detected by detecting a detectable label or “signal-generating moiety” in some embodiments. The term “signal-generating” as used herein refers to any atom or molecule that can provide a detectable or quantifiable effect, and that can be attached to a nucleic acid. In certain embodiments, a detectable label generates a unique light signal, a fluorescent signal, a luminescent signal, an electrical property, a chemical property, a magnetic property and the like.

Detectable labels include, but are not limited to, nucleotides (labeled or unlabelled), compomers, sugars, peptides, proteins, antibodies, chemical compounds, conducting polymers, binding moieties such as biotin, mass tags, colorimetric agents, light emitting agents, chemiluminescent agents, light scattering agents, fluorescent tags, radioactive tags, charge tags (electrical or magnetic charge), volatile tags and hydrophobic tags, biomolecules (e.g., members of a binding pair antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isothiocyanate, amine/succinimidyl ester, and amine/sulfonyl halides) and the like, some of which are further described below. In some embodiments a probe may contain a signal-generating moiety that hybridizes to a target and alters the passage of the target nucleic acid through a nanopore, and can generate a signal when released from the target nucleic acid when it passes through the nanopore (e.g., alters the speed or time through a pore of known size).

In certain embodiments, sample tags (e.g., index sequences) are introduced to distinguish between samples (e.g., from different patients), thereby allowing for the simultaneous testing of multiple samples. For example, sample tags may introduced as part of the extend primers such that extended primers can be associated with a particular sample.

A solution containing amplicons produced by an amplification process, or a solution containing extension products produced by an extension process, can be subjected to further processing. For example, a solution can be contacted with an agent that removes phosphate moieties from free nucleotides that have not been incorporated into an amplicon or extension product. An example of such an agent is a phosphatase (e.g., alkaline phosphatase). Amplicons and extension products also may be associated with a solid phase, may be washed, may be contacted with an agent that removes a terminal phosphate (e.g., exposure to a phosphatase), may be contacted with an agent that removes a terminal nucleotide (e.g., exonuclease), may be contacted with an agent that cleaves (e.g., endonuclease, ribonuclease), and the like.

The term “solid support” or “solid phase” as used herein refers to an insoluble material with which nucleic acid can be associated. Examples of solid supports for use with processes described herein include, without limitation, arrays, beads (e.g., paramagnetic beads, magnetic beads, microbeads, nanobeads) and particles (e.g., microparticles, nanoparticles). Particles or beads having a nominal, average or mean diameter of about 1 nanometer to about 500 micrometers can be utilized, such as those having a nominal, mean or average diameter, for example, of about 10 nanometers to about 100 micrometers; about 100 nanometers to about 100 micrometers; about 1 micrometer to about 100 micrometers; about 10 micrometers to about 50 micrometers; about 1, 5, 10, 15, 20, 25, 30, 35, 40, 45, 50, 55, 60, 65, 70, 75, 80, 85, 90, 95, 100, 200, 300, 400, 500, 600, 700, 800 or 900 nanometers; or about 1, 5, 10, 15, 20, 25, 30, 35, 40, 45, 50, 55, 60, 65, 70, 75, 80, 85, 90, 95, 100, 200, 300, 400, 500 micrometers.

A solid support can comprise virtually any insoluble or solid material, and often a solid support composition is selected that is insoluble in water. For example, a solid support can comprise or consist essentially of silica gel, glass (e.g. controlled-pore glass (CPG)), nylon, Sephadex®, Sepharose®, cellulose, a metal surface (e.g. steel, gold, silver, aluminum, silicon and copper), a magnetic material, a plastic material (e.g., polyethylene, polypropylene, polyamide, polyester, polyvinylidenedifluoride (PVDF)) and the like. Beads or particles may be swellable (e.g., polymeric beads such as Wang resin) or non-swellable (e.g., CPG). Commercially available examples of beads include without limitation Wang resin, Merrifield resin and Dynabeads® and SoluLink.

A solid support may be provided in a collection of solid supports. A solid support collection comprises two or more different solid support species. The term “solid support species” as used herein refers to a solid support in association with one particular solid phase nucleic acid species or a particular combination of different solid phase nucleic acid species. In certain embodiments, a solid support collection comprises 2 to 10,000 solid support species, 10 to 1,000 solid support species or about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 unique solid support species. The solid supports (e.g., beads) in the collection of solid supports may be homogeneous (e.g., all are Wang resin beads) or heterogeneous (e.g., some are Wang resin beads and some are magnetic beads). Each solid support species in a collection of solid supports sometimes is labeled with a specific identification tag. An identification tag for a particular solid support species sometimes is a nucleic acid (e.g., “solid phase nucleic acid”) having a unique sequence in certain embodiments. An identification tag can be any molecule that is detectable and distinguishable from identification tags on other solid support species.

Nucleic acid, amplified nucleic acid, or detectable products generated from the foregoing may be subject to sequence analysis. The term “sequence analysis” as used herein refers to determining a nucleotide sequence of an amplification product. The entire sequence or a partial sequence of an amplification product can be determined, and the determined nucleotide sequence is referred to herein as a “read.” For example, amplification products may be analyzed directly without further amplification in some embodiments (e.g., by using single-molecule sequencing methodology). In certain embodiments, amplification products may be subject to further amplification and then analyzed (e.g., using sequencing by ligation, sequencing by synthesis, or pyrosequencing methodologies). Reads may be subject to different types of sequence analysis. Any suitable sequencing method can be utilized to analyze (e.g., detect one or more genetic markers) nucleic acid, amplified nucleic acid, or detectable products generated from the foregoing. Examples of certain sequencing methods are described hereafter.

The terms “sequence analysis apparatus” and “sequence analysis component(s)” used herein refer to apparatus, and one or more components used in conjunction with such apparatus, that can be used to determine a nucleotide sequence from amplification products resulting from processes described herein (e.g., linear and/or exponential amplification products) or a non-amplified nucleotide sequence. Examples of sequencing platforms include, without limitation, the 454 platform (Roche) (Margulies, M. et al. 2005 Nature 437, 376-380), Illumina Genomic Analyzer (or Solexa platform), Illumina HISEQ, or SOLID System (Applied Biosystems) or the Helicos True Single Molecule DNA sequencing technology (Harris T D et al. 2008 Science, 320, 106-109), the single molecule, real-time (SMRTTM) technology of Pacific Biosciences, and nanopore sequencing (Soni G V and Meller A. 2007 Clin Chem 53: 1996-2001). Such platforms allow sequencing of many nucleic acid molecules isolated from a specimen at high orders of multiplexing in a parallel manner (Dear Brief Funct Genomic Proteomic 2003; 1: 397-416). Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing. Nucleic acid, amplified nucleic acid and detectable products generated there from can be considered a “study nucleic acid” for purposes of analyzing a nucleotide sequence by such sequence analysis platforms.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label (e.g., 1 fluorescent label, 2, 3, or 4 fluorescent labels).

An example of a system that can be used based on sequencing by ligation generally involves the following steps. Clonal bead populations can be prepared in emulsion microreactors containing study nucleic acid (“template”), amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag. Such a system can be used to exponentially amplify amplification products generated by a process described herein, e.g., by ligating a heterologous nucleic acid to the first amplification product generated by a process described herein and performing emulsion amplification using the same or a different solid support originally used to generate the first amplification product. Such a system also may be used to analyze amplification products directly generated by a process described herein by bypassing an exponential amplification process and directly sorting the solid supports described herein on the glass slide.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphosulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination.

An example of a system that can be used based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslaysky et al., PNAS 100(7): 3960-3964 (2003)). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer-released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In some embodiments, detection of the presence or absence of a multiplied chromosomal region can be performed using fluorescence in situ hybridization (e.g., FISH), and in certain embodiments detection of the presence or absence of a multiplied chromosomal region can be performed using a method referred to as Fiber FISH. FISH is a cytogenetic technique often used to detect and localize the presence or absence of specific DNA sequences on chromosomes. FISH methodology generally makes use of fluorescent probes that bind to only those parts of the chromosome with which they show a high degree of sequence complimentarity. The fluorescent signal typically is visualized utilizing fluorescence microscopy. Fiber FISH is a specialized FISH methodology that makes use of chromatin spreads in which the chromosomes have been mechanically stretched, thereby allowing a higher resolution analysis than conventional FISH. Generally Fiber FISH provides more precise information as to the localization of a specific DNA probe on a chromosome.

Mass spectrometry is a particularly effective method for the detection of nucleic acids (e.g., PCR amplicon, primer extension product, detector probe cleaved from a target nucleic acid). Presence of a target nucleic acid is verified by comparing the mass of the detected signal with the expected mass of the target nucleic acid. The relative signal strength, e.g., mass peak on a spectra, for a particular target nucleic acid indicates the relative population of the target nucleic acid amongst other nucleic acids, thus enabling calculation of a ratio of target to other nucleic acid or sequence copy number directly from the data. For a review of genotyping methods using Sequenom® standard iPLEX® assay and MassARRAY® technology, see Jurinke, C., Oeth, P., van den Boom, D., “MALDI-TOF mass spectrometry: a versatile tool for high-performance DNA analysis.” Mol. Biotechnol. 26, 147-164 (2004). For a review of detecting and quantifying target nucleic using cleavable detector probes that are cleaved during the amplification process and detected by mass spectrometry, see U.S. patent application Ser. No. 11/950,395, which was filed Dec. 4, 2007, and is hereby incorporated by reference. Such approaches may be adapted to detection of genetic targets (e.g., SNPs) by methods described herein.

In some embodiments, a MassARRAY® system (Sequenom, Inc.) can be utilized to perform SNP genotyping in a high-throughput fashion. The MassARRAY® genotyping platform often is complemented by a homogeneous, single-tube assay method (hME or homogeneous MassEXTEND® (Sequenom, Inc.)) in which two genotyping primers anneal to and amplify a genomic target surrounding a polymorphic site of interest. A third primer (the MassEXTEND® primer), which is complementary to the amplified target up to but not including the polymorphism, is enzymatically extended one or a few bases through the polymorphic site and then terminated.

For each polymorphism, a primer set is generated (e.g., a set of PCR primers and a MassEXTEND® primer) to genotype the polymorphism. Primer sets can be generated using any method known in the art. In some embodiments, SpectroDESIGNER™ software (Sequenom, Inc.) is used to design a primer set. A non-limiting example of a PCR amplification scheme suitable for use with a MassARRAY® assay includes a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest and incubation at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) can be added to each reaction (total reaction volume was 7 μl) to remove any residual dNTPs that were not consumed in the PCR step, in some embodiments. Reactions are incubated for 20 minutes at 37° C., followed by 5 minutes at 85° C. to denature the SAP.

After SAP treatment, a primer extension reaction is initiated by adding a polymorphism-specific MassEXTEND® primer cocktail to each sample, in certain embodiments. Each MassEXTEND® cocktail often includes a specific combination of dideoxynucleotides (ddNTPs) and deoxynucleotides (dNTPs) used to distinguish polymorphic alleles from one another. The MassEXTEND® reaction is performed in a total volume of 9 μl, with the addition of 1× ThermoSequenase buffer, 0.576 units of ThermoSequenase (Amersham Pharmacia), 600 nM MassEXTEND® primer, 2 mM of ddATP and/or ddCTP and/or ddGTP and/or ddTTP, and 2 mM of dATP or dCTP or dGTP or dTTP, in some embodiments. The deoxy nucleotide (dNTP) used in the assay generally is complementary to the nucleotide at the polymorphic site in the amplicon. A non-limiting example of reaction conditions for primer extension reactions include incubating reactions at 94° C. for 2 minutes, followed by 55 cycles of 5 seconds at 94° C., 5 seconds at 52° C., and 5 seconds at 72° C.

Following incubation, samples may be desalted by adding 16 μl of water (total reaction volume was 25 μl), 3 mg of SpectroCLEAN™ sample cleaning beads (Sequenom, Inc.) and incubating for 3 minutes with rotation, in some embodiments. For MALDI-TOF analysis, samples can be dispensed onto either 96-spot or 384-spot silicon chips containing a matrix that crystallized each sample (SpectroCHIP® (Sequenom, Inc.)), in certain embodiments. In some embodiments, MALDI-TOF mass spectrometry (Biflex and Autoflex MALDI-TOF mass spectrometers (Bruker Daltonics) can be used) and SpectroTYPER RT™ software (Sequenom, Inc.) can be used to analyze and interpret one or more SNP genotypes for each sample.

Methods provided herein allow for high-throughput detection of nucleic acid in a plurality of nucleic acids (e.g., nucleic acid, amplified nucleic acid and detectable products generated from the foregoing). Multiplexing refers to the simultaneous detection of more than one nucleic acid. General methods for performing multiplexed reactions in conjunction with mass spectrometry, are known (see, e.g., U.S. Pat. Nos. 6,043,031; 5,547,835 and International PCT Application No. WO 97/37041). Multiplexing provides an advantage that a plurality of nucleic acid species (e.g., some having different sequence variations) can be identified in as few as a single mass spectrum, as compared to having to perform a separate mass spectrometry analysis for each individual target nucleic acid species. Methods provided herein lend themselves to high-throughput, highly-automated processes for analyzing sequence variations with high speed and accuracy, in some embodiments. In some embodiments, methods herein may be multiplexed at high levels in a single reaction.

In certain embodiments, the number of nucleic acid species multiplexed include, without limitation, about 1 to about 500 (e.g., about 1-3, 3-5, 5-7, 7-9, 9-11, 11-13, 13-15, 15-17, 17-19, 19-21, 21-23, 23-25, 25-27, 27-29, 29-31, 31-33, 33-35, 35-37, 37-39, 39-41, 41-43, 43-45, 45-47, 47-49, 49-51, 51-53, 53-55, 55-57, 57-59, 59-61, 61-63, 63-65, 65-67, 67-69, 69-71, 71-73, 73-75, 75-77, 77-79, 79-81, 81-83, 83-85, 85-87, 87-89, 89-91, 91-93, 93-95, 95-97, 97-101, 101-103, 103-105, 105-107, 107-109, 109-111, 111-113, 113-115, 115-117, 117-119, 121-123, 123-125, 125-127, 127-129, 129-131, 131-133, 133-135, 135-137, 137-139, 139-141, 141-143, 143-145, 145-147, 147-149, 149-151, 151-153, 153-155, 155-157, 157-159, 159-161, 161-163, 163-165, 165-167, 167-169, 169-171, 171-173, 173-175, 175-177, 177-179, 179-181, 181-183, 183-185, 185-187, 187-189, 189-191, 191-193, 193-195, 195-197, 197-199, 199-201, 201-203, 203-205, 205-207, 207-209, 209-211, 211-213, 213-215, 215-217, 217-219, 219-221, 221-223, 223-225, 225-227, 227-229, 229-231, 231-233, 233-235, 235-237, 237-239, 239-241, 241-243, 243-245, 245-247, 247-249, 249-251, 251-253, 253-255, 255-257, 257-259, 259-261, 261-263, 263-265, 265-267, 267-269, 269-271, 271-273, 273-275, 275-277, 277-279, 279-281, 281-283, 283-285, 285-287, 287-289, 289-291, 291-293, 293-295, 295-297, 297-299, 299-301, 301-303, 303-305, 305-307, 307-309, 309-311, 311-313, 313-315, 315-317, 317-319, 319-321, 321-323, 323-325, 325-327, 327-329, 329-331, 331-333, 333-335, 335-337, 337-339, 339-341, 341-343, 343-345, 345-347, 347-349, 349-351, 351-353, 353-355, 355-357, 357-359, 359-361, 361-363, 363-365, 365-367, 367-369, 369-371, 371-373, 373-375, 375-377, 377-379, 379-381, 381-383, 383-385, 385-387, 387-389, 389-391, 391-393, 393-395, 395-397, 397-401, 401-403, 403-405, 405-407, 407-409, 409-411, 411-413, 413-415, 415-417, 417-419, 419-421, 421-423, 423-425, 425-427, 427-429, 429-431, 431-433, 433-435, 435-437, 437-439, 439-441, 441-443, 443-445, 445-447, 447-449, 449-451, 451-453, 453-455, 455-457, 457-459, 459-461, 461-463, 463-465, 465-467, 467-469, 469-471, 471-473, 473-475, 475-477, 477-479, 479-481, 481-483, 483-485, 485-487, 487-489, 489-491, 491-493, 493-495, 495-497, 497-501).

Design methods for achieving resolved mass spectra with multiplexed assays can include primer and oligonucleotide design methods and reaction design methods. For primer and oligonucleotide design in multiplexed assays, the same general guidelines for primer design applies for uniplexed reactions, such as avoiding false priming and primer dimers, only more primers are involved for multiplex reactions. For mass spectrometry applications, analyte peaks in the mass spectra for one assay are sufficiently resolved from a product of any assay with which that assay is multiplexed, including pausing peaks and any other by-product peaks. Also, analyte peaks optimally fall within a user-specified mass window, for example, within a range of 5,000-8,500 Da. In some embodiments multiplex analysis may be adapted to mass spectrometric detection of chromosome abnormalities, for example. In certain embodiments multiplex analysis may be adapted to various single nucleotide or nanopore based sequencing methods described herein. Commercially produced micro-reaction chambers or devices or arrays or chips may be used to facilitate multiplex analysis, and are commercially available.

### EXAMPLES

The following examples illustrate but do not limit the technology.

### Example 1

### Clinical Validation of a Genetic Model to Estimate the Risk of Developing Choroidal Neovascular Age-Related Macular Degeneration

In this example, the accuracy of a panel of 13 SNPs was assessed without consideration of environmental risk factors such as smoking or BMI, to predict the risk of developing CNV in Caucasian individuals 60 years of age and older. Test model development and validation were designed to evaluate these variants in eight AMD-associated genes (CFH, complement factor H-related 4 (CFHR4), complement factor H-related 5 (CFHR5) and coagulation factor XIII B subunit (F13B) located within the regulators of complement activation (RCA) region on chromosome 1, C2 and CFB on chromosome 6, C3 on chromosome 19 and ARMS2 on chromosome 10. The panel of 13 SNPs was tested in well established case-control and sibling pair cohorts from five academic centers (University of Iowa, University of Utah, Columbia University, Harvard University and Melbourne University) to validate the accuracy of the predictive test and to estimate an individual's genetic risk for developing late-stage CNV. Most of the disease-associated genetic variants in CFH, ARMS2, C2, CFB and C3 were selected based on various criteria including, for example, performance in resolving the most frequent CFH haplotype combinations. Additional SNPs detecting variants in CFHR4 (rs1409153), CFHR5 (rs10922153 and rs1750311) and F13B (rs698859 and rs2990510) tagged additional extended haplotypes spanning the CFH-to-F13B region and were included to maximize the resolution of clinically relevant subtypes suspected to have high association with disease. The inclusion of one or more non-genetic factors (e.g., smoking) can be highly variable, thus the focus of this investigation was to isolate the contribution conferred by genetic variation alone, in order to determine whether a more comprehensive collection of SNPs could further improve prediction accuracy. The methodology used in the clinical validation of the 13-SNP test panel was subsequently applied to two additional panels of markers which included variants that overlapped with markers within the 13-SNP panel. Both test panels were evaluated in the large collective cohort by using a validation step. Testing the two panels in a large collection of subjects from different centers assembled from several independent collections was designed to minimize the introduction of selection bias inherent in a single cohort study. Additionally, the use of an independent validation sample was intended to aggressively challenge the 13-SNP panel, to anticipate performance metrics in a broader clinical setting more accurately. Running the three test panels (three SNPs, six SNPs and 13 SNPs) on the same samples allowed for the comparison of performance metrics based exclusively on genetic variants.

### Methods

Subjects

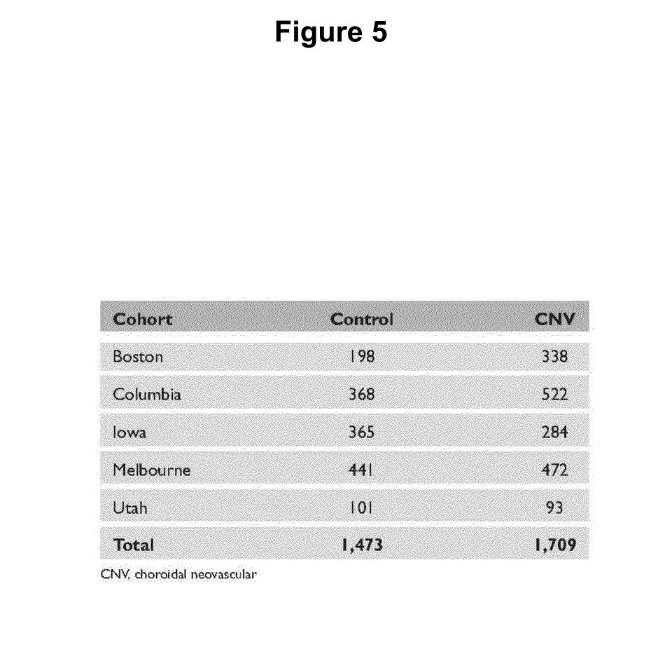

Four well-characterized cohorts (Iowa, Boston, Columbia, and Melbourne) and one recently acquired cohort (Utah), together comprised 1,709 patients diagnosed with CNV and 1,473 disease-free controls (for which genotyping data were already available), were assessed (FIG. 5). All individuals were of white European ancestry, 60 years of age and older and matched for age. All patients had given their consent and were enrolled under Institutional Review Board-approved protocols. The methods used in this study conformed to the tenets of the Declaration of Helsinki (2000) of the World Medical Association. Study subjects were examined and photographed by trained ophthalmologists; fundus photographs were graded according to published standardized classification systems. The worst affected eye of each case was used for classification purposes. All cohorts were case controlled, with the exception of the Boston sib-pair cohort. Index patients in the Boston cohort aged 60 years or older were included in the analyses and had CNV, (e.g., subretinal hemorrhage, fibrosis or fluorescein angiographic presence of neovascularization documented at the time of, or prior to, enrollment in the study) in at least one eye. The unaffected siblings had normal maculae at an age older than that at which the index patient was first diagnosed with CNV. The Utah case-control cohort was recently ascertained at the John A. Moran Eye Center, University of Utah, in Salt Lake City, Utah, in a fashion identical to that of the Iowa cohort.

Markers

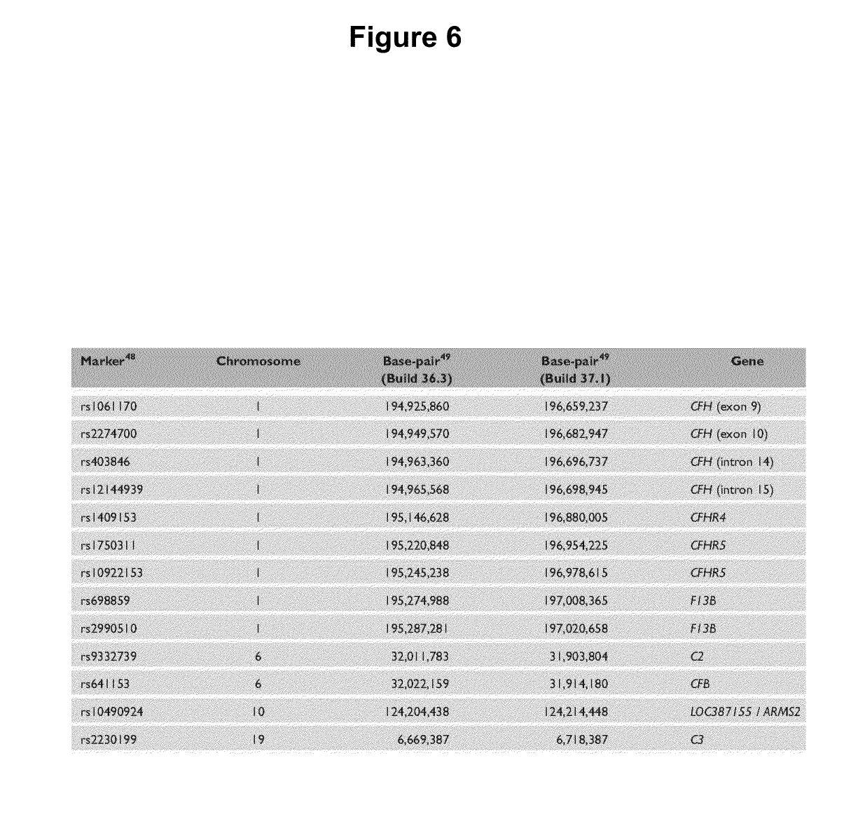

Thirteen SNPs, spanning four physically separate genomic loci, were genotyped in all five cohorts (FIG. 6). One locus spans the CFH, CFHR4, CFHR5 and F13B genes and includes nine SNPs; the second includes two SNPs, one each in C2 and CFB; the third includes a single SNP in C3; and the fourth includes a single SNP in ARMS2. One of the CFH SNPs (rs12144939) included in the panel tags the CFHR3/1 deletion. The 13 SNPs were selected on the basis of several characteristics including, for example, magnitude of estimated effect size and power to resolve clinically relevant haplotypes.

Statistical Methods

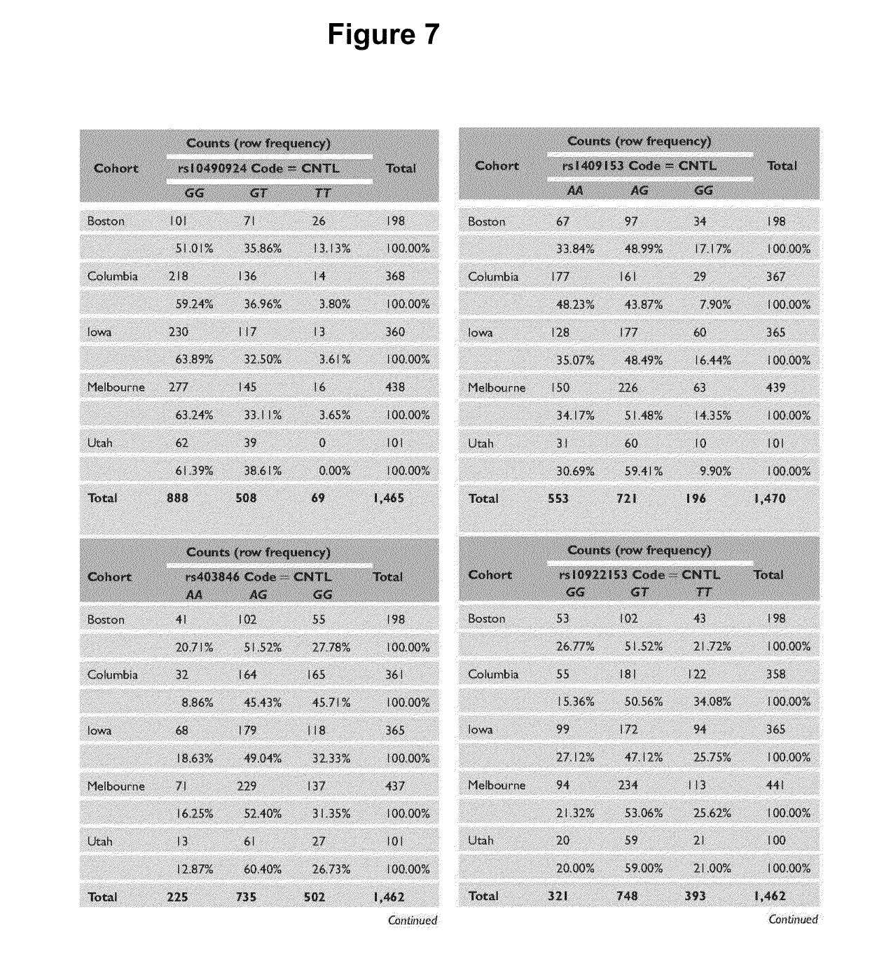

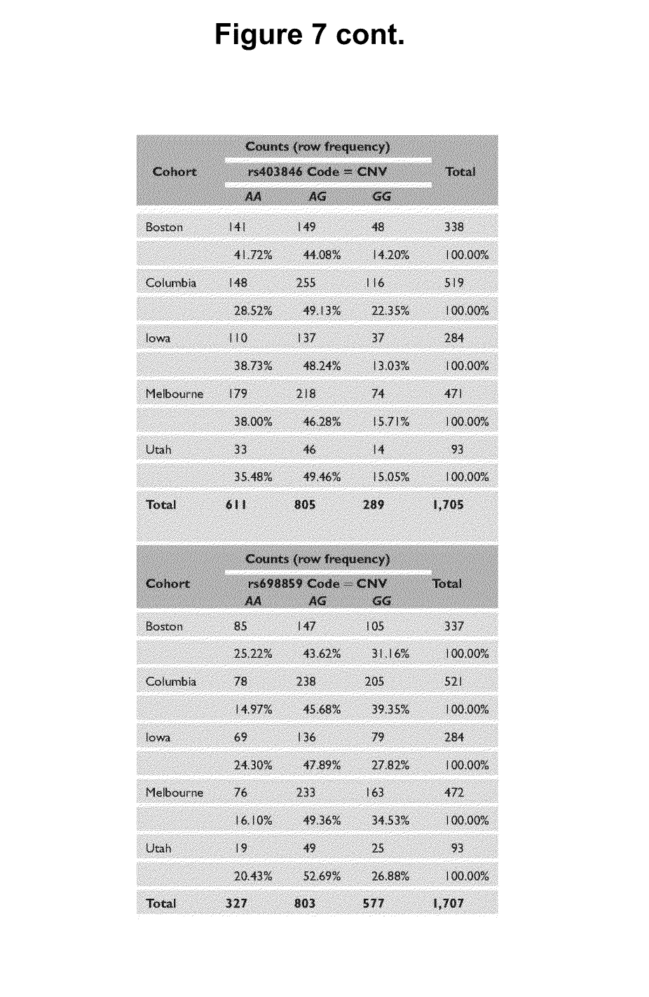

Previous analyses of each cohort involved standard quality checks and exclusions. Prior to analysis, the consistency of the assignment of the DNA strand used to detect the SNPs was assessed for all available datasets and any inconsistencies resolved. The percentage of missing data and the genotype frequencies were calculated and tabulated for each SNP, both by study and overall (FIG. 7). No SNPs showed significant deviation from Hardy-Weinberg equilibrium in the control population (P less than 0.05).

In order to determine the appropriateness of pooling the available cohorts, a chi-squared test of homogeneity of allele frequency was applied to compare frequencies across cohorts. Cohorts or subcohorts found to be a source of a departure from homogeneity of allele frequency (chi square P less than 0.001) were excluded from the main analysis.

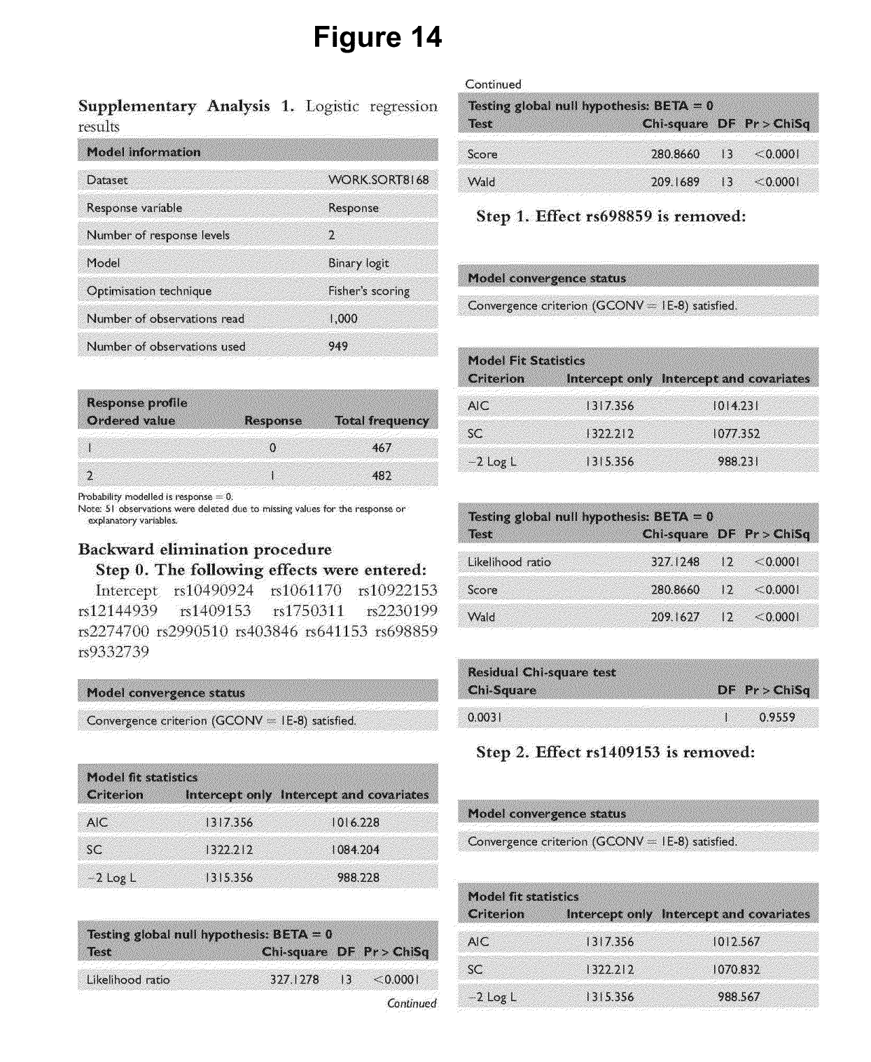

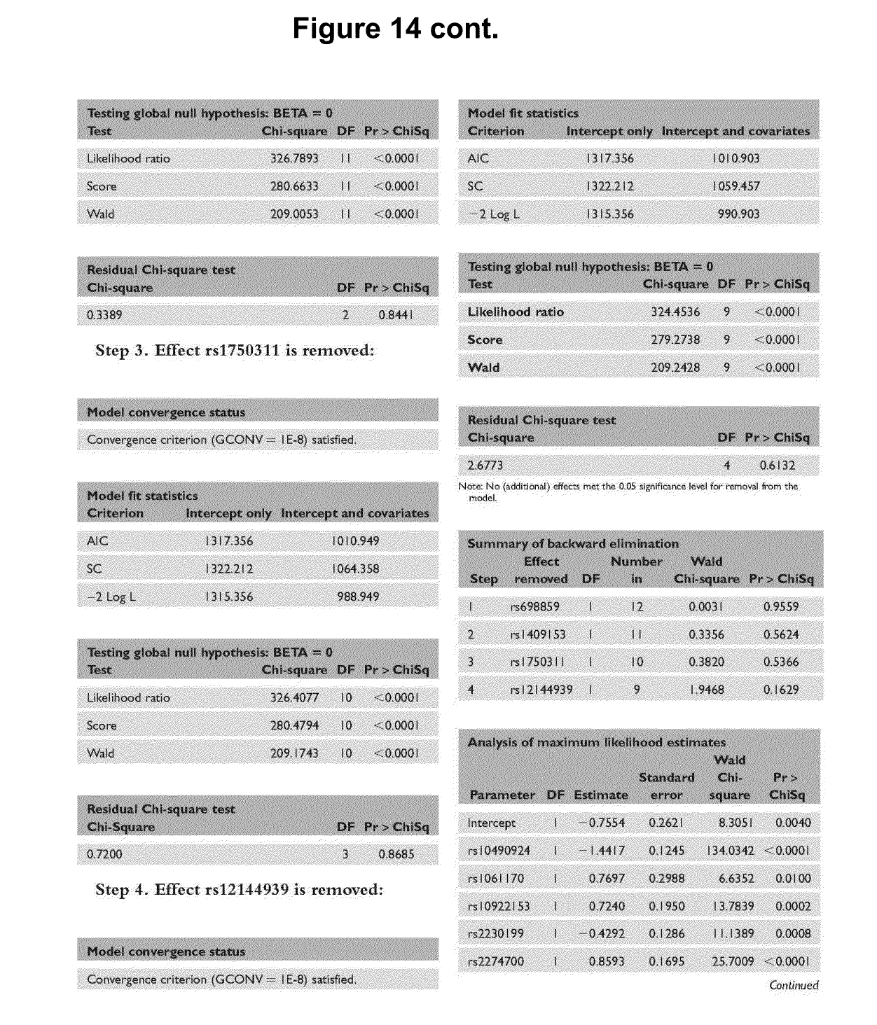

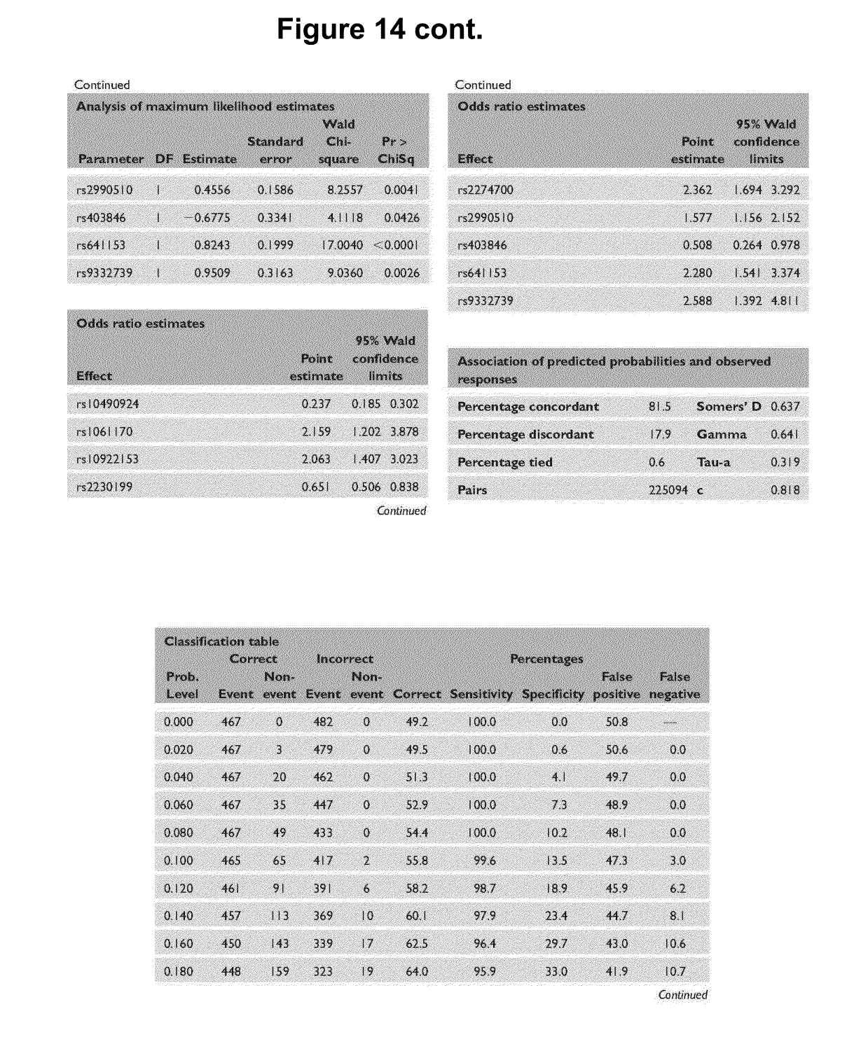

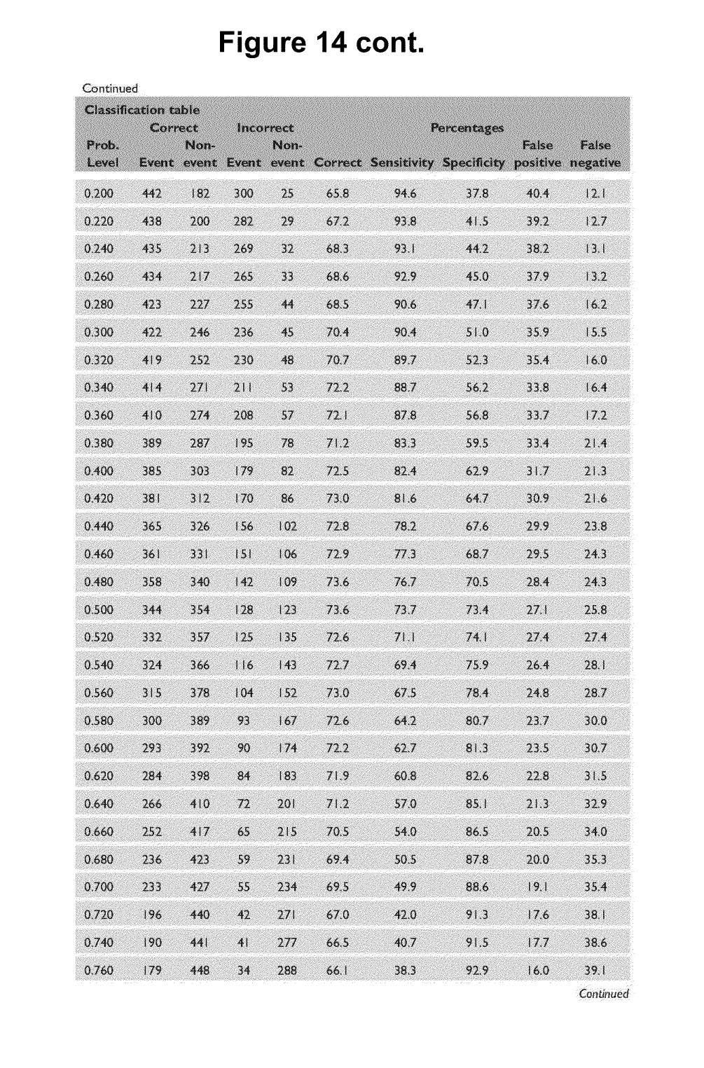

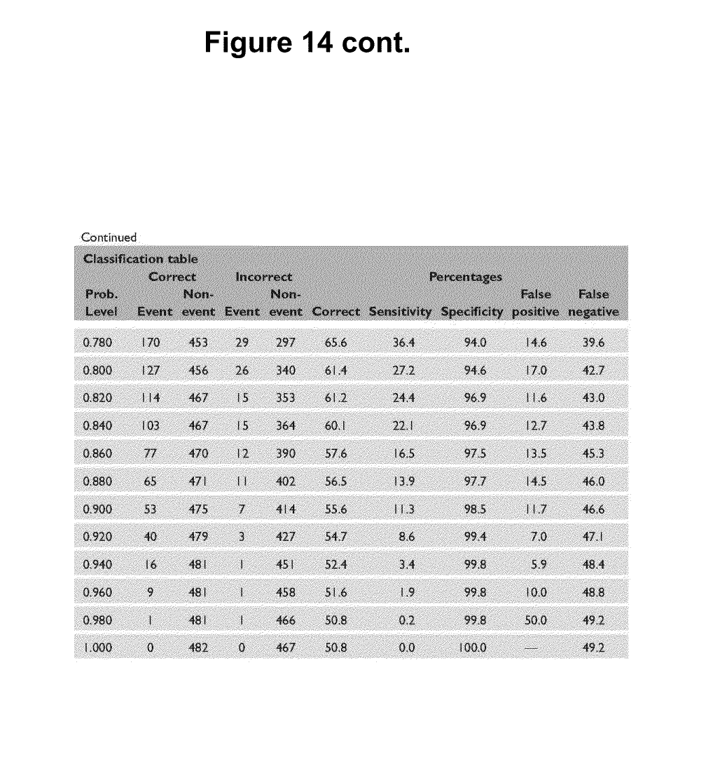

Individuals with CNV were compared with a control group of subjects with no recorded disease. Genotypic multivariate and univariate unconditional logistic regression analyses were performed to evaluate the relationships between risk of CNV and the additively coded genotypes (FIG. 14). Odds ratios (ORs) and 95% confidence intervals (CIs) were calculated. The full 13-SNP panel was evaluated both with and without demographic factors of age and sex. Backward elimination was performed on the training set using a threshold of P less than 0.05.

Two published test models containing, respectively, three and six SNPs, and a nine-SNP model generated from backward elimination, were compared with the 13-SNP panel in terms of AUC in training and independent validation. In the event that an SNP was not present in the 13-SNP panel, a SNP with demonstrated linkage disequilibrium was used as a surrogate.

Training of classifiers was performed using 500 cases and 500 controls balanced by age and sex and randomly selected from the whole cohort. The remaining 322 controls and 632 cases were used for validation. In both analyses, ten-fold cross validation was applied. The predicted probability of affliction for each subject was calculated by applying the inverse-logit function; sensitivity, specificity and AUC were derived to assess classification performance.

A risk score for CNV was calculated as follows:

Sj=intercept+Σ(i to n)βi*Xi Equation A

where Sj is the risk score for subject j, βi is the adjusted log-odds ratio for Xi, the additively coded genotype at marker i, and n is the total number of markers (e.g., 13 markers). The probability of risk for subject j was calculated as:

pj=exp(Sj)/[1+exp(Sj)] Equation B.

The optimal classification threshold was determined on the basis of accuracy, defined as the proportion of correct predictions observed in cases and controls. Different levels of prevalence, reflecting age-specific differences, were considered. The accuracy in the validation set was determined, and positive and negative predictive values were calculated. Calibration was assessed graphically as histograms showing disease incidence at different levels of predicted risk for controls and cases.

The area under the receiver operating characteristic (ROC) curve and CIs were estimated using SAS Macro % ROC. In addition, c-statistics and CIs were calculated for the training, tenfold cross validation and validation datasets. All analyses were conducted using SAS 9.1.

### Results

The average ages (+/−standard deviation [SD]) of cases and controls among all cohorts were 76.4 (+/−7.3) and 76.5 (+/−7.1) years, respectively, and the differences were not significant (p=0.86). Age matching was applied during cohort ascertainment. The chi-square test was used to assess homogeneity of allele frequency across cohorts. Frequencies of markers rs10490924, rs403846, rs1409153, rs698859, rs403846 and rs10922153 were significantly different (P less than 0.001) across cohorts. The frequencies of four markers—rs10490924, (ARMS2) rs403846, (CFH) rs1409153 (CFHR4) and rs10922153 (CFHR5)—in the control population and two markers—rs698859 (F13B) and rs403846 (CFH)—in the CNV population were unbalanced (FIG. 7). Removal of the Columbia University cohort eliminated four of the five deviations, leaving only one SNP (rs10490924) outstanding in the Boston control population. The Boston controls and Columbia cases and controls were excluded from the main analyses based on these observations. The remaining study population contained 1,132 CNV cases and 822 controls. For the purposes of the current analysis, investigations into the differences were not pursued but could be evaluated in the future by performing structure analysis to identify potential causes for the observed differences.

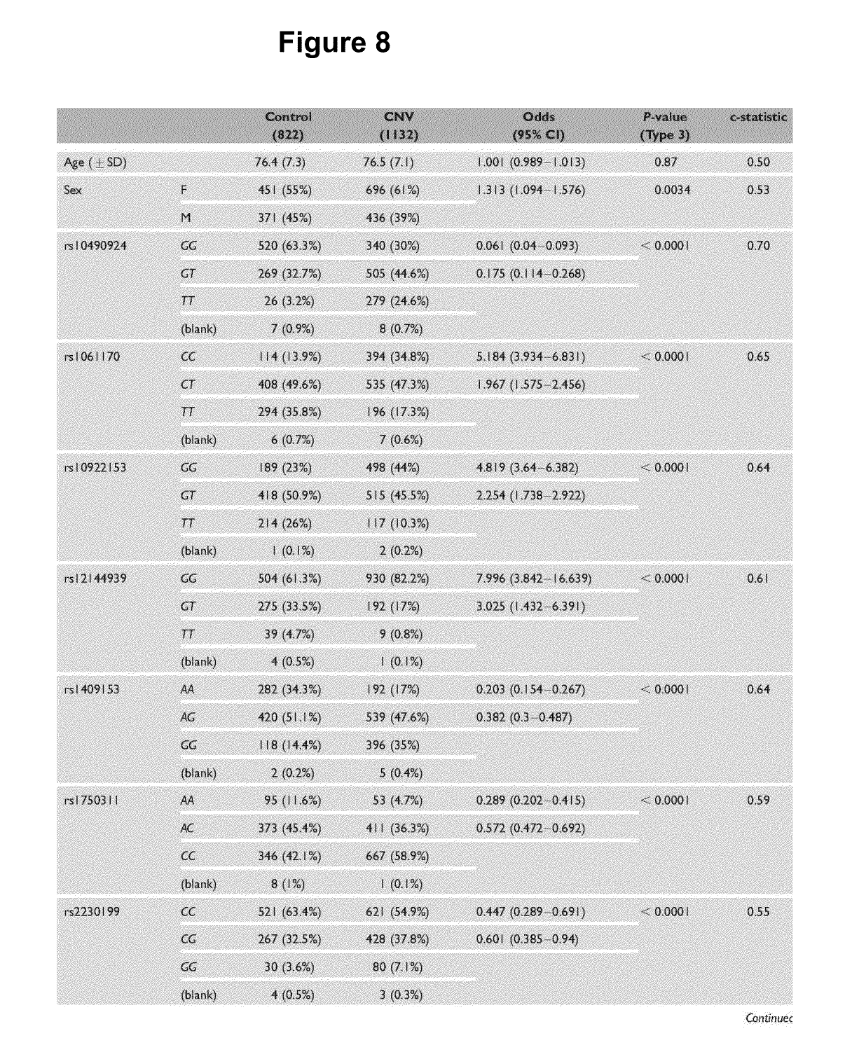

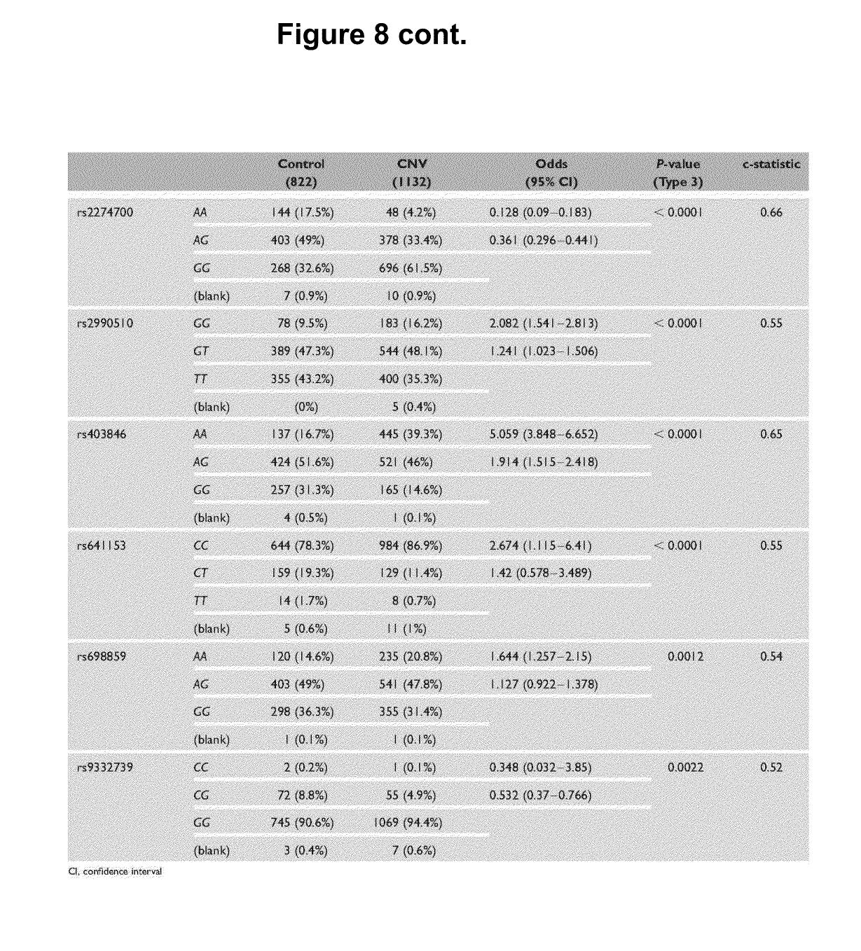

FIG. 8 shows unadjusted association test results between the demographic and genetic factors and the risk of CNV. All factors except age were associated with risk of CNV. The c-statistic column shows the ability of a genetic factor to predict CNV risk. SNPs rs10490924, rs1061170, rs403846 and rs2274700 had c-statistics greater than or equal to 0.65.

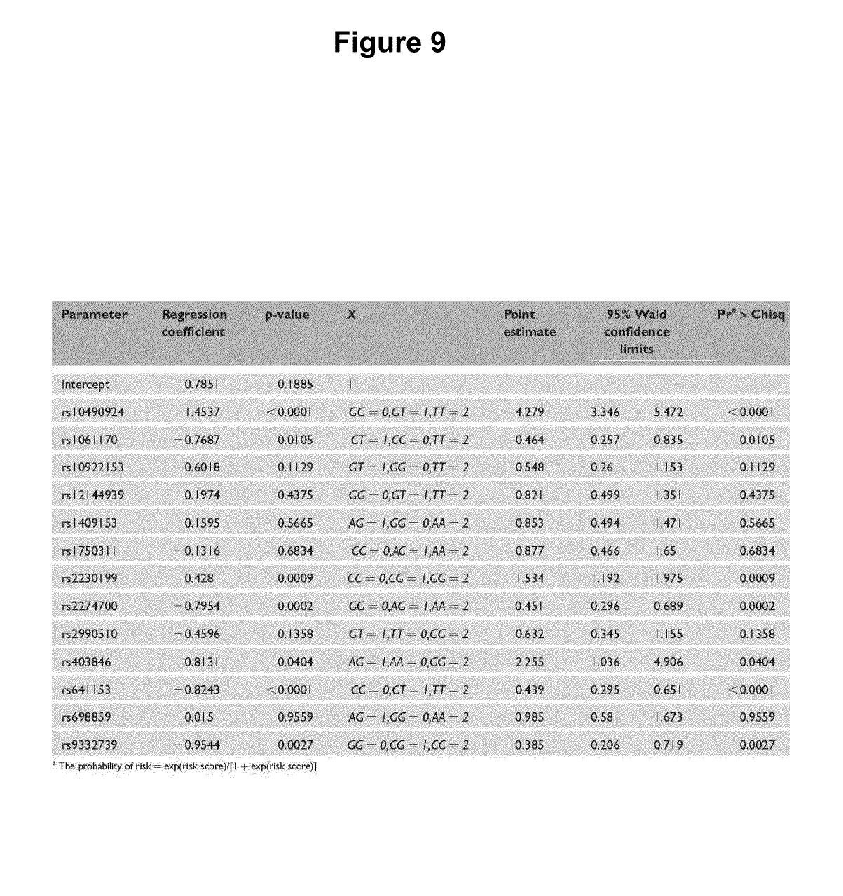

FIG. 9 shows multivariate adjusted ORs that were significantly associated with the risk of CNV, using the additive genotype model applied to the 13-SNP panel. The ARMS2 variant rs10490924 was positively associated with risk of CNV (OR 4.279, 95% CI 3.346-5.472, p less than 0.0001).

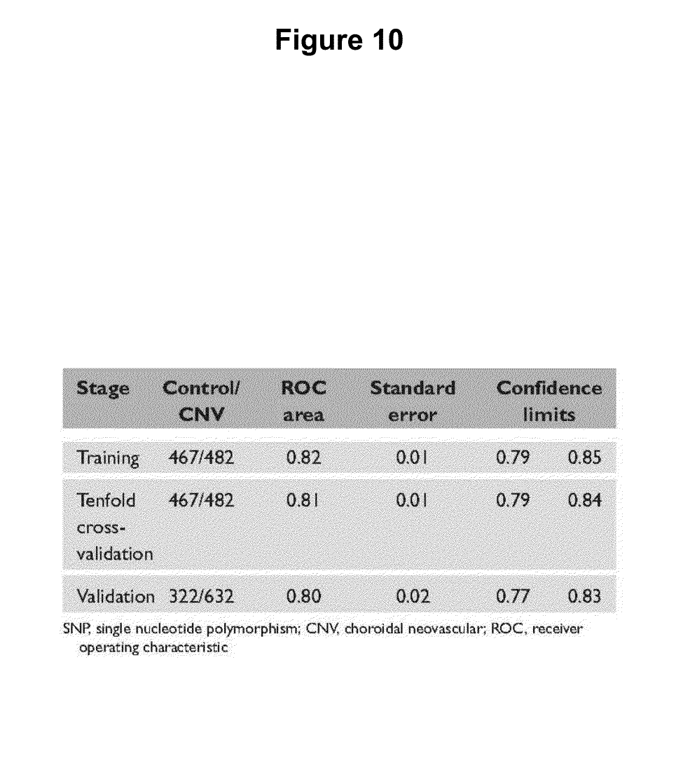

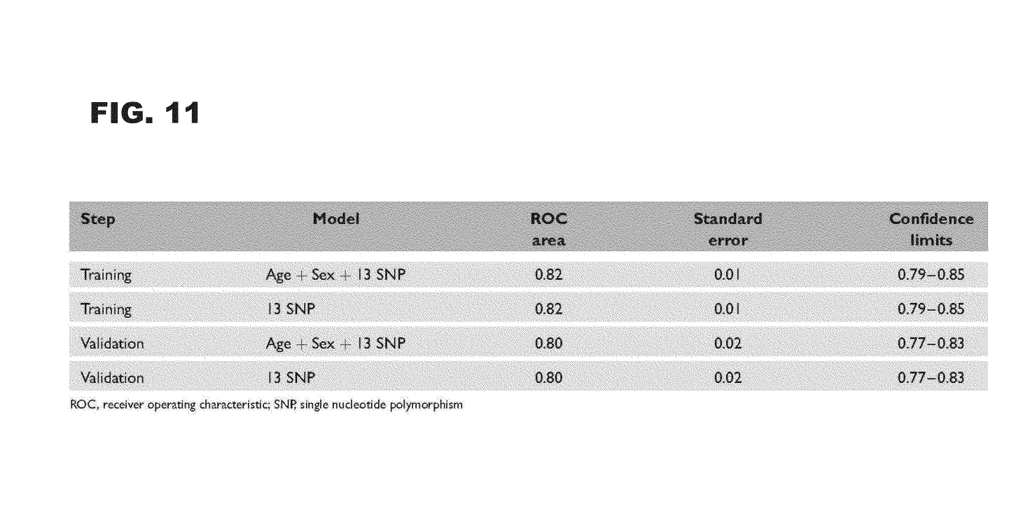

The performance of the 13-SNP panel to predict CNV relative to the control population was evaluated using tenfold cross-validation and an independent dataset. Independent datasets were scored using model parameters displayed in FIG. 9. FIG. 10 shows the AUC evaluated for training (AUC 0.82 [0.79-0.85]), tenfold cross-validation (AUC 0.81 [0.79-0.84]) and validation (AUC 0.79 [0.77-0.83]). The c-statistics results were identical to AUC. These data show that the difference in performance of the training and validation sets was not significant (P less than 0.05). There were no significant differences between the AUC curves for the training and validation datasets with demographic factors (age, sex) added into the test model (FIG. 11).

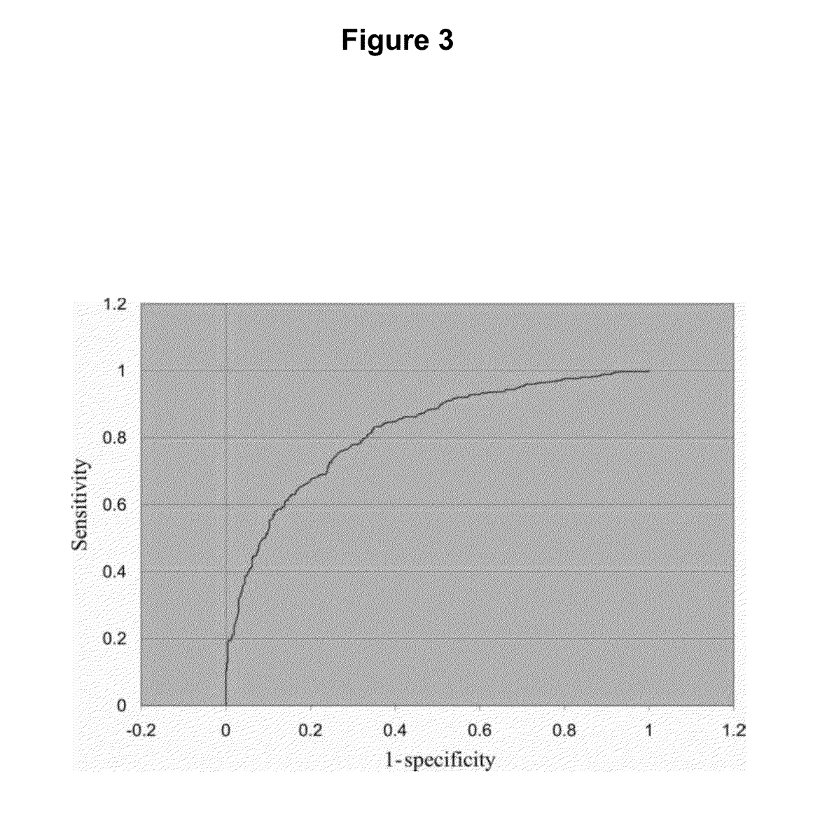

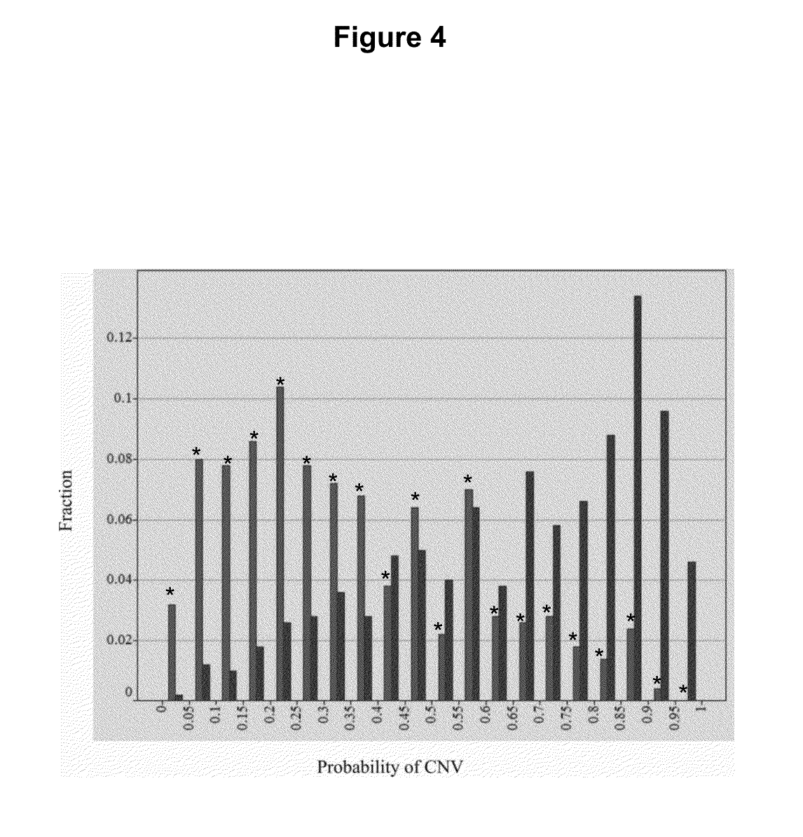

The sensitivity and specificity of predictions were calculated in an independent dataset using the test panels in FIG. 9. The ROC curve is shown in FIG. 3. The probability of the risk of CNV was plotted as histograms for controls and cases in the independent dataset in FIG. 4. Good separation was observed between the two groups, with cases from the dataset having a substantially higher probability of CNV versus controls, although some overlap was present.

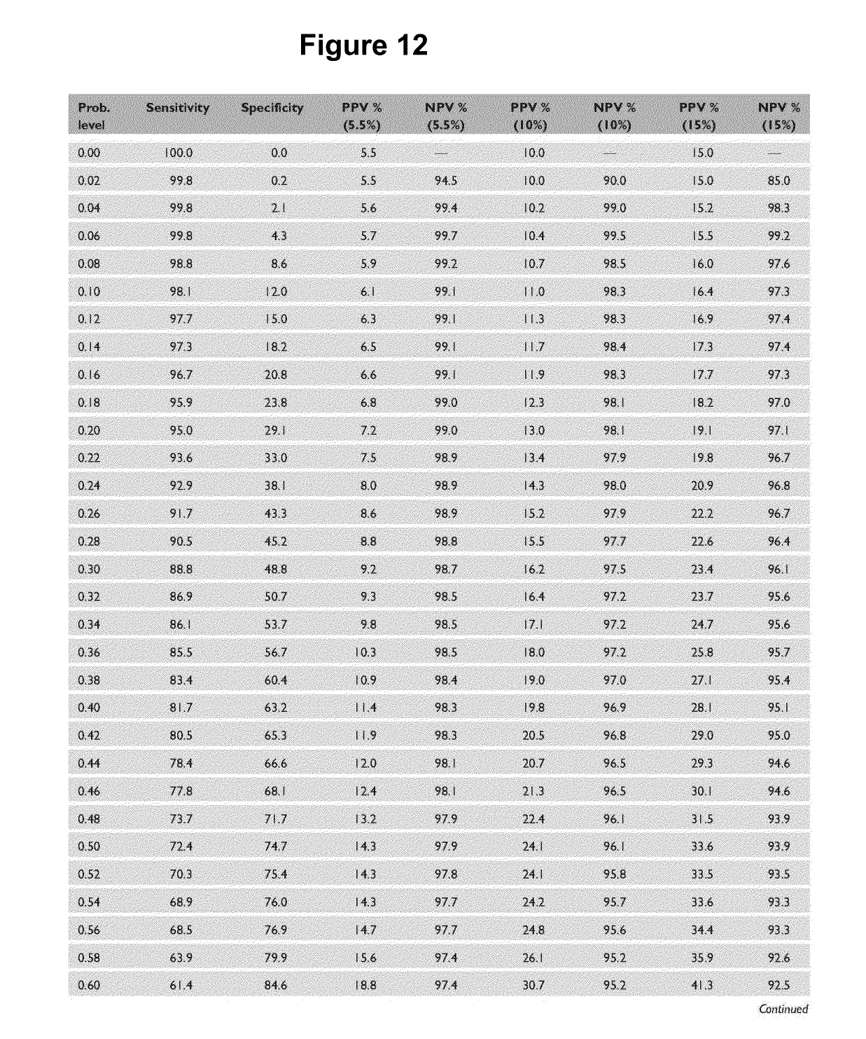

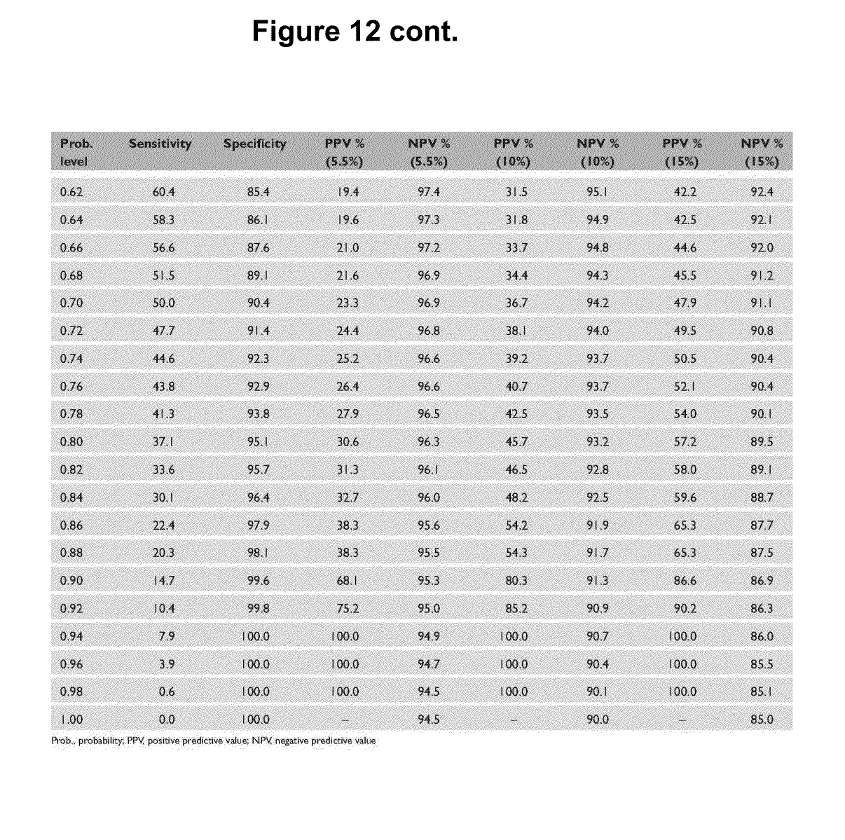

Accuracy, specificity, sensitivity, PPV and negative predicted values (NPV) are shown in FIG. 12 as a function of probability cut-off and three prevalence values. A cut-off of 0.4 corresponded to the highest accuracy (0.73), with a sensitivity of 0.82 and a specificity of 0.63. The PPV for 5.5%, 10% and 15% prevalence values were 0.11, 0.20 and 0.28, respectively. The NPVs were all above 0.95.

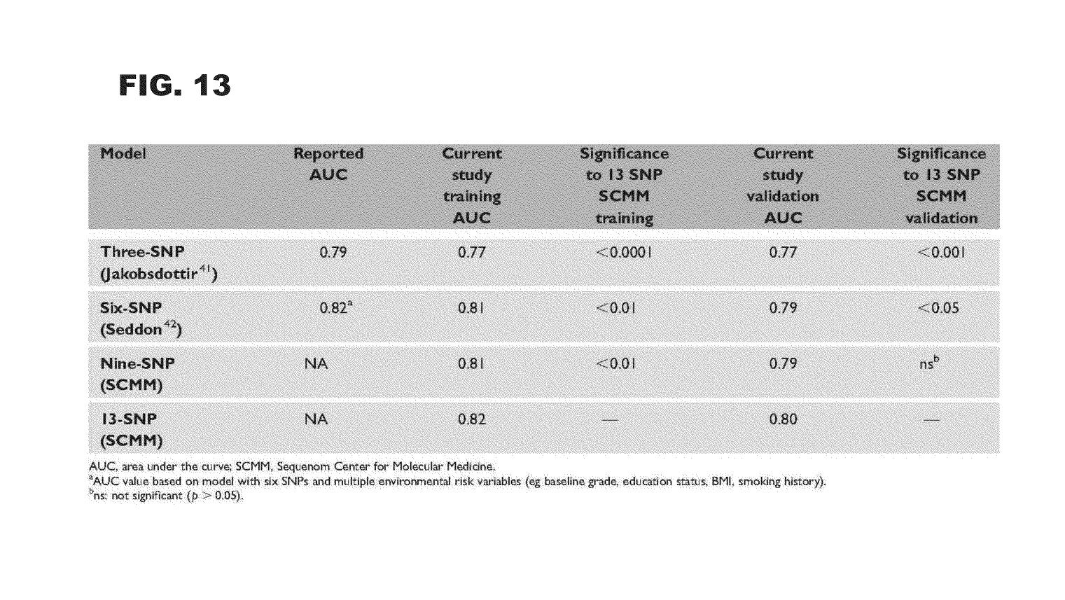

The 13-SNP panel was compared to other predictive models (FIG. 13). The differences in test performance were evaluated at training and validation stages. The performance of the 13-SNP panel was slightly better than that of the next best test. Results from a nine-SNP panel generated from the backwards elimination procedure realized gains in genotyping efficiency, with four fewer variants in the panel, while demonstrating only slightly lower performance in terms of AUC.

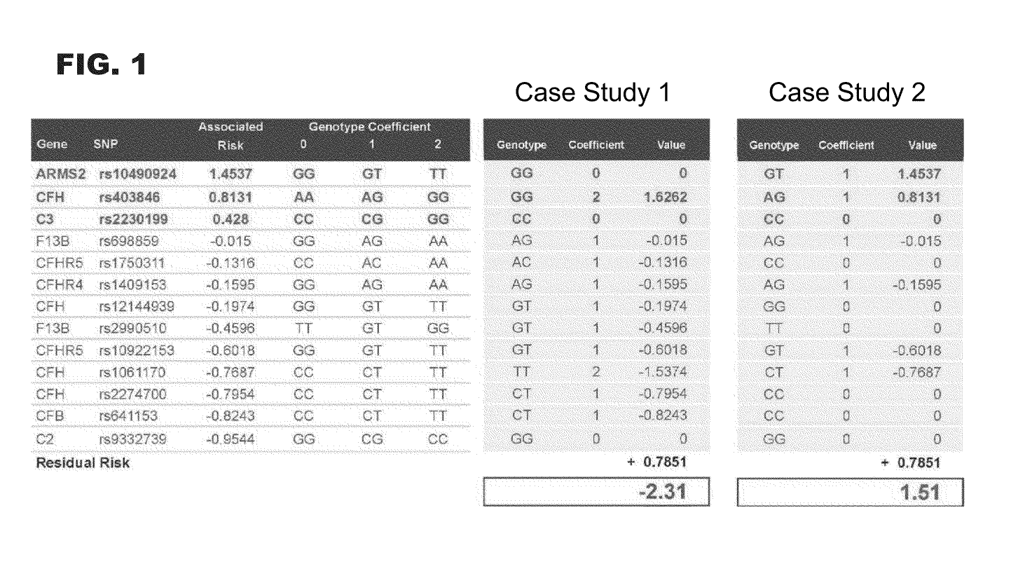

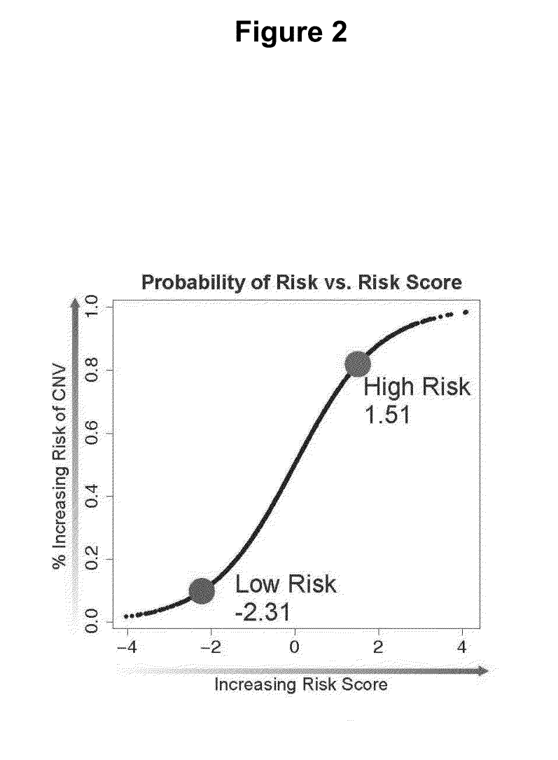

Patient probability of developing CNV was determined for two individual case studies (FIG. 1). A risk score was calculated by 1) multiplying an individual's genotype coefficient (assigned a value of 0, 1 or 2) by a value associated with the risk of each marker; 2) adding the products from step 1 together; and 3) adding a residual risk value. Residual risk (constant value of 0.7851) is a component of risk of developing CNV by factors not accounted for in the above model. For case study 1, a risk score of −2.31 was calculated; and for case study 2, a risk score of 1.51 was calculated. For case study 1, patient probability of developing CNV was 9% at age 60 or older (low risk). For case study 2, patient probability of developing CNV was 82% at age 60 or older (high risk). An example plot of probability of risk versus risk score is presented in FIG. 2.

### Discussion

The 13-SNP panel had a clinical sensitivity of 82 percent and a specificity of 63 percent, achieving clinical performance metrics comparable with models with fewer SNPs that include self-reported and/or non-static risk factors. The PPV of the panel was evaluated at different levels of prevalence, reflecting ranges covering estimates of late-stage disease in individuals older than 40, older than 65, and older than 80 years of age in the general population. More favorable estimates of PPV were observed as the prevalence of disease increased with age. The values obtained revealed 11% PPV at 5.5% prevalence, 20% PPV at 10% prevalence and 28% PPV at 15% prevalence in the general population. The prevalence figures reflect conservative estimates of late-stage disease in the general population. PPVs improved significantly when applied to the population of patients diagnosed with early stages of disease.

### Example 2

### Genetic Variants in Complement Factor H, Complement Factor H Related 5, ARMS2 and Factor B that Show Association with Response to Anti-VEGF Therapy

In this example, genetic variants were identified that stratify major phenotypic subtypes of choroidal neovascular (CNV) age related macular degeneration (AMD) and/or influence response to anti-VEGF therapy. An association was evaluated between genotype and phenotype assignment corresponding to the major and minor sub types of CNV to determine the association with response to anti-VEGF therapy. Additionally, an assessment of risk score, which also can be used as a quantitative measure for estimating the risk of developing CNV, was tested as a proxy to estimate if genetic burden aligned with more aggressive phenotypes and/or less responsive treatment categories.

### Methods

Each consented genetic specimen was tested across a panel of SNPs associated with CNV. 327 study subjects were classified according to major and minor phenotypic subtypes of CNV and categorized according to their response to anti-VEGF therapy. The following pairs of subtype groups were considered:
1. Classic vs. occult
2. Classic vs. RAP
3. Occult vs. RAP
4. Polyps vs. remainder
5. Arteriolarization vs. remainder
6. Retinal pigment epithelial detachment (RPED) vs. remainder
7. Peripapillary neovascularization (PPP) vs. remainder
8. Anti-VEGF sensitive vs. remainder
9. Anti-VEGF dependent vs. remainder
10. Anti-VEGF resistant vs. remainder

In some instances, the following pair also was considered:
11. Bilateral vs. Unilateral

Patients were subject to an initial induction treatment of 0.5 mg Ranibizumab. The endpoint of the induction was elimination of leakage and achievement of neovascular resolution. The maintenance endpoint was maintenance of a stable state. Categorical assignment of drug response was based on the achievement of successful induction and maintenance endpoints.

Genetic markers were individually tested for association under an additive genetic model.

Specifically, thirteen genetic markers (rs1061170, rs2274700, rs403846, rs12144939, rs1409153, rs1750311, rs10922153, rs698859, rs2990510, rs9332739, rs641153, rs10490924 and rs2230199) and four CFH haplotypes were individually tested for association under an additive genetic model, for the categorizations above. Association testing was conducted both with and without adjustment for smoking (never/past/current). A mean risk score was calculated for the groups above and a t-test was performed to compare the means for each pair. Means stratified by smoking status also were calculated. A custom R-script was written to perform the analyses.

### Results

The groups assigned for the 327 samples included 89 subjects with classic CNV, 172 with occult CNV, 15 with RAP and 270 subjects with bilateral disease. Eight genotypes fell outside of the allele spectrum for the marker and were excluded (Table 1). Two heterozygous genotypes (GA for rs14909153 and CA for rs1750311) were retained despite non-conventional allele-ordering.

TABLE 1
Genotypes excluded from analysis

| Marker | Change | Sequenom ID | Genotype |
|---|---|---|---|
| rs2274700 | C/T | 1204000021 | CG |
| rs1409153 | A/G | 1200900095 | CC |
| rs1750311 | A/C | 1200900095 | GG |
| rs10922153 | G/T | 1200900095 | AG |
| rs698859 | A/G | 1200900095 | GT |
| rs9332739 | C/G | 1202700104 | GCG |
| rs9332739 | C/G | 1201900160 | GT |
| rs641153 | C/T | 1201900160 | CG |

Minor allele frequency (MAF) estimates for 13 markers were measured and are provided in Table 2. One marker, rs9332739, showed a minor allele frequency less than 0.05. Levels of missing data were low (Table 2).

TABLE 2
Minor allele frequency and percent
missing genotypes for 13 markers

| Marker | MAF | % Missing |
|---|---|---|
| rs1061170 | 0.4388 | 0.0000 |
| rs2274700 | 0.2331 | 0.0031 |
| rs403846 | 0.4205 | 0.0000 |
| rs12144939 | 0.1101 | 0.0000 |
| rs1409153 | 0.4617 | 0.0031 |
| rs1750311 | 0.2776 | 0.0031 |
| rs10922153 | 0.3850 | 0.0031 |
| rs698859 | 0.4218 | 0.0031 |
| rs2990510 | 0.4052 | 0.0000 |
| rs10490924 | 0.3746 | 0.0000 |
| rs2230199 | 0.2446 | 0.0000 |
| rs9332739 | 0.0247 | 0.0092 |
| rs641153 | 0.0567 | 0.0031 |

A total of 340 non-independent association tests were conducted evaluating 10 phenotype-pairs, thirteen markers plus 4 haplotypes with and without adjustment for smoking. Some of the association tests are presented in Table 3A and Table 3B below.

TABLE 3A

| Group 1 | Group 2 | Marker | N1 | N2 | OR | 95CI_L | 95CI_U |
|---|---|---|---|---|---|---|---|
| RPED | Remainder | rs10490924 | 31 | 244 | 1.81 | 1.09 | 3.03 |
| Polyps | Remainder | rs641153 | 115 | 160 | 2.34 | 1.1 | 4.95 |
| Anti_VEGF_Sensitive | Remainder | rs10922153 | 88 | 187 | 0.65 | 0.44 | 0.96 |
| Anti_VEGF_Dependent | Remainder | rs403846 | 58 | 217 | 0.63 | 0.41 | 0.97 |
| Anti_VEGF_Dependent | Remainder | rs1061170 | 58 | 217 | 0.65 | 0.42 | 1 |
| PPP | Remainder | rs10490924 | 29 | 246 | 0.57 | 0.32 | 1.01 |
| Classic | Occult | rs2990510 | 89 | 171 | 0.69 | 0.46 | 1.03 |
| Anti_VEGF_Dependent | Remainder | rs2990510 | 58 | 217 | 1.51 | 0.97 | 2.35 |
| Anti_VEGF_Sensitive | Anti_VEGF_Dependent | rs2274700 | 88 | 58 | 1.76 | 0.94 | 3.32 |
| PPP | Remainder | rs10922153 | 29 | 246 | 1.61 | 0.92 | 2.83 |
| Anti_VEGF_Resistant | Remainder | rs12144939 | 10 | 265 | 2.54 | 0.83 | 7.81 |
| RPED | Remainder | H1 | 31 | 244 | 3.37 | 0.78 | 14.62 |
| Anti_VEGF_Dependent | Remainder | rs2274700 | 58 | 217 | 0.64 | 0.37 | 1.1 |
| Anti_VEGF_Dependent | Remainder | rs12144939 | 58 | 217 | 0.49 | 0.2 | 1.18 |
| PPP | Remainder | H3 | 29 | 246 | 0.47 | 0.18 | 1.19 |
| Anti_VEGF_Sensitive | Anti_VEGF_Dependent | rs403846 | 88 | 58 | 1.49 | 0.9 | 2.46 |
| Arteriolarization | Remainder | rs2230199 | 77 | 198 | 0.7 | 0.44 | 1.1 |
| Polyps | Remainder | rs9332739 | 115 | 160 | 0.3 | 0.06 | 1.4 |
| Anti_VEGF_Sensitive | Remainder | rs1750311 | 88 | 187 | 0.73 | 0.48 | 1.1 |
| Anti_VEGF_Dependent | Remainder | H4 | 58 | 217 | 0.5 | 0.2 | 1.23 |
| PPP | Remainder | H1 | 29 | 246 | 0.51 | 0.21 | 1.23 |
| Anti_VEGF_Dependent | Remainder | rs10490924 | 58 | 217 | 1.34 | 0.9 | 1.98 |
| Polyps | Remainder | H2 | 115 | 160 | 0.65 | 0.36 | 1.17 |
| Anti_VEGF_Sensitive | Anti_VEGF_Dependent | H4 | 88 | 58 | 2.08 | 0.77 | 5.62 |
| Anti_VEGF_Sensitive | Anti_VEGF_Dependent | rs12144939 | 88 | 58 | 2.08 | 0.77 | 5.62 |
| Classic | RAP | H3 | 89 | 15 | 0.45 | 0.15 | 1.36 |
| Anti_VEGF_Sensitive | Remainder | rs10490924 | 88 | 187 | 1.28 | 0.91 | 1.81 |
| Classic | Occult | rs2230199 | 89 | 171 | 0.73 | 0.47 | 1.13 |
| Classic | Occult | rs9332739 | 89 | 171 | 2.37 | 0.7 | 8 |
| Anti_VEGF_Sensitive | Anti_VEGF_Dependent | rs1061170 | 88 | 58 | 1.41 | 0.86 | 2.29 |
| PPP | Remainder | rs1750311 | 29 | 246 | 1.49 | 0.84 | 2.62 |
| Occult | RAP | rs10490924 | 171 | 15 | 1.68 | 0.79 | 3.57 |
| Anti_VEGF_Dependent | Remainder | rs10922153 | 58 | 217 | 0.74 | 0.47 | 1.15 |
| Classic | Occult | H3 | 89 | 171 | 0.69 | 0.39 | 1.2 |
| Anti_VEGF_Resistant | Remainder | rs698859 | 10 | 265 | 0.53 | 0.2 | 1.4 |
| Anti_VEGF_Sensitive | Remainder | H2 | 88 | 187 | 1.47 | 0.82 | 2.65 |
| Anti_VEGF_Dependent | Remainder | rs1750311 | 58 | 217 | 0.73 | 0.45 | 1.18 |
| Arteriolarization | Remainder | rs9332739 | 77 | 198 | 2.2 | 0.65 | 7.43 |
| RPED | Remainder | rs10922153 | 31 | 244 | 0.7 | 0.39 | 1.24 |
| Anti_VEGF_Dependent | Remainder | rs1409153 | 58 | 217 | 0.77 | 0.51 | 1.17 |
| PPP | Remainder | rs403846 | 29 | 246 | 1.4 | 0.82 | 2.39 |
| Classic | Occult | rs698859 | 89 | 171 | 1.25 | 0.87 | 1.8 |
| Polyps | Remainder | rs12144939 | 115 | 160 | 1.42 | 0.8 | 2.53 |
| Classic | Occult | H2 | 89 | 171 | 1.43 | 0.79 | 2.61 |
| Arteriolarization | Remainder | H2 | 77 | 198 | 1.44 | 0.78 | 2.64 |
| Anti_VEGF_Dependent | Remainder | rs698859 | 58 | 217 | 0.78 | 0.51 | 1.19 |
| RPED | Remainder | rs2990510 | 31 | 244 | 1.39 | 0.79 | 2.45 |
| Anti_VEGF_Sensitive | Anti_VEGF_Dependent | H2 | 88 | 58 | 1.6 | 0.72 | 3.59 |
| Anti_VEGF_Dependent | Remainder | rs641153 | 58 | 217 | 0.54 | 0.18 | 1.57 |
| Classic | RAP | rs2230199 | 89 | 15 | 0.59 | 0.23 | 1.51 |
| Anti_VEGF_Sensitive | Remainder | H3 | 88 | 187 | 0.73 | 0.42 | 1.27 |
| Polyps | Remainder | H4 | 115 | 160 | 1.42 | 0.75 | 2.66 |
| Anti_VEGF_Resistant | Remainder | H4 | 10 | 265 | 2.15 | 0.54 | 8.65 |
| Anti_VEGF_Sensitive | Remainder | rs2274700 | 88 | 187 | 1.26 | 0.83 | 1.92 |
| Arteriolarization | Remainder | rs10922153 | 77 | 198 | 1.23 | 0.84 | 1.81 |
| RPED | Remainder | rs2230199 | 31 | 244 | 1.38 | 0.76 | 2.5 |
| Classic | RAP | rs10490924 | 89 | 15 | 1.56 | 0.67 | 3.65 |
| PPP | Remainder | rs2990510 | 29 | 246 | 0.73 | 0.4 | 1.34 |
| RPED | Remainder | rs2274700 | 31 | 244 | 0.7 | 0.35 | 1.41 |
| Anti_VEGF_Sensitive | Anti_VEGF_Dependent | rs2990510 | 88 | 58 | 0.77 | 0.46 | 1.29 |
| Arteriolarization | Remainder | rs12144939 | 77 | 198 | 0.71 | 0.36 | 1.42 |
| RPED | Remainder | rs1409153 | 31 | 244 | 0.77 | 0.45 | 1.32 |
| Anti_VEGF_Resistant | Remainder | H3 | 10 | 265 | 0.47 | 0.1 | 2.26 |
| Anti_VEGF_Resistant | Remainder | rs9332739 | 10 | 265 | 2.81 | 0.32 | 24.39 |
| Occult | RAP | H1 | 171 | 15 | 1.78 | 0.53 | 5.98 |
| Anti_VEGF_Resistant | Remainder | H2 | 10 | 265 | 0.37 | 0.05 | 2.99 |
| PPP | Remainder | rs2274700 | 29 | 246 | 1.34 | 0.72 | 2.47 |
| RPED | Remainder | rs1061170 | 31 | 244 | 0.78 | 0.45 | 1.34 |
| RPED | Remainder | rs1750311 | 31 | 244 | 0.75 | 0.4 | 1.4 |
| RPED | Remainder | H2 | 31 | 244 | 0.63 | 0.23 | 1.72 |
| Classic | RAP | H1 | 89 | 15 | 1.79 | 0.5 | 6.4 |
| Classic | RAP | rs403846 | 89 | 15 | 0.71 | 0.34 | 1.5 |
| Anti_VEGF_Sensitive | Anti_VEGF_Dependent | rs641153 | 88 | 58 | 1.73 | 0.52 | 5.81 |
| Classic | Occult | rs10490924 | 89 | 171 | 0.85 | 0.6 | 1.21 |
| Arteriolarization | Remainder | H1 | 77 | 198 | 1.38 | 0.66 | 2.87 |
| Anti_VEGF_Sensitive | Anti_VEGF_Dependent | rs698859 | 88 | 58 | 1.24 | 0.75 | 2.06 |
| Arteriolarization | Remainder | rs1750311 | 77 | 198 | 1.19 | 0.8 | 1.78 |
| Arteriolarization | Remainder | rs2990510 | 77 | 198 | 0.84 | 0.56 | 1.26 |
| RPED | Remainder | rs403846 | 31 | 244 | 0.79 | 0.46 | 1.36 |
| Occult | RAP | rs698859 | 171 | 15 | 0.73 | 0.34 | 1.54 |
| Anti_VEGF_Dependent | Remainder | rs2230199 | 58 | 217 | 0.81 | 0.49 | 1.34 |
| PPP | Remainder | rs12144939 | 29 | 246 | 1.42 | 0.61 | 3.27 |
| Occult | RAP | rs403846 | 171 | 15 | 0.74 | 0.35 | 1.54 |
| Anti_VEGF_Sensitive | Remainder | H1 | 88 | 187 | 1.33 | 0.66 | 2.66 |
| Occult | RAP | rs1409153 | 171 | 15 | 0.74 | 0.35 | 1.55 |
| Occult | RAP | H3 | 171 | 15 | 0.65 | 0.22 | 1.88 |
| Polyps | Remainder | rs2230199 | 115 | 160 | 1.17 | 0.79 | 1.75 |
| Polyps | Remainder | rs2990510 | 115 | 160 | 1.15 | 0.8 | 1.66 |
| Arteriolarization | Remainder | H4 | 77 | 198 | 0.75 | 0.36 | 1.56 |
| PPP | Remainder | rs2230199 | 29 | 246 | 1.27 | 0.69 | 2.37 |
| Classic | Occult | rs2274700 | 89 | 171 | 1.18 | 0.77 | 1.81 |
| Anti_VEGF_Dependent | Remainder | H2 | 58 | 217 | 0.76 | 0.37 | 1.58 |
| Occult | RAP | rs2990510 | 171 | 15 | 1.34 | 0.6 | 2.99 |
| Classic | RAP | rs1409153 | 89 | 15 | 0.77 | 0.37 | 1.6 |
| Arteriolarization | Remainder | rs641153 | 77 | 198 | 0.74 | 0.31 | 1.74 |
| Anti_VEGF_Sensitive | Remainder | H4 | 88 | 187 | 1.25 | 0.65 | 2.42 |
| Arteriolarization | Remainder | H3 | 77 | 198 | 0.83 | 0.47 | 1.45 |
| Occult | RAP | rs1061170 | 171 | 15 | 0.79 | 0.38 | 1.65 |
| Anti_VEGF_Sensitive | Remainder | rs1409153 | 88 | 187 | 0.89 | 0.62 | 1.27 |
| Classic | RAP | rs1061170 | 89 | 15 | 0.79 | 0.37 | 1.68 |
| Polyps | Remainder | rs10922153 | 115 | 160 | 0.9 | 0.63 | 1.28 |
| Anti_VEGF_Sensitive | Remainder | rs2990510 | 88 | 187 | 1.12 | 0.76 | 1.65 |
| Anti_VEGF_Dependent | Remainder | H3 | 58 | 217 | 0.83 | 0.45 | 1.56 |
| Classic | RAP | H2 | 89 | 15 | 1.48 | 0.38 | 5.69 |
| Anti_VEGF_Resistant | Remainder | rs2230199 | 10 | 265 | 0.72 | 0.23 | 2.25 |
| RPED | Remainder | H3 | 31 | 244 | 1.25 | 0.58 | 2.69 |
| Arteriolarization | Remainder | rs403846 | 77 | 198 | 0.9 | 0.62 | 1.31 |
| Polyps | Remainder | rs1750311 | 115 | 160 | 1.11 | 0.77 | 1.61 |
| PPP | Remainder | H4 | 29 | 246 | 1.3 | 0.5 | 3.4 |
| Anti_VEGF_Resistant | Remainder | rs1061170 | 10 | 265 | 0.78 | 0.31 | 1.95 |
| Anti_VEGF_Sensitive | Remainder | rs2230199 | 88 | 187 | 0.89 | 0.58 | 1.36 |
| Anti_VEGF_Sensitive | Anti_VEGF_Dependent | rs1409153 | 88 | 58 | 1.13 | 0.71 | 1.8 |
| Occult | RAP | rs12144939 | 171 | 15 | 1.4 | 0.34 | 5.87 |
| Anti_VEGF_Resistant | Remainder | rs2990510 | 10 | 265 | 1.25 | 0.48 | 3.22 |
| Classic | RAP | H4 | 89 | 15 | 1.42 | 0.29 | 6.94 |
| Classic | RAP | rs12144939 | 89 | 15 | 1.42 | 0.29 | 6.94 |
| Anti_VEGF_Dependent | Remainder | H1 | 58 | 217 | 1.19 | 0.54 | 2.63 |
| PPP | Remainder | rs641153 | 29 | 246 | 1.27 | 0.43 | 3.74 |
| Anti_VEGF_Resistant | Remainder | rs2274700 | 10 | 265 | 1.25 | 0.45 | 3.43 |
| Anti_VEGF_Resistant | Remainder | rs1750311 | 10 | 265 | 1.21 | 0.48 | 3.09 |
| RPED | Remainder | rs698859 | 31 | 244 | 0.9 | 0.53 | 1.53 |
| Arteriolarization | Remainder | rs1409153 | 77 | 198 | 0.93 | 0.64 | 1.35 |
| Arteriolarization | Remainder | rs1061170 | 77 | 198 | 0.93 | 0.64 | 1.35 |
| Classic | Occult | rs1750311 | 89 | 171 | 0.93 | 0.62 | 1.38 |
| Classic | RAP | rs2274700 | 89 | 15 | 1.2 | 0.46 | 3.16 |
| Occult | RAP | rs1750311 | 171 | 15 | 1.17 | 0.51 | 2.7 |
| Occult | RAP | rs2230199 | 171 | 15 | 0.86 | 0.37 | 1.95 |
| Occult | RAP | H4 | 171 | 15 | 1.33 | 0.28 | 6.2 |
| Anti_VEGF_Resistant | Remainder | rs403846 | 10 | 265 | 0.85 | 0.34 | 2.11 |
| PPP | Remainder | rs1061170 | 29 | 246 | 1.1 | 0.64 | 1.9 |
| Polyps | Remainder | H1 | 115 | 160 | 1.12 | 0.59 | 2.11 |
| PPP | Remainder | rs698859 | 29 | 246 | 1.1 | 0.63 | 1.9 |
| Arteriolarization | Remainder | rs698859 | 77 | 198 | 1.06 | 0.73 | 1.54 |
| Polyps | Remainder | rs2274700 | 115 | 160 | 0.94 | 0.62 | 1.41 |
| Anti_VEGF_Sensitive | Remainder | rs12144939 | 88 | 187 | 1.1 | 0.6 | 2.02 |
| Anti_VEGF_Sensitive | Anti_VEGF_Dependent | rs2230199 | 88 | 58 | 1.09 | 0.61 | 1.95 |
| RPED | Remainder | rs641153 | 31 | 244 | 0.83 | 0.25 | 2.81 |
| RPED | Remainder | rs12144939 | 31 | 244 | 0.87 | 0.34 | 2.24 |
| Anti_VEGF_Sensitive | Anti_VEGF_Dependent | rs10490924 | 88 | 58 | 0.94 | 0.59 | 1.49 |
| PPP | Remainder | rs1409153 | 29 | 246 | 1.08 | 0.63 | 1.86 |
| Classic | Occult | rs403846 | 89 | 171 | 0.95 | 0.66 | 1.37 |
| Anti_VEGF_Resistant | Remainder | rs10490924 | 10 | 265 | 1.12 | 0.48 | 2.62 |
| Anti_VEGF_Resistant | Remainder | rs1409153 | 10 | 265 | 0.89 | 0.36 | 2.17 |
| Polyps | Remainder | rs403846 | 115 | 160 | 0.96 | 0.68 | 1.34 |
| Anti_VEGF_Dependent | Remainder | rs9332739 | 58 | 217 | 0.82 | 0.17 | 3.89 |
| Arteriolarization | Remainder | rs10490924 | 77 | 198 | 1.04 | 0.73 | 1.49 |
| Occult | RAP | rs641153 | 171 | 15 | 0.84 | 0.19 | 3.65 |
| Classic | RAP | rs641153 | 89 | 15 | 0.82 | 0.16 | 4.19 |
| Polyps | Remainder | rs10490924 | 115 | 160 | 1.04 | 0.75 | 1.44 |
| Classic | RAP | rs698859 | 89 | 15 | 0.92 | 0.43 | 1.95 |
| PPP | Remainder | H2 | 29 | 246 | 1.1 | 0.45 | 2.72 |
| Anti_VEGF_Resistant | Remainder | H1 | 10 | 265 | 0.84 | 0.17 | 4.09 |
| Anti_VEGF_Sensitive | Anti_VEGF_Dependent | rs10922153 | 88 | 58 | 0.94 | 0.56 | 1.59 |
| Polyps | Remainder | rs1409153 | 115 | 160 | 1.04 | 0.74 | 1.45 |
| Classic | Occult | H4 | 89 | 171 | 1.07 | 0.55 | 2.1 |
| Classic | RAP | rs1750311 | 89 | 15 | 1.09 | 0.45 | 2.68 |
| Anti_VEGF_Sensitive | Anti_VEGF_Dependent | H3 | 88 | 58 | 0.93 | 0.45 | 1.92 |
| Polyps | Remainder | rs1061170 | 115 | 160 | 0.97 | 0.69 | 1.36 |
| Polyps | Remainder | H3 | 115 | 160 | 1.05 | 0.63 | 1.73 |
| PPP | Remainder | rs9332739 | 29 | 246 | 0.84 | 0.1 | 6.77 |
| RPED | Remainder | H4 | 31 | 244 | 0.92 | 0.34 | 2.55 |
| Classic | RAP | rs2990510 | 89 | 15 | 0.94 | 0.39 | 2.27 |
| Classic | Occult | rs12144939 | 89 | 171 | 0.96 | 0.52 | 1.78 |
| Anti_VEGF_Resistant | Remainder | rs641153 | 10 | 265 | 0.87 | 0.11 | 6.7 |
| Anti_VEGF_Sensitive | Anti_VEGF_Dependent | H1 | 88 | 58 | 1.06 | 0.42 | 2.67 |
| Arteriolarization | Remainder | rs2274700 | 77 | 198 | 1.03 | 0.66 | 1.61 |
| Anti_VEGF_Resistant | Remainder | rs10922153 | 10 | 265 | 0.95 | 0.37 | 2.42 |
| Classic | Occult | rs10922153 | 89 | 171 | 0.98 | 0.67 | 1.43 |
| Anti_VEGF_Sensitive | Anti_VEGF_Dependent | rs1750311 | 88 | 58 | 1.03 | 0.6 | 1.76 |
| Anti_VEGF_Sensitive | Remainder | rs403846 | 88 | 187 | 1.02 | 0.71 | 1.46 |
| Classic | RAP | rs10922153 | 89 | 15 | 0.96 | 0.43 | 2.16 |
| Classic | Occult | rs1409153 | 89 | 171 | 1.01 | 0.7 | 1.45 |
| Occult | RAP | rs10922153 | 171 | 15 | 0.98 | 0.45 | 2.12 |
| Occult | RAP | H2 | 171 | 15 | 1.03 | 0.28 | 3.85 |
| Classic | Occult | rs1061170 | 89 | 171 | 0.99 | 0.69 | 1.43 |
| Occult | RAP | rs2274700 | 171 | 15 | 1.01 | 0.41 | 2.48 |
| Classic | Occult | H1 | 89 | 171 | 1.01 | 0.51 | 2 |
| Polyps | Remainder | rs698859 | 115 | 160 | 1 | 0.71 | 1.4 |
| Anti_VEGF_Sensitive | Remainder | rs641153 | 88 | 187 | 1.01 | 0.47 | 2.18 |
| Classic | Occult | rs641153 | 89 | 171 | 1.01 | 0.46 | 2.2 |
| Anti_VEGF_Sensitive | Remainder | rs698859 | 88 | 187 | 1 | 0.7 | 1.43 |
| Anti_VEGF_Sensitive | Remainder | rs1061170 | 88 | 187 | 1 | 0.7 | 1.43 |
| Anti_VEGF_Sensitive | Anti_VEGF_Dependent | rs9332739 | 88 | 58 | NA | NA | NA |
| Anti_VEGF_Sensitive | Remainder | rs9332739 | 88 | 187 | NA | NA | NA |
| Classic | RAP | rs9332739 | 89 | 15 | NA | NA | NA |
| Occult | RAP | rs9332739 | 171 | 15 | NA | NA | NA |
| RPED | Remainder | rs9332739 | 31 | 244 | NA | NA | NA |

| Group 1 | p | OR_SM | 95CI_L_SM | 95CI_U_SM | p_SM |
|---|---|---|---|---|---|
| RPED | 0.023 | 1.77 | 1.05 | 2.96 | 0.0311 |
| Polyps | 0.027 | 2.51 | 1.17 | 5.35 | 0.0176 |
| Anti_VEGF_Sensitive | 0.029 | 0.66 | 0.45 | 0.98 | 0.0404 |
| Anti_VEGF_Dependent | 0.036 | 0.61 | 0.39 | 0.96 | 0.0308 |
| Anti_VEGF_Dependent | 0.049 | 0.64 | 0.41 | 0.99 | 0.0438 |
| PPP | 0.053 | 0.56 | 0.31 | 0.99 | 0.0478 |
| Classic | 0.067 | 0.69 | 0.46 | 1.03 | 0.0699 |
| Anti_VEGF_Dependent | 0.068 | 1.47 | 0.94 | 2.3 | 0.0911 |
| Anti_VEGF_Sensitive | 0.08 | 1.59 | 0.83 | 3.07 | 0.1626 |
| PPP | 0.094 | 1.64 | 0.93 | 2.88 | 0.0882 |
| Anti_VEGF_Resistant | 0.103 | 2.27 | 0.72 | 7.1 | 0.1594 |
| RPED | 0.105 | 3.48 | 0.79 | 15.33 | 0.0989 |
| Anti_VEGF_Dependent | 0.107 | 0.65 | 0.37 | 1.12 | 0.1177 |
| Anti_VEGF_Dependent | 0.111 | 0.45 | 0.19 | 1.1 | 0.0813 |
| PPP | 0.112 | 0.46 | 0.18 | 1.2 | 0.1131 |
| Anti_VEGF_Sensitive | 0.12 | 1.52 | 0.91 | 2.52 | 0.1087 |
| Arteriolarization | 0.123 | 0.7 | 0.44 | 1.12 | 0.1381 |
| Polyps | 0.126 | 0.31 | 0.06 | 1.45 | 0.1357 |
| Anti_VEGF_Sensitive | 0.13 | 0.75 | 0.5 | 1.14 | 0.1755 |
| Anti_VEGF_Dependent | 0.131 | 0.46 | 0.18 | 1.16 | 0.0994 |
| PPP | 0.134 | 0.49 | 0.2 | 1.21 | 0.1202 |
| Anti_VEGF_Dependent | 0.145 | 1.28 | 0.86 | 1.91 | 0.2274 |
| Polyps | 0.151 | 0.59 | 0.32 | 1.08 | 0.0873 |
| Anti_VEGF_Sensitive | 0.151 | 1.97 | 0.72 | 5.43 | 0.1877 |
| Anti_VEGF_Sensitive | 0.151 | 1.97 | 0.72 | 5.43 | 0.1877 |
| Classic | 0.156 | 0.45 | 0.15 | 1.39 | 0.1648 |
| Anti_VEGF_Sensitive | 0.157 | 1.3 | 0.91 | 1.84 | 0.1455 |
| Classic | 0.157 | 0.69 | 0.44 | 1.09 | 0.1121 |
| Classic | 0.164 | 2.48 | 0.73 | 8.45 | 0.1449 |
| Anti_VEGF_Sensitive | 0.169 | 1.44 | 0.88 | 2.35 | 0.1497 |
| PPP | 0.169 | 1.5 | 0.84 | 2.65 | 0.1679 |
| Occult | 0.175 | 1.66 | 0.78 | 3.54 | 0.1899 |
| Anti_VEGF_Dependent | 0.178 | 0.72 | 0.46 | 1.14 | 0.1581 |
| Classic | 0.187 | 0.72 | 0.41 | 1.27 | 0.2597 |
| Anti_VEGF_Resistant | 0.2 | 0.53 | 0.2 | 1.41 | 0.2064 |
| Anti_VEGF_Sensitive | 0.2 | 1.36 | 0.75 | 2.49 | 0.3134 |
| Anti_VEGF_Dependent | 0.204 | 0.68 | 0.42 | 1.12 | 0.1332 |
| Arteriolarization | 0.205 | 2.17 | 0.64 | 7.36 | 0.2118 |
| RPED | 0.22 | 0.69 | 0.38 | 1.24 | 0.2134 |
| Anti_VEGF_Dependent | 0.221 | 0.77 | 0.5 | 1.18 | 0.2282 |
| PPP | 0.221 | 1.42 | 0.82 | 2.43 | 0.208 |
| Classic | 0.226 | 1.24 | 0.86 | 1.79 | 0.2488 |
| Polyps | 0.229 | 1.52 | 0.85 | 2.74 | 0.1576 |
| Classic | 0.237 | 1.34 | 0.73 | 2.46 | 0.3509 |
| Arteriolarization | 0.243 | 1.51 | 0.81 | 2.82 | 0.1928 |
| Anti_VEGF_Dependent | 0.245 | 0.78 | 0.51 | 1.2 | 0.2612 |
| RPED | 0.251 | 1.36 | 0.77 | 2.39 | 0.2912 |
| Anti_VEGF_Sensitive | 0.252 | 1.41 | 0.6 | 3.31 | 0.4242 |
| Anti_VEGF_Dependent | 0.255 | 0.5 | 0.17 | 1.48 | 0.2134 |
| Classic | 0.267 | 0.57 | 0.22 | 1.48 | 0.2472 |
| Anti_VEGF_Sensitive | 0.267 | 0.8 | 0.46 | 1.41 | 0.4429 |
| Polyps | 0.279 | 1.52 | 0.8 | 2.89 | 0.1979 |
| Anti_VEGF_Resistant | 0.28 | 1.92 | 0.47 | 7.88 | 0.3664 |
| Anti_VEGF_Sensitive | 0.285 | 1.25 | 0.81 | 1.91 | 0.3103 |
| Arteriolarization | 0.291 | 1.22 | 0.83 | 1.8 | 0.3087 |
| RPED | 0.291 | 1.45 | 0.79 | 2.65 | 0.2333 |
| Classic | 0.303 | 1.56 | 0.67 | 3.64 | 0.304 |
| PPP | 0.31 | 0.72 | 0.4 | 1.32 | 0.2938 |
| RPED | 0.321 | 0.71 | 0.35 | 1.43 | 0.3349 |
| Anti_VEGF_Sensitive | 0.327 | 0.77 | 0.46 | 1.3 | 0.336 |
| Arteriolarization | 0.332 | 0.69 | 0.34 | 1.38 | 0.29 |
| RPED | 0.339 | 0.77 | 0.45 | 1.33 | 0.3542 |
| Anti_VEGF_Resistant | 0.346 | 0.4 | 0.08 | 1.97 | 0.2615 |
| Anti_VEGF_Resistant | 0.348 | 2.65 | 0.3 | 23.45 | 0.3805 |
| Occult | 0.351 | 1.86 | 0.54 | 6.39 | 0.3268 |
| Anti_VEGF_Resistant | 0.352 | 0.44 | 0.05 | 3.61 | 0.4416 |
| PPP | 0.357 | 1.34 | 0.72 | 2.47 | 0.3565 |
| RPED | 0.365 | 0.78 | 0.45 | 1.35 | 0.3704 |
| RPED | 0.367 | 0.72 | 0.38 | 1.36 | 0.3153 |
| RPED | 0.368 | 0.64 | 0.23 | 1.78 | 0.3925 |
| Classic | 0.368 | 1.73 | 0.48 | 6.28 | 0.404 |
| Classic | 0.372 | 0.72 | 0.34 | 1.52 | 0.3872 |
| Anti_VEGF_Sensitive | 0.374 | 1.91 | 0.56 | 6.57 | 0.3027 |
| Classic | 0.375 | 0.87 | 0.61 | 1.23 | 0.4232 |
| Arteriolarization | 0.389 | 1.43 | 0.68 | 2.99 | 0.3481 |
| Anti_VEGF_Sensitive | 0.394 | 1.27 | 0.76 | 2.12 | 0.3546 |
| Arteriolarization | 0.395 | 1.17 | 0.78 | 1.76 | 0.4385 |
| Arteriolarization | 0.396 | 0.84 | 0.56 | 1.25 | 0.3831 |
| RPED | 0.401 | 0.79 | 0.46 | 1.37 | 0.4009 |
| Occult | 0.404 | 0.72 | 0.34 | 1.52 | 0.3851 |
| Anti_VEGF_Dependent | 0.414 | 0.85 | 0.51 | 1.42 | 0.5385 |
| PPP | 0.414 | 1.44 | 0.62 | 3.36 | 0.4019 |
| Occult | 0.416 | 0.72 | 0.34 | 1.52 | 0.3878 |
| Anti_VEGF_Sensitive | 0.423 | 1.22 | 0.6 | 2.46 | 0.5836 |
| Occult | 0.425 | 0.73 | 0.35 | 1.54 | 0.4113 |
| Occult | 0.426 | 0.61 | 0.2 | 1.88 | 0.389 |
| Polyps | 0.43 | 1.16 | 0.78 | 1.74 | 0.4668 |
| Polyps | 0.439 | 1.14 | 0.79 | 1.65 | 0.4797 |
| Arteriolarization | 0.442 | 0.73 | 0.35 | 1.52 | 0.3963 |
| PPP | 0.444 | 1.29 | 0.69 | 2.4 | 0.4302 |
| Classic | 0.457 | 1.17 | 0.76 | 1.8 | 0.487 |
| Anti_VEGF_Dependent | 0.464 | 0.8 | 0.38 | 1.7 | 0.5662 |
| Occult | 0.469 | 1.36 | 0.61 | 3.05 | 0.4561 |
| Classic | 0.48 | 0.78 | 0.37 | 1.62 | 0.5063 |
| Arteriolarization | 0.485 | 0.71 | 0.3 | 1.7 | 0.4467 |
| Anti_VEGF_Sensitive | 0.501 | 1.37 | 0.7 | 2.67 | 0.3604 |
| Arteriolarization | 0.512 | 0.8 | 0.45 | 1.42 | 0.45 |
| Occult | 0.528 | 0.77 | 0.36 | 1.64 | 0.4981 |
| Anti_VEGF_Sensitive | 0.529 | 0.92 | 0.64 | 1.31 | 0.636 |
| Classic | 0.533 | 0.8 | 0.37 | 1.73 | 0.5724 |
| Polyps | 0.543 | 0.92 | 0.64 | 1.31 | 0.6385 |
| Anti_VEGF_Sensitive | 0.561 | 1.11 | 0.75 | 1.64 | 0.6 |
| Anti_VEGF_Dependent | 0.57 | 0.82 | 0.43 | 1.57 | 0.5525 |
| Classic | 0.571 | 1.44 | 0.36 | 5.78 | 0.6035 |
| Anti_VEGF_Resistant | 0.572 | 0.78 | 0.25 | 2.44 | 0.6759 |
| RPED | 0.573 | 1.29 | 0.58 | 2.86 | 0.5346 |
| Arteriolarization | 0.579 | 0.89 | 0.61 | 1.3 | 0.5461 |
| Polyps | 0.58 | 1.14 | 0.79 | 1.67 | 0.4813 |
| PPP | 0.587 | 1.32 | 0.5 | 3.49 | 0.5722 |
| Anti_VEGF_Resistant | 0.591 | 0.73 | 0.29 | 1.84 | 0.5044 |
| Anti_VEGF_Sensitive | 0.593 | 0.87 | 0.56 | 1.34 | 0.5172 |
| Anti_VEGF_Sensitive | 0.604 | 1.14 | 0.71 | 1.82 | 0.5971 |
| Occult | 0.641 | 1.37 | 0.33 | 5.78 | 0.6675 |
| Anti_VEGF_Resistant | 0.65 | 1.22 | 0.47 | 3.12 | 0.6843 |
| Classic | 0.661 | 1.55 | 0.31 | 7.71 | 0.5948 |
| Classic | 0.661 | 1.55 | 0.31 | 7.71 | 0.5948 |
| Anti_VEGF_Dependent | 0.662 | 1.23 | 0.54 | 2.76 | 0.6244 |
| PPP | 0.668 | 1.28 | 0.43 | 3.8 | 0.6596 |
| Anti_VEGF_Resistant | 0.669 | 1.28 | 0.47 | 3.44 | 0.6278 |
| Anti_VEGF_Resistant | 0.684 | 1.12 | 0.43 | 2.89 | 0.8219 |
| RPED | 0.688 | 0.9 | 0.53 | 1.55 | 0.7113 |
| Arteriolarization | 0.701 | 0.92 | 0.64 | 1.34 | 0.6789 |
| Arteriolarization | 0.704 | 0.92 | 0.63 | 1.34 | 0.6568 |
| Classic | 0.705 | 0.95 | 0.64 | 1.42 | 0.811 |
| Classic | 0.711 | 1.21 | 0.46 | 3.23 | 0.6979 |
| Occult | 0.712 | 1.13 | 0.48 | 2.67 | 0.7751 |
| Occult | 0.712 | 0.87 | 0.38 | 2 | 0.7432 |
| Occult | 0.719 | 1.29 | 0.27 | 6.08 | 0.7451 |
| Anti_VEGF_Resistant | 0.722 | 0.81 | 0.32 | 2.02 | 0.6468 |
| PPP | 0.723 | 1.12 | 0.64 | 1.94 | 0.693 |
| Polyps | 0.73 | 1.04 | 0.54 | 1.98 | 0.9101 |
| PPP | 0.742 | 1.1 | 0.63 | 1.92 | 0.7312 |
| Arteriolarization | 0.745 | 1.07 | 0.74 | 1.55 | 0.7349 |
| Polyps | 0.754 | 0.93 | 0.61 | 1.4 | 0.7133 |
| Anti_VEGF_Sensitive | 0.754 | 1.19 | 0.64 | 2.2 | 0.578 |
| Anti_VEGF_Sensitive | 0.766 | 1.02 | 0.56 | 1.84 | 0.9522 |
| RPED | 0.768 | 0.81 | 0.24 | 2.74 | 0.7346 |
| RPED | 0.769 | 0.84 | 0.32 | 2.2 | 0.7251 |
| Anti_VEGF_Sensitive | 0.782 | 0.96 | 0.6 | 1.54 | 0.8667 |
| PPP | 0.783 | 1.09 | 0.63 | 1.89 | 0.7566 |
| Classic | 0.786 | 0.97 | 0.67 | 1.4 | 0.8724 |
| Anti_VEGF_Resistant | 0.793 | 1.06 | 0.45 | 2.5 | 0.901 |
| Anti_VEGF_Resistant | 0.795 | 0.86 | 0.35 | 2.13 | 0.7442 |
| Polyps | 0.797 | 0.98 | 0.7 | 1.39 | 0.9279 |
| Anti_VEGF_Dependent | 0.8 | 0.8 | 0.16 | 3.88 | 0.7803 |
| Arteriolarization | 0.81 | 1.03 | 0.72 | 1.48 | 0.8608 |
| Occult | 0.811 | 0.81 | 0.18 | 3.63 | 0.7864 |
| Classic | 0.814 | 0.83 | 0.16 | 4.29 | 0.8198 |
| Polyps | 0.816 | 1.04 | 0.75 | 1.44 | 0.82 |
| Classic | 0.822 | 0.92 | 0.43 | 1.98 | 0.8352 |
| PPP | 0.828 | 1.1 | 0.44 | 2.77 | 0.8362 |
| Anti_VEGF_Resistant | 0.829 | 0.95 | 0.19 | 4.74 | 0.9521 |
| Anti_VEGF_Sensitive | 0.831 | 0.97 | 0.57 | 1.64 | 0.8969 |
| Polyps | 0.833 | 1.07 | 0.76 | 1.5 | 0.7158 |
| Classic | 0.837 | 1.16 | 0.58 | 2.29 | 0.6731 |
| Classic | 0.848 | 1.11 | 0.45 | 2.72 | 0.8205 |
| Anti_VEGF_Sensitive | 0.848 | 1.1 | 0.51 | 2.36 | 0.811 |
| Polyps | 0.85 | 1.01 | 0.71 | 1.42 | 0.9644 |
| Polyps | 0.859 | 1.15 | 0.68 | 1.93 | 0.6032 |
| PPP | 0.867 | 0.84 | 0.1 | 6.83 | 0.8708 |
| RPED | 0.88 | 0.9 | 0.32 | 2.53 | 0.8465 |
| Classic | 0.882 | 0.93 | 0.38 | 2.28 | 0.8699 |
| Classic | 0.893 | 1.03 | 0.55 | 1.93 | 0.9309 |
| Anti_VEGF_Resistant | 0.897 | 0.77 | 0.1 | 5.87 | 0.801 |
| Anti_VEGF_Sensitive | 0.902 | 1.01 | 0.39 | 2.59 | 0.9879 |
| Arteriolarization | 0.906 | 1.03 | 0.66 | 1.62 | 0.8822 |
| Anti_VEGF_Resistant | 0.915 | 0.91 | 0.35 | 2.33 | 0.8374 |
| Classic | 0.918 | 1 | 0.68 | 1.46 | 0.999 |
| Anti_VEGF_Sensitive | 0.919 | 1.07 | 0.62 | 1.85 | 0.8137 |
| Anti_VEGF_Sensitive | 0.924 | 1.06 | 0.73 | 1.52 | 0.771 |
| Classic | 0.926 | 0.97 | 0.43 | 2.18 | 0.9378 |
| Classic | 0.963 | 1.03 | 0.71 | 1.48 | 0.8918 |
| Occult | 0.965 | 0.96 | 0.44 | 2.1 | 0.9108 |
| Occult | 0.966 | 1.06 | 0.28 | 4.01 | 0.934 |
| Classic | 0.966 | 1.02 | 0.71 | 1.48 | 0.9023 |
| Occult | 0.982 | 1.01 | 0.41 | 2.5 | 0.9821 |
| Classic | 0.983 | 0.93 | 0.47 | 1.87 | 0.8481 |
| Polyps | 0.986 | 1 | 0.71 | 1.4 | 0.993 |
| Anti_VEGF_Sensitive | 0.986 | 1.08 | 0.5 | 2.36 | 0.8456 |
| Classic | 0.989 | 1.08 | 0.49 | 2.38 | 0.8524 |
| Anti_VEGF_Sensitive | 0.994 | 1 | 0.7 | 1.43 | 0.9936 |
| Anti_VEGF_Sensitive | 1 | 1.05 | 0.73 | 1.52 | 0.7878 |
| Anti_VEGF_Sensitive | NA | NA | NA | NA | NA |
| Anti_VEGF_Sensitive | NA | NA | NA | NA | NA |
| Classic | NA | NA | NA | NA | NA |
| Occult | NA | NA | NA | NA | NA |
| RPED | NA | NA | NA | NA | NA |

OR, odds ratio;
95CI_L, 95% confidence interval lower,
95CI_U, 95% confidence interval upper;
p, p value; OR_SM, odds ratio smoking;
95CI_L_SM, 95% confidence interval lower smoking,
95CI_U_SM, 95% confidence interval upper smoking;
p_SM, p value smoking

TABLE 3B

| Group 1 | Group 2 | Marker | N1 | N2 | OR | 95CI_L | 95CI_U | p |
|---|---|---|---|---|---|---|---|---|
| Anti_VEGF_Dependent | Remainder | rs403846 | 58 | 269 | 0.58 | 0.38 | 0.88 | 0.01 |
| Anti_VEGF_Dependent | Remainder | rs1061170 | 58 | 269 | 0.6 | 0.4 | 0.92 | 0.02 |
| Anti_VEGF_Dependent | Remainder | rs10490924 | 58 | 269 | 1.49 | 1.02 | 2.17 | 0.04 |
| Anti_VEGF_Dependent | Remainder | rs2990510 | 58 | 269 | 1.56 | 1.01 | 2.41 | 0.05 |
| Anti_VEGF_Sensitive | Remainder | rs10922153 | 92 | 235 | 0.57 | 0.39 | 0.83 | 0 |
| Anti_VEGF_Sensitive | Remainder | rs10490924 | 92 | 235 | 1.51 | 1.09 | 2.1 | 0.01 |
| Anti_VEGF_Sensitive | Remainder | rs1750311 | 92 | 235 | 0.66 | 0.45 | 0.98 | 0.04 |
| Polyps | Remainder | rs641153 | 116 | 211 | 2.03 | 1.04 | 3.99 | 0.04 |
| RPED | Remainder | rs10490924 | 32 | 295 | 1.97 | 1.21 | 3.21 | 0.01 |
| RPED | Remainder | H1 | 32 | 295 | 4.24 | 0.99 | 18.21 | 0.05 |
| Anti_VEGF_Dependent | Remainder | rs2274700 | 58 | 269 | 0.6 | 0.36 | 1.02 | 0.06 |
| Anti_VEGF_Sensitive | Remainder | rs1409153 | 92 | 235 | 0.73 | 0.52 | 1.02 | 0.06 |
| Anti_VEGF_Dependent | Remainder | rs12144939 | 58 | 269 | 0.47 | 0.21 | 1.05 | 0.06 |
| Classic | Occult | rs2990510 | 89 | 172 | 0.69 | 0.46 | 1.02 | 0.07 |
| Polyps | Remainder | rs9332739 | 116 | 211 | 0.25 | 0.06 | 1.1 | 0.07 |
| Anti_VEGF_Dependent | Remainder | H4 | 58 | 269 | 0.47 | 0.2 | 1.08 | 0.08 |
| Anti_VEGF_Sensitive | Remainder | H1 | 92 | 235 | 1.81 | 0.94 | 3.51 | 0.08 |
| Polyps | Remainder | rs10490924 | 116 | 211 | 1.31 | 0.97 | 1.78 | 0.08 |
| Arteriolarization | Remainder | rs12144939 | 77 | 250 | 0.55 | 0.28 | 1.08 | 0.08 |
| PPP | Remainder | H3 | 29 | 298 | 0.45 | 0.18 | 1.15 | 0.09 |
| Anti_VEGF_Dependent | Remainder | rs10922153 | 58 | 269 | 0.69 | 0.45 | 1.07 | 0.1 |
| Arteriolarization | Remainder | H4 | 77 | 250 | 0.56 | 0.28 | 1.14 | 0.11 |
| Arteriolarization | Remainder | H1 | 77 | 250 | 1.73 | 0.86 | 3.5 | 0.13 |
| Anti_VEGF_Dependent | Remainder | rs1409153 | 58 | 269 | 0.73 | 0.49 | 1.09 | 0.13 |
| RPED | Remainder | rs10922153 | 32 | 295 | 0.65 | 0.37 | 1.14 | 0.13 |
| Anti_VEGF_Dependent | Remainder | rs1750311 | 58 | 269 | 0.7 | 0.44 | 1.12 | 0.14 |
| Arteriolarization | Remainder | rs10490924 | 77 | 250 | 1.29 | 0.92 | 1.82 | 0.14 |
| Polyps | Remainder | H2 | 116 | 211 | 0.66 | 0.37 | 1.16 | 0.15 |
| Polyps | Remainder | rs10922153 | 116 | 211 | 0.78 | 0.56 | 1.09 | 0.15 |
| Classic | RAP | H3 | 89 | 15 | 0.45 | 0.15 | 1.36 | 0.16 |
| RPED | Remainder | rs1409153 | 32 | 295 | 0.69 | 0.41 | 1.16 | 0.16 |
| Arteriolarization | Remainder | rs403846 | 77 | 250 | 0.77 | 0.53 | 1.11 | 0.16 |
| Classic | Occult | rs9332739 | 89 | 172 | 2.39 | 0.71 | 8.05 | 0.16 |
| Classic | Occult | rs2230199 | 89 | 172 | 0.73 | 0.47 | 1.14 | 0.17 |
| Polyps | Remainder | rs403846 | 116 | 211 | 0.8 | 0.58 | 1.1 | 0.17 |
| Polyps | Remainder | H1 | 116 | 211 | 1.5 | 0.84 | 2.71 | 0.17 |
| Occult | RAP | rs10490924 | 172 | 15 | 1.67 | 0.79 | 3.53 | 0.18 |
| RPED | Remainder | rs1061170 | 32 | 295 | 0.7 | 0.42 | 1.19 | 0.19 |
| Classic | Occult | H3 | 89 | 172 | 0.69 | 0.4 | 1.21 | 0.2 |
| Arteriolarization | Remainder | rs1409153 | 77 | 250 | 0.79 | 0.55 | 1.13 | 0.2 |
| Anti_VEGF_Resistant | Remainder | rs12144939 | 10 | 317 | 2.08 | 0.67 | 6.45 | 0.2 |
| RPED | Remainder | rs403846 | 32 | 295 | 0.71 | 0.42 | 1.21 | 0.2 |
| PPP | Remainder | rs10922153 | 29 | 298 | 1.43 | 0.82 | 2.47 | 0.21 |
| Classic | Occult | rs698859 | 89 | 172 | 1.26 | 0.88 | 1.82 | 0.21 |
| Polyps | Remainder | rs1061170 | 116 | 211 | 0.82 | 0.59 | 1.12 | 0.21 |
| PPP | Remainder | rs10490924 | 29 | 298 | 0.71 | 0.41 | 1.22 | 0.21 |
| Arteriolarization | Remainder | rs2230199 | 77 | 250 | 0.75 | 0.48 | 1.18 | 0.22 |
| Classic | Occult | H2 | 89 | 172 | 1.45 | 0.8 | 2.63 | 0.23 |
| Arteriolarization | Remainder | rs1061170 | 77 | 250 | 0.8 | 0.56 | 1.15 | 0.23 |
| RPED | Remainder | rs2274700 | 32 | 295 | 0.67 | 0.34 | 1.3 | 0.23 |
| RPED | Remainder | rs2990510 | 32 | 295 | 1.39 | 0.8 | 2.43 | 0.25 |
| Polyps | Remainder | rs2274700 | 116 | 211 | 0.8 | 0.55 | 1.17 | 0.25 |
| Anti_VEGF_Dependent | Remainder | rs641153 | 58 | 269 | 0.54 | 0.19 | 1.57 | 0.26 |
| Anti_VEGF_Sensitive | Remainder | H3 | 92 | 235 | 0.74 | 0.44 | 1.25 | 0.26 |
| Arteriolarization | Remainder | H2 | 77 | 250 | 1.39 | 0.78 | 2.5 | 0.27 |
| Classic | RAP | rs2230199 | 89 | 15 | 0.59 | 0.23 | 1.51 | 0.27 |
| Anti_VEGF_Resistant | Remainder | rs698859 | 10 | 317 | 0.59 | 0.23 | 1.53 | 0.28 |
| RPED | Remainder | rs1750311 | 32 | 295 | 0.72 | 0.4 | 1.32 | 0.29 |
| PPP | Remainder | rs2990510 | 29 | 298 | 0.73 | 0.4 | 1.33 | 0.3 |
| RPED | Remainder | rs2230199 | 32 | 295 | 1.36 | 0.76 | 2.45 | 0.3 |
| PPP | Remainder | rs1750311 | 29 | 298 | 1.34 | 0.77 | 2.36 | 0.3 |
| Anti_VEGF_Dependent | Remainder | rs698859 | 58 | 269 | 0.81 | 0.54 | 1.21 | 0.3 |
| Classic | RAP | rs10490924 | 89 | 15 | 1.56 | 0.67 | 3.65 | 0.3 |
| Anti_VEGF_Dependent | Remainder | H1 | 58 | 269 | 1.5 | 0.69 | 3.23 | 0.3 |
| Polyps | Remainder | rs2230199 | 116 | 211 | 1.22 | 0.83 | 1.77 | 0.31 |
| Anti_VEGF_Resistant | Remainder | H3 | 10 | 317 | 0.45 | 0.09 | 2.16 | 0.32 |
| RPED | Remainder | H2 | 32 | 295 | 0.61 | 0.23 | 1.63 | 0.32 |
| Polyps | Remainder | rs1409153 | 116 | 211 | 0.85 | 0.62 | 1.17 | 0.32 |
| PPP | Remainder | H1 | 29 | 298 | 0.65 | 0.27 | 1.54 | 0.32 |
| Anti_VEGF_Sensitive | Remainder | rs1061170 | 92 | 235 | 0.84 | 0.6 | 1.18 | 0.33 |
| Occult | RAP | H1 | 172 | 15 | 1.79 | 0.53 | 6.03 | 0.35 |
| Anti_VEGF_Sensitive | Remainder | H2 | 92 | 235 | 1.31 | 0.75 | 2.29 | 0.35 |
| Anti_VEGF_Resistant | Remainder | H2 | 10 | 317 | 0.37 | 0.05 | 2.98 | 0.35 |
| Anti_VEGF_Sensitive | Remainder | rs403846 | 92 | 235 | 0.85 | 0.61 | 1.2 | 0.36 |
| Arteriolarization | Remainder | rs698859 | 77 | 250 | 1.18 | 0.83 | 1.69 | 0.36 |
| PPP | Remainder | rs2230199 | 29 | 298 | 1.33 | 0.72 | 2.45 | 0.36 |
| Classic | RAP | H1 | 89 | 15 | 1.79 | 0.5 | 6.4 | 0.37 |
| Classic | RAP | rs403846 | 89 | 15 | 0.71 | 0.34 | 1.5 | 0.37 |
| Arteriolarization | Remainder | rs2990510 | 77 | 250 | 0.84 | 0.56 | 1.24 | 0.38 |
| Occult | RAP | rs698859 | 172 | 15 | 0.72 | 0.34 | 1.52 | 0.39 |
| Arteriolarization | Remainder | H3 | 77 | 250 | 0.79 | 0.46 | 1.37 | 0.4 |
| Classic | Occult | rs10490924 | 89 | 172 | 0.86 | 0.61 | 1.22 | 0.4 |
| Occult | RAP | H3 | 172 | 15 | 0.64 | 0.22 | 1.86 | 0.42 |
| Occult | RAP | rs403846 | 172 | 15 | 0.74 | 0.35 | 1.54 | 0.42 |
| Occult | RAP | rs1409153 | 172 | 15 | 0.74 | 0.35 | 1.56 | 0.43 |
| Anti_VEGF_Resistant | Remainder | rs1061170 | 10 | 317 | 0.69 | 0.28 | 1.72 | 0.43 |
| Anti_VEGF_Dependent | Remainder | H2 | 58 | 269 | 0.77 | 0.37 | 1.56 | 0.46 |
| Anti_VEGF_Resistant | Remainder | rs9332739 | 10 | 317 | 2.21 | 0.26 | 18.64 | 0.46 |
| Occult | RAP | rs2990510 | 172 | 15 | 1.35 | 0.6 | 3 | 0.47 |
| Anti_VEGF_Dependent | Remainder | H3 | 58 | 269 | 0.8 | 0.43 | 1.47 | 0.47 |
| Anti_VEGF_Resistant | Remainder | H4 | 10 | 317 | 1.66 | 0.42 | 6.6 | 0.47 |
| Arteriolarization | Remainder | rs9332739 | 77 | 250 | 1.49 | 0.5 | 4.43 | 0.47 |
| Polyps | Remainder | rs2990510 | 116 | 211 | 1.13 | 0.8 | 1.61 | 0.48 |
| PPP | Remainder | rs403846 | 29 | 298 | 1.21 | 0.71 | 2.05 | 0.48 |
| Classic | RAP | rs1409153 | 89 | 15 | 0.77 | 0.37 | 1.6 | 0.48 |
| Classic | Occult | rs2274700 | 89 | 172 | 1.17 | 0.76 | 1.8 | 0.48 |
| Arteriolarization | Remainder | rs641153 | 77 | 250 | 0.74 | 0.32 | 1.73 | 0.49 |
| PPP | Remainder | rs698859 | 29 | 298 | 1.2 | 0.7 | 2.05 | 0.51 |
| Anti_VEGF_Resistant | Remainder | rs10490924 | 10 | 317 | 1.32 | 0.58 | 3.03 | 0.51 |
| Anti_VEGF_Sensitive | Remainder | rs2990510 | 92 | 235 | 1.13 | 0.78 | 1.64 | 0.52 |
| Polyps | Remainder | rs698859 | 116 | 211 | 1.11 | 0.81 | 1.52 | 0.52 |
| Anti_VEGF_Resistant | Remainder | rs403846 | 10 | 317 | 0.75 | 0.3 | 1.86 | 0.53 |
| Occult | RAP | rs1061170 | 172 | 15 | 0.79 | 0.38 | 1.65 | 0.53 |
| Anti_VEGF_Sensitive | Remainder | rs12144939 | 92 | 235 | 0.84 | 0.48 | 1.47 | 0.53 |
| Classic | RAP | rs1061170 | 89 | 15 | 0.79 | 0.37 | 1.68 | 0.53 |
| Arteriolarization | Remainder | rs2274700 | 77 | 250 | 0.87 | 0.57 | 1.34 | 0.54 |
| Anti_VEGF_Sensitive | Remainder | rs698859 | 92 | 235 | 1.11 | 0.79 | 1.55 | 0.56 |
| Anti_VEGF_Dependent | Remainder | rs2230199 | 58 | 269 | 0.87 | 0.53 | 1.41 | 0.56 |
| Anti_VEGF_Dependent | Remainder | rs9332739 | 58 | 269 | 0.64 | 0.14 | 2.91 | 0.57 |
| Classic | RAP | H2 | 89 | 15 | 1.48 | 0.38 | 5.69 | 0.57 |
| Anti_VEGF_Resistant | Remainder | rs1409153 | 10 | 317 | 0.78 | 0.32 | 1.89 | 0.59 |
| Occult | RAP | rs12144939 | 172 | 15 | 1.44 | 0.34 | 6.06 | 0.61 |
| Anti_VEGF_Resistant | Remainder | rs2230199 | 10 | 317 | 0.76 | 0.24 | 2.35 | 0.63 |
| PPP | Remainder | rs2274700 | 29 | 298 | 1.16 | 0.63 | 2.12 | 0.64 |
| Anti_VEGF_Resistant | Remainder | rs2990510 | 10 | 317 | 1.24 | 0.48 | 3.24 | 0.66 |
| RPED | Remainder | rs12144939 | 32 | 295 | 0.82 | 0.34 | 1.98 | 0.66 |
| Classic | RAP | rs12144939 | 89 | 15 | 1.42 | 0.29 | 6.94 | 0.66 |
| Classic | RAP | H4 | 89 | 15 | 1.42 | 0.29 | 6.94 | 0.66 |
| PPP | Remainder | rs641153 | 29 | 298 | 1.26 | 0.43 | 3.7 | 0.67 |
| Anti_VEGF_Sensitive | Remainder | rs2230199 | 92 | 235 | 0.92 | 0.61 | 1.38 | 0.68 |
| Classic | Occult | rs1750311 | 89 | 172 | 0.92 | 0.62 | 1.37 | 0.68 |
| Occult | RAP | H4 | 172 | 15 | 1.37 | 0.29 | 6.41 | 0.69 |
| Occult | RAP | rs2230199 | 172 | 15 | 0.85 | 0.37 | 1.94 | 0.7 |
| PPP | Remainder | rs9332739 | 29 | 298 | 0.67 | 0.08 | 5.24 | 0.7 |
| Occult | RAP | rs1750311 | 172 | 15 | 1.18 | 0.51 | 2.73 | 0.7 |
| Classic | RAP | rs2274700 | 89 | 15 | 1.2 | 0.46 | 3.16 | 0.71 |
| RPED | Remainder | rs641153 | 32 | 295 | 0.8 | 0.24 | 2.69 | 0.72 |
| Anti_VEGF_Sensitive | Remainder | H4 | 92 | 235 | 0.9 | 0.49 | 1.64 | 0.73 |
| Anti_VEGF_Resistant | Remainder | rs10922153 | 10 | 317 | 0.86 | 0.34 | 2.17 | 0.75 |
| Arteriolarization | Remainder | rs10922153 | 77 | 250 | 1.06 | 0.74 | 1.54 | 0.75 |
| RPED | Remainder | H4 | 32 | 295 | 0.87 | 0.34 | 2.2 | 0.76 |
| RPED | Remainder | H3 | 32 | 295 | 1.12 | 0.53 | 2.38 | 0.77 |
| Classic | Occult | rs403846 | 89 | 172 | 0.95 | 0.66 | 1.37 | 0.77 |
| PPP | Remainder | rs12144939 | 29 | 298 | 1.12 | 0.49 | 2.57 | 0.79 |
| RPED | Remainder | rs698859 | 32 | 295 | 0.93 | 0.56 | 1.56 | 0.8 |
| Arteriolarization | Remainder | rs1750311 | 77 | 250 | 1.05 | 0.71 | 1.55 | 0.8 |
| Occult | RAP | rs641153 | 172 | 15 | 0.83 | 0.19 | 3.62 | 0.8 |
| Classic | RAP | rs641153 | 89 | 15 | 0.82 | 0.16 | 4.19 | 0.81 |
| PPP | Remainder | rs1409153 | 29 | 298 | 0.94 | 0.55 | 1.6 | 0.82 |
| Classic | RAP | rs698859 | 89 | 15 | 0.92 | 0.43 | 1.95 | 0.82 |
| Classic | Occult | rs12144939 | 89 | 172 | 0.93 | 0.5 | 1.73 | 0.83 |
| Anti_VEGF_Resistant | Remainder | rs1750311 | 10 | 317 | 1.11 | 0.44 | 2.83 | 0.83 |
| Anti_VEGF_Sensitive | Remainder | rs641153 | 92 | 235 | 1.08 | 0.52 | 2.24 | 0.83 |
| PPP | Remainder | H2 | 29 | 298 | 1.1 | 0.45 | 2.68 | 0.84 |
| Polyps | Remainder | H3 | 116 | 211 | 0.95 | 0.59 | 1.54 | 0.85 |
| Classic | RAP | rs1750311 | 89 | 15 | 1.09 | 0.45 | 2.68 | 0.85 |
| Anti_VEGF_Resistant | Remainder | rs2274700 | 10 | 317 | 1.1 | 0.4 | 3 | 0.86 |
| Polyps | Remainder | rs1750311 | 116 | 211 | 0.97 | 0.69 | 1.38 | 0.88 |
| Classic | RAP | rs2990510 | 89 | 15 | 0.94 | 0.39 | 2.27 | 0.88 |
| Anti_VEGF_Resistant | Remainder | rs641153 | 10 | 317 | 0.87 | 0.11 | 6.71 | 0.89 |
| PPP | Remainder | rs1061170 | 29 | 298 | 0.97 | 0.57 | 1.65 | 0.9 |
| Classic | Occult | rs10922153 | 89 | 172 | 0.98 | 0.67 | 1.43 | 0.9 |
| Polyps | Remainder | rs12144939 | 116 | 211 | 1.03 | 0.62 | 1.72 | 0.9 |
| Classic | Occult | H4 | 89 | 172 | 1.04 | 0.53 | 2.03 | 0.91 |
| Classic | RAP | rs10922153 | 89 | 15 | 0.96 | 0.43 | 2.16 | 0.93 |
| Classic | Occult | rs1061170 | 89 | 172 | 0.99 | 0.69 | 1.43 | 0.96 |
| Occult | RAP | rs2274700 | 172 | 15 | 1.02 | 0.42 | 2.51 | 0.97 |
| Anti_VEGF_Resistant | Remainder | H1 | 10 | 317 | 1.03 | 0.21 | 4.98 | 0.97 |
| Classic | Occult | rs1409153 | 89 | 172 | 1.01 | 0.7 | 1.45 | 0.97 |
| Occult | RAP | rs10922153 | 172 | 15 | 0.99 | 0.46 | 2.13 | 0.97 |
| Polyps | Remainder | H4 | 116 | 211 | 0.99 | 0.57 | 1.73 | 0.97 |
| Occult | RAP | H2 | 172 | 15 | 1.02 | 0.27 | 3.82 | 0.97 |
| Classic | Occult | rs641153 | 89 | 172 | 1.01 | 0.46 | 2.21 | 0.98 |
| Anti_VEGF_Sensitive | Remainder | rs2274700 | 92 | 235 | 1 | 0.68 | 1.49 | 0.98 |
| PPP | Remainder | H4 | 29 | 298 | 0.99 | 0.39 | 2.54 | 0.99 |
| Classic | Occult | H1 | 89 | 172 | 1 | 0.51 | 1.98 | 1 |
| Classic | RAP | rs9332739 | 89 | 15 | NA | NA | NA | NA |
| Occult | RAP | rs9332739 | 172 | 15 | NA | NA | NA | NA |
| RPED | Remainder | rs9332739 | 32 | 295 | NA | NA | NA | NA |
| Anti_VEGF_Sensitive | Remainder | rs9332739 | 92 | 235 | NA | NA | NA | NA |

| Group 1 | Group 2 | OR_SM | 95CI_L_SM | 95CI_U_SM | p_SM |
|---|---|---|---|---|---|
| Anti_VEGF_Dependent | Remainder | 0.59 | 0.38 | 0.91 | 0.018 |
| Anti_VEGF_Dependent | Remainder | 0.62 | 0.4 | 0.95 | 0.03 |
| Anti_VEGF_Dependent | Remainder | 1.36 | 0.92 | 2.01 | 0.128 |
| Anti_VEGF_Dependent | Remainder | 1.52 | 0.97 | 2.38 | 0.065 |
| Anti_VEGF_Sensitive | Remainder | 0.58 | 0.4 | 0.85 | 0.005 |
| Anti_VEGF_Sensitive | Remainder | 1.49 | 1.06 | 2.07 | 0.02 |
| Anti_VEGF_Sensitive | Remainder | 0.67 | 0.45 | 0.99 | 0.046 |
| Polyps | Remainder | 2.12 | 1.07 | 4.2 | 0.032 |
| RPED | Remainder | 1.85 | 1.12 | 3.06 | 0.016 |
| RPED | Remainder | 3.98 | 0.91 | 17.35 | 0.066 |
| Anti_VEGF_Dependent | Remainder | 0.64 | 0.38 | 1.09 | 0.1 |
| Anti_VEGF_Sensitive | Remainder | 0.75 | 0.54 | 1.06 | 0.109 |
| Anti_VEGF_Dependent | Remainder | 0.47 | 0.21 | 1.07 | 0.07 |
| Classic | Occult | 0.69 | 0.46 | 1.03 | 0.068 |
| Polyps | Remainder | 0.26 | 0.06 | 1.18 | 0.081 |
| Anti_VEGF_Dependent | Remainder | 0.47 | 0.2 | 1.11 | 0.087 |
| Anti_VEGF_Sensitive | Remainder | 1.62 | 0.83 | 3.16 | 0.162 |
| Polyps | Remainder | 1.27 | 0.92 | 1.73 | 0.141 |
| Arteriolarization | Remainder | 0.54 | 0.28 | 1.07 | 0.076 |
| PPP | Remainder | 0.45 | 0.17 | 1.15 | 0.096 |
| Anti_VEGF_Dependent | Remainder | 0.7 | 0.45 | 1.09 | 0.113 |
| Arteriolarization | Remainder | 0.56 | 0.27 | 1.13 | 0.105 |
| Arteriolarization | Remainder | 1.74 | 0.85 | 3.55 | 0.129 |
| Anti_VEGF_Dependent | Remainder | 0.76 | 0.5 | 1.15 | 0.191 |
| RPED | Remainder | 0.66 | 0.37 | 1.16 | 0.15 |
| Anti_VEGF_Dependent | Remainder | 0.67 | 0.41 | 1.08 | 0.1 |
| Arteriolarization | Remainder | 1.24 | 0.87 | 1.75 | 0.232 |
| Polyps | Remainder | 0.62 | 0.35 | 1.12 | 0.111 |
| Polyps | Remainder | 0.8 | 0.57 | 1.13 | 0.206 |
| Classic | RAP | 0.45 | 0.15 | 1.39 | 0.165 |
| RPED | Remainder | 0.71 | 0.42 | 1.21 | 0.212 |
| Arteriolarization | Remainder | 0.77 | 0.53 | 1.12 | 0.172 |
| Classic | Occult | 2.49 | 0.73 | 8.48 | 0.143 |
| Classic | Occult | 0.7 | 0.45 | 1.1 | 0.124 |
| Polyps | Remainder | 0.84 | 0.6 | 1.16 | 0.286 |
| Polyps | Remainder | 1.36 | 0.75 | 2.47 | 0.316 |
| Occult | RAP | 1.64 | 0.77 | 3.5 | 0.198 |
| RPED | Remainder | 0.74 | 0.43 | 1.26 | 0.267 |
| Classic | Occult | 0.72 | 0.41 | 1.28 | 0.266 |
| Arteriolarization | Remainder | 0.8 | 0.56 | 1.15 | 0.223 |
| Anti_VEGF_Resistant | Remainder | 1.95 | 0.63 | 6.09 | 0.249 |
| RPED | Remainder | 0.74 | 0.43 | 1.27 | 0.277 |
| PPP | Remainder | 1.46 | 0.84 | 2.55 | 0.184 |
| Classic | Occult | 1.26 | 0.87 | 1.81 | 0.223 |
| Polyps | Remainder | 0.86 | 0.62 | 1.2 | 0.38 |
| PPP | Remainder | 0.66 | 0.38 | 1.17 | 0.153 |
| Arteriolarization | Remainder | 0.77 | 0.49 | 1.2 | 0.248 |
| Classic | Occult | 1.36 | 0.74 | 2.49 | 0.327 |
| Arteriolarization | Remainder | 0.8 | 0.56 | 1.16 | 0.245 |
| RPED | Remainder | 0.7 | 0.36 | 1.38 | 0.308 |
| RPED | Remainder | 1.35 | 0.77 | 2.37 | 0.295 |
| Polyps | Remainder | 0.82 | 0.55 | 1.2 | 0.301 |
| Anti_VEGF_Dependent | Remainder | 0.51 | 0.17 | 1.48 | 0.215 |
| Anti_VEGF_Sensitive | Remainder | 0.82 | 0.48 | 1.4 | 0.477 |
| Arteriolarization | Remainder | 1.51 | 0.83 | 2.75 | 0.181 |
| Classic | RAP | 0.57 | 0.22 | 1.48 | 0.247 |
| Anti_VEGF_Resistant | Remainder | 0.58 | 0.22 | 1.52 | 0.265 |
| RPED | Remainder | 0.7 | 0.38 | 1.29 | 0.254 |
| PPP | Remainder | 0.71 | 0.39 | 1.3 | 0.271 |
| RPED | Remainder | 1.41 | 0.78 | 2.55 | 0.259 |
| PPP | Remainder | 1.34 | 0.76 | 2.36 | 0.312 |
| Anti_VEGF_Dependent | Remainder | 0.78 | 0.51 | 1.19 | 0.242 |
| Classic | RAP | 1.56 | 0.67 | 3.64 | 0.304 |
| Anti_VEGF_Dependent | Remainder | 1.38 | 0.62 | 3.05 | 0.426 |
| Polyps | Remainder | 1.22 | 0.83 | 1.79 | 0.309 |
| Anti_VEGF_Resistant | Remainder | 0.39 | 0.08 | 1.91 | 0.247 |
| RPED | Remainder | 0.62 | 0.22 | 1.71 | 0.354 |
| Polyps | Remainder | 0.89 | 0.64 | 1.22 | 0.469 |
| PPP | Remainder | 0.61 | 0.25 | 1.47 | 0.266 |
| Anti_VEGF_Sensitive | Remainder | 0.9 | 0.64 | 1.28 | 0.573 |
| Occult | RAP | 1.86 | 0.54 | 6.41 | 0.326 |
| Anti_VEGF_Sensitive | Remainder | 1.25 | 0.7 | 2.23 | 0.446 |
| Anti_VEGF_Resistant | Remainder | 0.45 | 0.05 | 3.68 | 0.454 |
| Anti_VEGF_Sensitive | Remainder | 0.9 | 0.64 | 1.28 | 0.563 |
| Arteriolarization | Remainder | 1.18 | 0.82 | 1.69 | 0.372 |
| PPP | Remainder | 1.35 | 0.73 | 2.49 | 0.341 |
| Classic | RAP | 1.73 | 0.48 | 6.28 | 0.404 |
| Classic | RAP | 0.72 | 0.34 | 1.52 | 0.387 |
| Arteriolarization | Remainder | 0.82 | 0.55 | 1.23 | 0.338 |
| Occult | RAP | 0.71 | 0.34 | 1.51 | 0.373 |
| Arteriolarization | Remainder | 0.77 | 0.44 | 1.34 | 0.356 |
| Classic | Occult | 0.88 | 0.62 | 1.25 | 0.461 |
| Occult | RAP | 0.61 | 0.2 | 1.87 | 0.388 |
| Occult | RAP | 0.72 | 0.34 | 1.53 | 0.395 |
| Occult | RAP | 0.73 | 0.35 | 1.55 | 0.416 |
| Anti_VEGF_Resistant | Remainder | 0.68 | 0.27 | 1.69 | 0.403 |
| Anti_VEGF_Dependent | Remainder | 0.81 | 0.39 | 1.71 | 0.585 |
| Anti_VEGF_Resistant | Remainder | 2.28 | 0.26 | 19.67 | 0.454 |
| Occult | RAP | 1.36 | 0.61 | 3.06 | 0.455 |
| Anti_VEGF_Dependent | Remainder | 0.81 | 0.43 | 1.54 | 0.522 |
| Anti_VEGF_Resistant | Remainder | 1.58 | 0.39 | 6.41 | 0.52 |
| Arteriolarization | Remainder | 1.53 | 0.51 | 4.59 | 0.447 |
| Polyps | Remainder | 1.11 | 0.78 | 1.58 | 0.557 |
| PPP | Remainder | 1.25 | 0.73 | 2.14 | 0.415 |
| Classic | RAP | 0.78 | 0.37 | 1.62 | 0.506 |
| Classic | Occult | 1.15 | 0.75 | 1.78 | 0.517 |
| Arteriolarization | Remainder | 0.71 | 0.3 | 1.66 | 0.431 |
| PPP | Remainder | 1.19 | 0.69 | 2.05 | 0.528 |
| Anti_VEGF_Resistant | Remainder | 1.18 | 0.5 | 2.78 | 0.703 |
| Anti_VEGF_Sensitive | Remainder | 1.11 | 0.76 | 1.62 | 0.59 |
| Polyps | Remainder | 1.1 | 0.8 | 1.52 | 0.566 |
| Anti_VEGF_Resistant | Remainder | 0.74 | 0.29 | 1.85 | 0.517 |
| Occult | RAP | 0.77 | 0.36 | 1.65 | 0.507 |
| Anti_VEGF_Sensitive | Remainder | 0.9 | 0.51 | 1.6 | 0.728 |
| Classic | RAP | 0.8 | 0.37 | 1.73 | 0.572 |
| Arteriolarization | Remainder | 0.9 | 0.59 | 1.39 | 0.642 |
| Anti_VEGF_Sensitive | Remainder | 1.09 | 0.78 | 1.54 | 0.607 |
| Anti_VEGF_Dependent | Remainder | 0.89 | 0.54 | 1.46 | 0.642 |
| Anti_VEGF_Dependent | Remainder | 0.69 | 0.15 | 3.23 | 0.642 |
| Classic | RAP | 1.44 | 0.36 | 5.78 | 0.604 |
| Anti_VEGF_Resistant | Remainder | 0.78 | 0.32 | 1.93 | 0.592 |
| Occult | RAP | 1.41 | 0.33 | 5.96 | 0.64 |
| Anti_VEGF_Resistant | Remainder | 0.81 | 0.26 | 2.49 | 0.716 |
| PPP | Remainder | 1.19 | 0.65 | 2.19 | 0.573 |
| Anti_VEGF_Resistant | Remainder | 1.2 | 0.47 | 3.11 | 0.704 |
| RPED | Remainder | 0.85 | 0.35 | 2.08 | 0.728 |
| Classic | RAP | 1.55 | 0.31 | 7.71 | 0.595 |
| Classic | RAP | 1.55 | 0.31 | 7.71 | 0.595 |
| PPP | Remainder | 1.24 | 0.42 | 3.66 | 0.693 |
| Anti_VEGF_Sensitive | Remainder | 0.9 | 0.59 | 1.37 | 0.633 |
| Classic | Occult | 0.94 | 0.63 | 1.41 | 0.774 |
| Occult | RAP | 1.34 | 0.29 | 6.28 | 0.712 |
| Occult | RAP | 0.86 | 0.38 | 1.98 | 0.729 |
| PPP | Remainder | 0.69 | 0.09 | 5.46 | 0.726 |
| Occult | RAP | 1.14 | 0.48 | 2.7 | 0.759 |
| Classic | RAP | 1.21 | 0.46 | 3.23 | 0.698 |
| RPED | Remainder | 0.78 | 0.23 | 2.61 | 0.681 |
| Anti_VEGF_Sensitive | Remainder | 0.98 | 0.53 | 1.82 | 0.96 |
| Anti_VEGF_Resistant | Remainder | 0.84 | 0.33 | 2.16 | 0.717 |
| Arteriolarization | Remainder | 1.07 | 0.73 | 1.55 | 0.735 |
| RPED | Remainder | 0.91 | 0.35 | 2.36 | 0.854 |
| RPED | Remainder | 1.2 | 0.55 | 2.62 | 0.656 |
| Classic | Occult | 0.97 | 0.67 | 1.4 | 0.851 |
| PPP | Remainder | 1.14 | 0.5 | 2.64 | 0.752 |
| RPED | Remainder | 0.91 | 0.54 | 1.55 | 0.739 |
| Arteriolarization | Remainder | 1.03 | 0.7 | 1.52 | 0.89 |
| Occult | RAP | 0.81 | 0.18 | 3.62 | 0.784 |
| Classic | RAP | 0.83 | 0.16 | 4.29 | 0.82 |
| PPP | Remainder | 0.96 | 0.56 | 1.65 | 0.891 |
| Classic | RAP | 0.92 | 0.43 | 1.98 | 0.835 |
| Classic | Occult | 0.99 | 0.53 | 1.85 | 0.975 |
| Anti_VEGF_Resistant | Remainder | 1.03 | 0.4 | 2.69 | 0.945 |
| Anti_VEGF_Sensitive | Remainder | 1.12 | 0.54 | 2.35 | 0.756 |
| PPP | Remainder | 1.13 | 0.45 | 2.8 | 0.799 |
| Polyps | Remainder | 1.05 | 0.64 | 1.71 | 0.857 |
| Classic | RAP | 1.11 | 0.45 | 2.72 | 0.821 |
| Anti_VEGF_Resistant | Remainder | 1.18 | 0.44 | 3.18 | 0.737 |
| Polyps | Remainder | 0.98 | 0.69 | 1.4 | 0.923 |
| Classic | RAP | 0.93 | 0.38 | 2.28 | 0.87 |
| Anti_VEGF_Resistant | Remainder | 0.77 | 0.1 | 5.85 | 0.801 |
| PPP | Remainder | 0.99 | 0.58 | 1.71 | 0.985 |
| Classic | Occult | 0.99 | 0.68 | 1.45 | 0.974 |
| Polyps | Remainder | 1.1 | 0.66 | 1.85 | 0.709 |
| Classic | Occult | 1.1 | 0.56 | 2.18 | 0.773 |
| Classic | RAP | 0.97 | 0.43 | 2.18 | 0.938 |
| Classic | Occult | 1.02 | 0.7 | 1.48 | 0.925 |
| Occult | RAP | 1.02 | 0.41 | 2.52 | 0.967 |
| Anti_VEGF_Resistant | Remainder | 1.07 | 0.22 | 5.3 | 0.93 |
| Classic | Occult | 1.02 | 0.71 | 1.47 | 0.908 |
| Occult | RAP | 0.96 | 0.44 | 2.11 | 0.923 |
| Polyps | Remainder | 1.07 | 0.61 | 1.88 | 0.821 |
| Occult | RAP | 1.05 | 0.28 | 3.96 | 0.947 |
| Classic | Occult | 1.08 | 0.49 | 2.39 | 0.849 |
| Anti_VEGF_Sensitive | Remainder | 1.03 | 0.69 | 1.53 | 0.902 |
| PPP | Remainder | 1.02 | 0.39 | 2.63 | 0.97 |
| Classic | Occult | 0.93 | 0.47 | 1.87 | 0.842 |
| Classic | RAP | NA | NA | NA | NA |
| Occult | RAP | NA | NA | NA | NA |
| RPED | Remainder | NA | NA | NA | NA |
| Anti_VEGF_Sensitive | Remainder | NA | NA | NA | NA |

OR, odds ratio;
95CI_L, 95% confidence interval lower, 95CI_U, 95% confidence interval upper;
p, p value;
OR_SM, odds ratio smoking;
95CI_L_SM, 95% confidence interval lower smoking, 95CI_U_SM, 95% confidence interval upper smoking;
p_SM, p value smoking

Nine tests led to p less than 0.05 without adjustment for smoking; a subset of seven had p less than 0.05 with adjustment for smoking. These results are detailed in Table 4 below.

TABLE 4
Tests with p < 0.05 without adjustment for smoking

| | | | | | | | | With Adjustment for | | |
|---|---|---|---|---|---|---|---|---|---|---|
| | | | | | No Adjustment | | | Smoking | | |
| | | | | | | 95% | | | 95% | |
| Group1 | Group2 | Marker | N1 | N2 | OR | CI | p | OR | CI | p |
| Anti-VEGF | Remainder | rs10922153 | 92 | 235 | 0.57 | (0.39, 0.83) | 0.0031 | 0.58 | (0.40, 0.85) | 0.0049 |
| Sensitive | | | | | | | | | | |
| RPED | Remainder | rs10490924 | 32 | 295 | 1.97 | (1.21, 3.21) | 0.0066 | 1.85 | (1.12, 3.06) | 0.0161 |
| Anti-VEGF | Remainder | rs403846 | 58 | 269 | 0.58 | (0.38, 0.88) | 0.0108 | 0.59 | (0.38, 0.91) | 0.0177 |
| Dependent | | | | | | | | | | |
| Anti-VEGF | Remainder | rs10490924 | 92 | 235 | 1.51 | (1.09, 2.10) | 0.0126 | 1.49 | (1.06, 2.07) | 0.0201 |
| Sensitive | | | | | | | | | | |
| Anti-VEGF | Remainder | rs1061170 | 58 | 269 | 0.6 | (0.40, 0.92) | 0.0177 | 0.62 | (0.40, 0.95) | 0.0297 |
| Dependent | | | | | | | | | | |
| Polyps | Remainder | rs641153 | 116 | 211 | 2.03 | (1.04, 3.99) | 0.0386 | 2.12 | (1.07, 4.20) | 0.0319 |
| Anti-VEGF | Remainder | rs1750311 | 92 | 235 | 0.66 | (0.45, 0.98) | 0.0392 | 0.67 | (0.45, 0.99) | 0.0461 |
| Sensitive | | | | | | | | | | |
| Anti-VEGF | Remainder | rs10490924 | 58 | 269 | 1.49 | (1.02, 2.17) | 0.0398 | 1.36 | (0.92, 2.01) | 0.1284 |
| Dependent | | | | | | | | | | |
| Anti-VEGF | Remainder | rs2990510 | 58 | 269 | 1.56 | (1.01, 2.41) | 0.0467 | 1.52 | (0.97, 2.38) | 0.0645 |
| Dependent | | | | | | | | | | |

In the comparison of mean risk score for eleven pairs of disease subtype groups, four pairs had p less than 0.05 according to the t-test. Results for all eleven pairs are given in Table 5 below with four pairs demonstrating particular levels of significance highlighted in bold.

TABLE 5
Mean Risk Score by group

| | | | | | | 95% CI for | | |
|---|---|---|---|---|---|---|---|---|
| Group1 | Group2 | Mean1 | N1 | Mean2 | N2 | Difference | T | p |
| Classic | Occult | 0.60 | 89 | 0.81 | 172 | (−0.56, 0.16) | −1.11 | 0.2695 |
| Classic | RAP | 0.60 | 89 | 0.49 | 15 | (−0.65, 0.88) | 0.32 | 0.7558 |
| Occult | RAP | 0.81 | 172 | 0.49 | 15 | (−0.43, 1.07) | 0.9 | 0.3822 |
| Polyps | Remainder | 0.71 | 116 | 0.37 | 211 | (−0.01, 0.68) | 1.93 | 0.0547 |
| Arteriolarization | Remainder | 0.66 | 77 | 0.44 | 250 | (−0.15, 0.59) | 1.18 | 0.2386 |
| RPED | Remainder | 1.27 | 32 | 0.41 | 295 | (0.38, 1.34) | 3.63 | 8.00E−04 |
| PPP | Remainder | 0.29 | 29 | 0.51 | 298 | (−0.82, 0.37) | −0.78 | 0.4426 |
| Anti_VEGF_Sensitive | Remainder | 0.90 | 92 | 0.34 | 235 | (0.22, 0.90) | 3.29 | 0.0012 |
| Anti_VEGF_Dependent | Remainder | 0.95 | 58 | 0.39 | 269 | (0.18, 0.93) | 2.95 | 0.0039 |
| Anti_VEGF_Resistant | Remainder | 0.59 | 10 | 0.49 | 317 | (−1.12, 1.33) | 0.19 | 0.8535 |
| Bilateral | Unilateral | 1.01 | 106 | 0.56 | 124 | (0.10, 0.79) | 2.53 | 0.0120 |

Mean risk scores also were stratified by smoking status, and the results are presented in Table 6 below.

TABLE 6
Mean risk score stratified by smoking status

| | | Group 1 | | | | | | Group 2 | | | | | |
|---|---|---|---|---|---|---|---|---|---|---|---|---|---|
| | | Never | | Past | | Current | | Never | | Past | | Current | |
| Group1 | Group2 | Mean | N | Mean | N | Mean | N | Mean | N | Mean | N | Mean | N |
| Classic | Occult | 0.53 | 51 | 0.91 | 16 | 0.55 | 22 | 0.78 | 93 | 1.00 | 24 | 0.77 | 55 |
| Classic | RAP | 0.53 | 51 | 0.91 | 16 | 0.55 | 22 | 0.45 | 9 | 0.25 | 2 | 0.68 | 4 |
| Occult | RAP | 0.78 | 93 | 1.00 | 24 | 0.77 | 55 | 0.45 | 9 | 0.25 | 2 | 0.68 | 4 |
| Polyps | Remainder | 0.71 | 61 | 1.15 | 23 | 0.39 | 32 | 0.16 | 139 | 0.57 | 21 | 0.88 | 51 |
| Arteriolar | Remainder | 0.48 | 41 | 0.73 | 11 | 0.94 | 25 | 0.29 | 159 | 0.93 | 33 | 0.58 | 58 |
| RPED | Remainder | 0.96 | 13 | 1.61 | 7 | 1.41 | 12 | 0.28 | 187 | 0.74 | 37 | 0.57 | 71 |
| PPP | Remainder | 0.26 | 15 | 0.18 | 5 | 0.39 | 9 | 0.33 | 185 | 0.97 | 39 | 0.73 | 74 |
| AV Sensitive | Remainder | 0.80 | 48 | 0.67 | 20 | 1.28 | 24 | 0.18 | 152 | 1.05 | 24 | 0.45 | 59 |
| AV Depend | Remainder | 1.17 | 23 | 0.56 | 12 | 0.93 | 23 | 0.22 | 177 | 0.99 | 32 | 0.60 | 60 |
| AV Resistant | Remainder | 0.08 | 4 | 2.19 | 1 | 0.69 | 5 | 0.33 | 196 | 0.85 | 43 | 0.69 | 78 |
| Bilateral | Unilateral | 0.94 | 47 | 1.05 | 17 | 1.06 | 42 | 0.55 | 73 | 0.65 | 23 | 0.53 | 28 |

### Conclusion

These results demonstrate a significant genetic association (pval less than 0.05) with 5 SNPs in CFH, CFHR5 and ARMS2 with response to anti-VEGF therapy administered to CNV subjects. These results also demonstrate a significant genetic association (pval less than 0.05) with mean risk scores and CNV phenotypic subtypes RPED versus remainder, and bilateral versus unilateral. For example, Table 5 shows a significant association of mean risk score and bilateral disease. Thus, individuals that have CNV in both eyes (i.e. bilateral) have a higher genetic burden (e.g., risk score) than individuals that have CNV in only one eye (i.e. unilateral). Patients with bilateral CNV often need higher medication dosing, more frequent injections, and/or combination therapy to maintain vision. Differences in calculated risk score were significant (pval less than 0.05) revealing anti-VEGF sensitive subjects with the lowest mean score (0.90) compared to anti-VEGF dependent subjects (0.950), subjects with bilateral disease (1.01) and subjects with RPED (1.27).

### Example 3

### Genotype Analysis of CNV Patients Treated for CNV with Anti VEGF Therapy (Ranibizumab)

### Identification of Associations Between Genotype and Phenotype

Genetic associations observed with ARMS2, Factor B, C2, C3 genotypes and CFH haplotypes (CFH haplotype were assigned for each subject) were evaluated to determine if specific gene variants predisposed individuals to the major subtypes of CNV (e.g., Classic, Occult, RAP).

Genetic associations observed with ARMS2, Factor B, C2, C3 genotypes and CFH haplotypes (CFH haplotype were assigned for each subject) were evaluated to determine if specific gene variants predisposed individuals to distinct minor subtypes of CNV (e.g., Polyps (presence=1), Arteriolarization, Retinal Pigment epithelial detachment (RPED), Peripapillary neovascularization (PPP)).

Mean risk scores were calculated and statistical significance was determined across categories for each of seven comparison groups to determine if risk score correlated with phenotypic subtypes associated with more aggressive disease (see Table 7 below). Mean risk score of subjects with bilateral disease was calculated to determine if differences between subjects with bilateral CNV (IO=1) had a mean risk score that was significantly higher compared to individuals without bilateral disease (IO=0), to determine if risk score correlated with disease severity. The impact of smoking was accounted for (never=0, past=1, current=2) as a covariate to the above analysis.

TABLE 7

| | Minor | | | | AntiVEGF | AntiVEGF |
|---|---|---|---|---|---|---|
| SNP | Allele | RPED | PPP | Polyps | Sensitive | Dependent |
| Rs1061170 | T | X | X | X | X | Less |
| | | | | | | Associated |
| | | | | | | than |
| | | | | | | Remainder |
| | | | | | | group |
| Rs403846 | G | X | X | X | X | Less |
| | | | | | | Associated |
| | | | | | | than |
| | | | | | | Remainder |
| | | | | | | group |
| Rs10490924 | T | 1.81 X More | Less | X | X | X |
| | | Associated | Associated | | | |
| | | with RPED | than remainder | | | |
| | | than | group | | | |
| | | remainder | | | | |
| | | group | | | | |
| | | (Risk factor | | | | |
| | | for RPED) | | | | |
| Rs2990510 | G | X | X | X | X | X |
| Rs10922153 | T | X | X | X | Less | X |
| | | | | | Associated | |
| | | | | | Than | |
| | | | | | remainder | |
| | | | | | group | |
| Rs1750311 | A | X | X | X | X | X |
| Rs641153 | T | X | X | 2.34X More | X | X |
| | | | | Associated | | |
| | | | | with Polyps | | |
| | | | | than | | |
| | | | | remainder | | |
| | | | | group | | |
| | | | | (Risk factor | | |
| | | | | for Polyps) | | |

Identification of Genetic Differences Associated with Response to Therapy (Anti VEGF-Ranibizumab)

Genetic associations observed with ARMS2, Factor B, C2, C3 genotypes and CFH haplotypes (CFH haplotype have been assigned for each subject) were evaluated to determine if certain gene variants were more associated with response to anti VEGF therapy. Comparison groups included VEGF Sensitive, VEGF dependent and VEGF resistant.

Mean risk scores were calculated and statistical significance was determined between groups to determine if individuals with higher genetic burden were more likely to become VEGF dependent or resistant. The impact of smoking (never=0, past=1, current=2) was accounted for as a covariate in the analysis.

### Example 4

### Examples of Certain Embodiments

Provided hereafter are non-limiting examples of certain embodiments of the technology.

A1. A method for predicting a therapeutic effect for treating a disorder, comprising:
(a) determining a genotype at multiple polymorphic markers for nucleic acid from a subject;
(b) predicting a therapeutic effect for treating the disorder based on a composite of the markers, which composite factors in:
   (i) the genotype at each of the markers, and
   (ii) a coefficient associated with predicting the therapeutic effect for treating the disorder for each of the markers.

A1.1 The method of embodiment A1, wherein the composite also factors an associated risk value for each marker.

A1.2 The method of embodiment A1.1, comprising multiplying the coefficient by the associated risk value, thereby generating a product for each marker.

A1.3 The method of embodiment A1.2, comprising generating a sum of the products.

A1.4 The method of embodiment A1.1, A1.2 or A1.3, wherein the associated risk value is an adjusted log-odds ratio.

A2. The method of embodiment A1.4, wherein predicting a therapeutic effect for treating a disorder comprises determining a risk score that factors in the adjusted log-odds ratio for each marker.

A2.1 The method of embodiment A2, wherein predicting a therapeutic effect for treating a disorder comprises determining a risk score that factors in an individual's genotype, adjusted log-odds ratio and residual risk value.

A3. The method of embodiment A2 or A2.1, wherein the risk score Sj is calculated according to Equation A:

Sj=intercept+Σ(i to n)βi*Xi Equation A

wherein Sj is the risk score for subject j, βi is the adjusted log-odds ratio for Xi, the additively coded genotype at marker i, and n is the total number of markers.

A3.1 The method of embodiment A3, wherein predicting a therapeutic effect for treating a disorder comprises determining a mean risk score.

A4. The method of embodiment A3, wherein predicting a therapeutic effect for treating a disorder comprises determining the probability pj according to Equation B:

pj=exp(Sj)/[1+exp(Sj)] Equation B.

A5. The method of any one of embodiments A1 to A4, wherein one or more of the markers are single nucleotide polymorphic markers.

A5.1 The method of embodiment A5, wherein one or more of the single nucleotide polymorphic markers are in one or more genes chosen from age-related maculopathy susceptibility protein 2 (ARMS2), complement factor H (CFH), complement component 2 (C2), complement component 3 (C3), coagulation factor XIII B subunit (F13B), complement factor H-related 4 (CFHR4), complement factor H-related 5 (CFHR5), and complement factor B (CFB).

A5.2 The method of embodiment A5.1, wherein one or more of the single nucleotide polymorphic markers are in one or more genes chosen from age-related maculopathy susceptibility protein 2 (ARMS2), complement factor H (CFH), and complement factor H-related 5 (CFHR5).

A5.3 The method of embodiment A5.1, wherein one or more of the single nucleotide polymorphic markers are in one or more genes chosen from age-related maculopathy susceptibility protein 2 (ARMS2) and complement factor B (CFB).

A6. The method of any one of embodiments A5 to A5.3, wherein one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924, rs2230199, rs11200638, rs1061147, rs1329422, rs2300430, rs10801553, rs1329421, rs10801554, rs7529589, rs1329424, rs572515, rs10922152, rs203674, rs393955, rs381974, rs395544, rs3800390, rs3748557, rs12755054, rs1759016, and rs4151667.

A6.1 The method of embodiment A6, wherein one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A7. The method of embodiment A6, wherein two or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A8. The method of embodiment A6, wherein three or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A9. The method of embodiment A6, wherein four or more of the single nucleotide polymorphic markers are chosen from rs11061170, rs2274700, rs403846, rs12144939, rs11409153, rs1750311, rs10922153, rs698859, rs2990510, rs9332739, rs641153, rs10490924 and rs2230199,

A10. The method of embodiment A6, wherein five or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A11. The method of embodiment A6, wherein six or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A12. The method of embodiment A6, wherein seven or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A13. The method of embodiment A6, wherein eight or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A14. The method of embodiment A6, wherein nine or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A15. The method of embodiment A6, wherein ten or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A16. The method of embodiment A6, wherein eleven or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A17. The method of embodiment A6, wherein twelve or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A18. The method of embodiment A6, wherein thirteen or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

A19. The method of embodiment A6, wherein the markers are rs1061170, rs2274700, rs403846, rs12144939, rs1409153, rs1750311, rs10922153, rs698859, rs2990510, rs9332739, rs641153, rs10490924 and rs2230199.

A19.1 The method of embodiment A6, wherein the markers comprise rs1061170, rs403846, rs1750311, rs10922153, rs10490924.

A19.2 The method of embodiment A6, wherein the markers comprise rs10490924 and rs641153.

A20. The method of any one of embodiments embodiment A1 to A19.2, wherein the disorder is late stage acute macular degeneration (AMD).

A20.1 The method of embodiment A20, wherein the late stage AMD is choroidal neovascular (CNV) disease.

A21. The method of any one of embodiments A2 to A20.1, wherein risk score or probability is adjusted by one or more non-genetic factors.

A22. The method of embodiment A21, wherein the one or more non-genetic factors comprise one or more of BMI, education status and smoking.

A23. The method of any one of embodiments A2 to A20.1, risk score or probability is not adjusted by one or more non-genetic factors.

A24. The method of any one of embodiments A1 to A23, wherein the therapeutic giving rise to the therapeutic effect comprises an anti-vascular endothelial growth factor (anti-VEGF) therapeutic.

A25. The method of embodiment A24, wherein the therapeutic comprises Ranibizumab.

B1. A method for predicting a phenotypic subtype of a disorder, comprising:
(a) determining a genotype at multiple polymorphic markers for nucleic acid from a subject;
(b) predicting a phenotypic subtype of the disorder based on a composite of the markers, which composite factors in:
   (i) the genotype at each of the markers, and
   (ii) a coefficient associated with predicting the phenotypic subtype of the disorder for each of the markers.

B1.1 The method of embodiment B1, wherein the composite also factors an associated risk value for each marker.

B1.2 The method of embodiment B1.1, comprising multiplying the coefficient by the associated risk value, thereby generating a product for each marker.

B1.3 The method of embodiment B1.2, comprising generating a sum of the products.

B1.4 The method of embodiment B1.1, B1.2 or B1.3, wherein the associated risk value is an adjusted log-odds ratio.

B2. The method of embodiment B1.4, wherein predicting a phenotypic subtype of a disorder comprises determining a risk score that factors in the adjusted log-odds ratio for each marker.

B2.1 The method of embodiment B2, wherein predicting a phenotypic subtype of a disorder comprises determining a risk score that factors in an individual's genotype, adjusted log-odds ratio and residual risk value.

B3. The method of embodiment B2 or B2.1, wherein the risk score Sj is calculated according to Equation A:

Sj=intercept+Σ(i to n)βi*Xi Equation A

wherein Sj is the risk score for subject j, βi is the adjusted log-odds ratio for Xi, the additively coded genotype at marker i, and n is the total number of markers.

B3.1 The method of embodiment B3, wherein predicting a phenotypic subtype of a disorder comprises determining a mean risk score.

B4. The method of embodiment B3, wherein predicting a phenotypic subtype of a disorder comprises determining the probability pj according to Equation B:

pj=exp(Sj)/[1+exp(Sj)] Equation B.

B5. The method of any one of embodiments B1 to B4, wherein one or more of the markers are single nucleotide polymorphic markers.

B5.1 The method of embodiment B5, wherein one or more of the single nucleotide polymorphic markers are in one or more genes chosen from age-related maculopathy susceptibility protein 2 (ARMS2), complement factor H (CFH), complement component 2 (C2), complement component 3 (C3), coagulation factor XIII B subunit (F13B), complement factor H-related 4 (CFHR4), complement factor H-related 5 (CFHR5), and complement factor 8 (CFB).

B5.2 The method of embodiment B5.1, wherein one or more of the single nucleotide polymorphic markers are in one or more genes chosen from age-related maculopathy susceptibility protein 2 (ARMS2), complement factor H (CFH), and complement factor H-related 5 (CFHR5).

B5.3 The method of embodiment B5.1, wherein one or more of the single nucleotide polymorphic markers are in one or more genes chosen from age-related maculopathy susceptibility protein 2 (ARMS2) and complement factor B (CFB).

B6. The method of any one of embodiments B5 to B5.3, wherein one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924, rs2230199, rs11200638, rs1061147, rs1329422, rs2300430, rs10801553, rs1329421, rs10801554, rs7529589, rs1329424, rs572515, rs10922152, rs203674, rs393955, rs381974, rs395544, rs3800390, rs3748557, rs12755054, rs1759016, and rs4151667.

B6.1 The method of embodiment B6, wherein one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B7. The method of embodiment B6, wherein two or more of the single nucleotide polymorphic markers are chosen from rs1061170, rs2274700, rs403846, rs12144939, rs1409153, rs1750311 rs10922153, rs698859, rs2990510, rs9332739, rs641153, rs10490924 and rs2230199.

B8. The method of embodiment B6, wherein three or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B9. The method of embodiment B6, wherein four or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B10. The method of embodiment B6, wherein five or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B11. The method of embodiment B6, wherein six or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B12. The method of embodiment B6, wherein seven or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B13. The method of embodiment B6, wherein eight or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B14. The method of embodiment B6, wherein nine or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B15. The method of embodiment B6, wherein ten or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B16. The method of embodiment B6, wherein eleven or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B17. The method of embodiment B6, wherein twelve or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B18. The method of embodiment B6, wherein thirteen or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

B19. The method of embodiment B6, wherein the markers are rs1061170, rs2274700, rs403846, rs12144939, rs1409153, rs1750311, rs10922153, rs698859, rs2990510, rs9332739, rs641153, rs10490924 and rs2230199.

B19.1 The method of embodiment B6, wherein the markers comprise rs1061170, rs403846, rs1750311, rs10922153, rs10490924.

B19.2 The method of embodiment B6, wherein the markers comprise rs10490924 and rs641153.

B20. The method of any one of embodiments embodiment B1 to B19.2, wherein the disorder is late stage acute macular degeneration (AMD).

B20.1 The method of embodiment B20, wherein the late stage AMD is choroidal neovascular (CNV) disease.

B21. The method of any one of embodiments B2 to B20.1, wherein risk score or probability is adjusted by one or more non-genetic factors.

B22. The method of embodiment B21, wherein the one or more non-genetic factors comprises one or more of BMI, education status and smoking.

B23. The method of any one of embodiments B2 to B20.1, wherein risk score or probability is not adjusted by one or more non-genetic factors.

B24. The method of any one of embodiments B20.1 to B23, wherein the phenotypic subtype is bilateral CNV.

B25. The method of any one of embodiments B20.1 to B23, wherein the phenotypic subtype is retinal pigment epithelial detachment (RPED) CNV.

C1. A method for determining risk of developing a disorder, comprising:
(a) determining the genotype at multiple polymorphic markers for nucleic acid from a subject;
(b) determining the risk of developing the disorder based on a composite of the markers, which composite factors in the genotype at each of the sites and a coefficient associated with the risk of developing the disorder for each of the sites.

C1.1. The method of embodiment C1, wherein the disorder is late stage acute macular degeneration (AMD).

C2. The method of embodiment C1 or C1.1, wherein determining the risk of disorder comprises determining a risk score that factors in the adjusted log-odds ratio for the genotype at each site.

C3. The method of embodiment C2, wherein the risk score Sj is calculated according to Equation A:

Sj=intercept+Σ(i to n)βi*Xi Equation A

wherein Sj is the risk score for subject j, βi is the adjusted log-odds ratio for Xi, the additively coded genotype at marker i, and n is the total number of markers.

C4. The method of embodiment C3, wherein determining the risk of the disorder comprises determining the probability pj according to Equation B:

pj=exp(Sj)/[1+exp(Sj)] Equation B.

C5. The method of any one of embodiments C1 to C4, wherein one or more of the markers are single nucleotide polymorphic markers.

C6. The method of embodiment C5, wherein one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C7. The method of embodiment C5, wherein two or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C8. The method of embodiment C5, wherein three or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C9. The method of embodiment C5, wherein four or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C10. The method of embodiment C5, wherein five or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C11. The method of embodiment C5, wherein six or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C12. The method of embodiment C5, wherein seven or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C13. The method of embodiment C5, wherein eight or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C14. The method of embodiment C5, wherein nine or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C15. The method of embodiment C5, wherein ten or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C16. The method of embodiment C5, wherein eleven or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C17. The method of embodiment C5, wherein twelve or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C18. The method of embodiment C5, wherein thirteen or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

C19. The method of embodiment C5, wherein the markers are rs1061170, rs2274700, rs403846, rs12144939, rs1409153, rs1750311, rs10922153, rs698859, rs2990510, rs9332739, rs641153, rs10490924 and rs2230199.

C20. The method of any one of embodiments C1.1 to C19, wherein the late stage AMD is choroidal neovascular (CNV) disease.

C21. The method of any one of embodiments C1 to C20, wherein the risk of developing the disorder, risk score or probability is adjusted by one or more environmental factors or behavior factors.

C22. The method of embodiment C21, wherein the one or more environmental factors or behavior factors comprises smoking.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, these modifications and improvements are within the scope and spirit of the technology.

The technology illustratively described herein suitably may be practiced in the absence of any element(s) not specifically disclosed herein. Thus, for example, the term “comprising” in each instance may be substituted by the term “consisting essentially of” or “consisting of.” The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. Use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). In certain instances units and formatting are expressed in HyperText Markup Language (HTML) format, which can be translated to another conventional format by those skilled in the art (e.g., “.sup.” refers to superscript formatting). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for predicting a therapeutic effect for treating late stage acute macular degeneration (AMD), comprising:
(a) determining a genotype at multiple polymorphic markers for nucleic acid from a subject; and
(b) predicting a therapeutic effect for treating the late stage AMD based on a composite of the markers, which composite factors in:
(i) the genotype at each of the markers, and
(ii) a coefficient associated with predicting the therapeutic effect for treating the late stage AMD for each of the markers.

2. The method of claim 1, wherein the composite also factors an associated risk value for each marker.

3. The method of claim 2, comprising multiplying the coefficient by the associated risk value, thereby generating a product for each marker.

4. The method of claim 3, comprising generating a sum of the products for the markers.

5. The method of claim 2, wherein the associated risk value is an adjusted log-odds ratio.

6. The method of claim 5, wherein predicting a therapeutic effect for treating late stage AMD comprises determining a risk score that factors in the adjusted log-odds ratio for each marker.

7. The method of claim 6, wherein predicting a therapeutic effect for treating late stage AMD comprises determining a risk score that factors in an individual's genotype, adjusted log-odds ratio and residual risk value.

8. The method of claim 6, wherein the risk score Sj is calculated according to Equation A:
Sj=intercept+Σ(i to n)βi*Xi Equation A
wherein Sj is the risk score for subject j, βi is the adjusted log-odds ratio for Xi, the additively coded genotype at marker i, and n is the total number of markers.

9. The method of claim 8, wherein predicting a therapeutic effect for treating late stage AMD comprises determining a mean risk score.

10. The method of claim 8, wherein predicting a therapeutic effect for treating late stage AMD comprises determining a probability pj according to Equation B:
pj=exp(Sj)/[1+exp(Sj)] Equation B.

11. The method of claim 1, wherein one or more of the markers are single nucleotide polymorphic markers.

12. The method of claim 11, wherein one or more of the single nucleotide polymorphic markers are in one or more genes chosen from age-related maculopathy susceptibility protein 2 (ARMS2), complement factor H (CFH), complement component 2 (C2), complement component 3 (C3), coagulation factor XIII B subunit (F13B), complement factor H-related 4 (CFHR4), complement factor H-related 5 (CFHR5), and complement factor B (CFB).

13. The method of claim 11, wherein one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924, rs2230199, rs11200638, rs1061147, rs1329422, rs2300430, rs10801553, rs1329421, rs10801554, rs7529589, rs1329424, rs572515, rs10922152, rs203674, rs393955, rs381974, rs395544, rs3800390, rs3748557, rs12755054, rs1759016, and rs4151667.

14. The method of claim 11, wherein one or more of the single nucleotide polymorphic markers are chosen from rs1061170, rs2274700, rs403846, rs12144939, rs1409153, rs1750311, rs10922153, rs698859, rs2990510, rs9332739, rs641153, rs10490924 and rs2230199.

15. The method of claim 11, wherein the single nucleotide polymorphic markers comprise rs1061170, rs2274700, rs403846, rs12144939, rs1409153, rs1750311, rs10922153, rs698859, rs2990510, rs9332739, rs641153, rs10490924 and rs2230199.

16. The method of claim 11, wherein the single nucleotide polymorphic markers comprise rs1061170, rs403846, rs1750311, rs10922153, rs10490924.

17. The method of claim 11, wherein the single nucleotide polymorphic markers comprise rs10490924 and rs641153.

18. The method of claim 1, wherein the late stage AMD is choroidal neovascular (CNV) disease.

19. The method of claim 6, wherein the risk score is adjusted by one or more non-genetic factors.

20. The method of claim 10, wherein the probability is adjusted by one or more non-genetic factors.

21. The method of claim 19, wherein the one or more non-genetic factors comprise one or more of BMI, education status and smoking.

22. The method of claim 20, wherein the one or more non-genetic factors comprise one or more of BMI, education status and smoking.

23. The method of claim 6, wherein the risk score is not adjusted by one or more non-genetic factors.

24. The method of claim 10, wherein the probability is not adjusted by one or more non-genetic factors.

25. The method of claim 1, wherein the therapeutic effect arises from administering a therapeutic agent.

26. The method of claim 25, wherein the therapeutic agent comprises an anti-vascular endothelial growth factor (anti-VEGF) therapeutic agent.

27. The method of claim 26, wherein the therapeutic agent comprises Ranibizumab.

28. The method of claim 1, comprising administering to the subject a therapy that causes the therapeutic effect in instances where a therapeutic effect is predicted for the subject.

29. The method of claim 28, wherein administering the therapy comprises administering a therapeutic agent to the subject.
